(19) United States
(12) Patent Application Publication
(10) Pub. No.: US 2026/0218315 A1
(43) Pub. Date: Jul. 30, 2026

(54) METHODS AND COMPOSITION FOR SINGLE CELL RNA-BASED HLA TYPING

(71) Applicant: Fred Hutchinson Cancer Center, Seattle, WA (US)

(72) Inventors: Scott N. Furlan, Edmonds, WA (US); Sami B. Kanaan, Seattle, WA (US)

(73) Assignee: Fred Hutchinson Cancer Center, Seattle, WA (US)

(21) Appl. No.: 19/452,042

(22) Filed: Jan. 16, 2026

Related U.S. Application Data
(60) Provisional application No. 63/746,160, filed on Jan. 16, 2025.

(51) Int. Cl. C12Q 1/6886; C12Q 1/6806; C12Q 1/686; C12Q 1/6869; C12Q 1/6881

(52) U.S. Cl. CPC C12Q 1/6886; C12Q 1/6806; C12Q 1/686; C12Q 1/6869; C12Q 1/6881; C12Q 2600/106; C12Q 2600/16

(57) ABSTRACT

A suite of oligonucleotides and methods that can be used for single cell RNA-based HLA typing is described. The HLA typing can inform clinical decision making based on the detection of distinct HLA types or reduced HLA expression by cells within a sample derived from a subject.

### CROSS-REFERENCE TO RELATED APPLICATION

This application claims priority U.S. Provisional Patent Application No. 63/746,160 filed Jan. 16, 2025, the entire contents of which are incorporated by reference herein.

### STATEMENT OF GOVERNMENT SUPPORT

This invention was made with government support under CA289886 awarded by the National Institutes of Health. The government has certain rights in the invention.

### REFERENCE TO SEQUENCE LISTING

The Sequence Listing associated with this application is provided in xml format in lieu of a paper copy, and is hereby incorporated by reference into the specification. The name of the text file containing the Sequence Listing is 3K54294.XML. The text is 81,920 bytes, was created on Jan. 16, 2026, and is being submitted electronically via Patent Center.

### FIELD OF THE DISCLOSURE

The present disclosure provides a suite of oligonucleotides that can be used for single cell RNA-based HLA typing. The HLA typing at the single cell level can inform clinical decision making based on the identification of cells expressing distinct HLA genotypes, thereby distinguishing donor-origin versus recipient-origin cells (where HLA mismatch is present) in the context of hematopoietic cell transplantation (HCT), as well as the identification of cells expressing altered/reduced levels of HLA within a sample derived from a subject. Other additional uses are described herein.

### BACKGROUND OF THE DISCLOSURE

Hematopoietic stem cells (HSC) are pluripotent stem cells that ultimately give rise to all types of terminally differentiated blood cells. HSC can self-renew, or can differentiate into more committed progenitor cells, and are irreversibly determined to be ancestors of only a few types of blood cell. For instance, HSC can differentiate into (i) myeloid progenitor cells which ultimately give rise to monocytes and macrophages, neutrophils, basophils, eosinophils, erythrocytes, megakaryocytes/platelets, dendritic cells, or (ii) lymphoid progenitor cells which ultimately give rise to T-cells, B-cells, and natural killer cells (NK-cells).

Hematopoietic cell transplant (HCT) is a life-saving therapy for many patients, for example those with blood cancers. HCT requires that the patient's existing hematopoietic system be removed before a transplant is received. Removal of a patient's existing hematopoietic system before a transplant is referred to as conditioning.

The human leukocyte antigen (HLA) system is a gene complex encoding the major histocompatibility complex (MHC) proteins in humans. These proteins are central to immunological responses including self versus non-self immune recognition. HLA genes are extremely polymorphic, with over 42,000 different alleles identified in the human population (as of 2025), providing the immune system with appropriate readiness to tackle the widely diverse “non-self” in nature. At each HLA locus there may be thousands of possible alleles, for example as described in Robinson et al., The iPD and IMGT/HLA database: allele variant databases; Nucleic Acids Research, 43, (2015); and Horton et al, Gene Map of the Extended Human MHC, Nature Reviews Genetics, Vol. 5, 889, (2004). In many cases, the HLA profile between a patient receiving an allogeneic HCT (allo-HCT) and cells from the donor(s) (also referred to as a graft) will be different. Therefore, if available techniques permit, physicians could assess the HLA profile of hematopoietic cells from a blood sample and/or a bone marrow aspirate from a leukemia patient post-allo-HCT to determine if they were patient-derived or donor-derived. Such information could inform the degree of donor chimerism, that is the percent of cells in a given patient that are donor (the remaining cells are of recipient origin). This is a useful parameter to follow clinically after allo-HCT, as the reemergence of recipient cells could include leukemic cells, thus a greater probability of relapse (the leading cause of mortality following treatment).

Further, one mechanism that cancer cells use to avoid immune system detection is the down-regulation or loss of expression of HLA entirely. With lowered or absent expression of HLA on malignant cells, the immune system will not detect and destroy patient-derived cancer, allowing the cancer to continue to progress. Thus, the ability to detect changes in HLA expression would provide another significant piece of clinical information for risk stratifying patients. Additionally, it is now widely recognized that the treatment of relapsed leukemia after transplant is most successful when the overall burden of disease is low. Therefore, early detection of relapsed disease is of critical importance.

### SUMMARY OF THE DISCLOSURE

The current disclosure provides systems and methods to identify HLA alleles and expression levels in subjects, for example those undergoing allo-HCT to treat leukemia. The disclosed systems and methods can utilize barcoded whole transcriptome amplicons in the context of single-cell RNA sequencing. Determining HLA alleles and expression levels on leukemic cells can guide selection of a donor at the time of initial diagnosis and can also be used to guide therapeutic decision making in the event of a relapse. For example, at initial diagnosis, a donor can be selected to have a mismatch within an HLA locus where the donor's leukemic cells express the allele at a high level with low variability. This mismatch selection would take priority over alleles that have variable expression or low expression. At a time of relapse, a patient's leukemic cells can be assessed for normal HLA expression, loss of the expression of one or more HLA allele(s), or reduced expression of HLA allele(s). If the HLA expression by the patient's leukemic cells is at an expected level for both alleles, a clinical decision could be to reinfuse donor lymphocytes from the same donor. If the HLA expression by the patient's leukemic cells is lost for one allele, a clinical decision could be to reinfuse donor lymphocytes from the same donor if the lost allele is not one targeted by the donor's lymphocytes. If, however, the lost allele is one targeted by donor's lymphocytes, these lymphocytes would be less effective following loss of the allele, and a different donor could be identified. If the HLA expression by the patient's leukemic cells is reduced, a therapy to increase HLA expression could be administered.

The current disclosure provides these advancements in clinical decision making by providing methods and tools for in-depth sequencing of targeted HLA genes on human chromosome 6 from a single-cell barcoded transcriptome. The methods rely on generating a single-cell, whole transcriptome amplified pool (the cDNA pool) from a sample of cells, in which each cDNA molecule includes a cell-barcode (CB) sequence (to link it to a specific cell) and a unique molecular identifier (UMI) sequence (to link transcript counts per each cell and eliminate PCR duplication events). Hybridization capture and enrichment of the transcripts of interest is then performed (in this case HLA transcripts). This step involves the use of custom-designed biotinylated DNA oligos to specifically hybridize cDNA molecules. Custom-designed DNA oligos with modified 3-end nucleotides to block unwanted amplification artifacts can also be used, along with one or more of PCR-enrichment of captured library with single-cell platform-specific primers, then indexing the library according to the appropriate downstream sequencing application (e.g., long read sequencing with Pacific Biosciences of California, Inc. or Oxford Nanopore Technology Limited). Computational tools can also be used to identify HLA-type based on publicly-available HLA allelic variants using the sequencing from individual cells. As indicated, this approach enables probing of the potential origin(s) and cell types in which HLA variants occur and can guide clinicians on appropriate therapeutic approaches for individual patients.

### BRIEF DESCRIPTION OF THE FIGURES

The patent or application file contains at least one drawing executed in color. Copies of this patent or patent application publication with color drawing(s) will be provided by the Office upon request and payment of the necessary fee. Applicant considers the color versions of the drawings as part of the original submission and reserve the right to present color images of the drawings in later proceedings.

FIGS. 1A, 1B. Exemplary use cases of the disclosure. (FIG. 1A) Aid in mismatch donor selection. Patients with new diagnosis of leukemia exhibit heterogeneity in expression of HLA molecules on leukemic blasts. In this hypothetical example with schematized data, a patient with a new diagnosis of leukemia is profiled using scrHLA-typing. Expression levels of each allele of HLA molecules are measured on the patient's leukemic blasts. Quantification reveals variability in expression of HLA-B as indicated by arrow. Other molecules exhibit homogenous expression at expected levels. When deciding between donors 1 or 2 each mismatched at 1 allele, scrHLAtyping can be used to prioritize donor 2 who is mismatched for A*03:01 and matched for B*35:02. This strategy prioritizes mismatch for targeting alleles with higher and more stable expression over alleles with lower expression or more variable expression. This strategy would also put less selective pressure on the allele that exhibits pre-existing heterogeneity in a patient's leukemic cells. (FIG. 1B) Risk stratification of patients at early timepoints after HCT to next therapy. Patients who are identified with a possible recurrence of leukemia after HCT can have scrHLA typing performed on a bone marrow aspirate. scrHLA typing can identify leukemic disease at very low levels of disease. scrHLA typing will provide the treating physician with the following outcomes: 1) maternal and paternal HLA molecules are detected as expressed at expected levels, 2) Loss of one or more alleles of HLA molecules are seen, 3) maternal and paternal HLA molecules are detected but at lower-than-expected levels. Each of these outcomes can dictate different forms of therapy. In outcome 1, provided patient does not have severe graft-versus-host disease (GVHD), it would be reasonable to trial donor lymphocyte infusion (DLI). In outcome 2, if the absent HLA molecule is not contained in the GVL vector (the GVL vector is defined as the list of recipient HLA molecules not present in donor cells), then it would be reasonable to trial DLI. However, if the absent molecule is contained in the GVL vector, administration of DLI would be futile and only likely to lead to GVHD, and second transplant with a new donor could be considered. Finally, should HLA molecules exhibit low levels of expression on leukemic blasts, it would be reasonable to trial hypomethylation therapy, which has been shown to increase expression of HLA, alone or in combination with DLI. Triaging alloHCT patients with scrHLA-typing. scrHLA-typing can show the following results on leukemic cells: 1) maternal and paternal HLA molecules (Class I and II) are detected at typical levels, 2) Loss of one or more alleles of HLA are seen, 3) maternal and paternal HLA molecules are detected but at low levels. Possible clinical outcomes are suggested.

FIGS. 2A-2E. Schematic of embodiments disclosed herein. (FIG. 2A) Hybridization pull-down. Adding blocking oligos and biotinylated hybridization capture probes to a pool of barcoded cDNA generated through a single-cell capture platform. The capture is carried forward at 65° C., and captured sequences are bound to magnetic streptavidin beads and pulled down on magnets. (FIG. 2B) Washing away unwanted/untargeted transcripts and subsequent PCR enrichment. The targeted transcripts are then amplified using universal PCR forward and reverse oligos, provided by the single-cell capture platform. (FIG. 2C) Ligation of platform-specific oligonucleotide Adaptors and sequencing. Depending on the sequencing platform to be used for long-read sequencing, the appropriate adaptor oligos are ligated to the enriched cDNA pool and loaded into the appropriate sequencer. (FIG. 2D) Sequencing data are then computationally processed using pipelines available from the sequencing platform of choice (i.e., pipelines designed to read and hybridize to the platform-specific oligonucleotide adaptors that were added, recognize the cell barcodes (CB) and unique molecular identifiers (UMI), error correct sequences, and remove duplicate sequences). The resulting sequences are then aligned to a reference genome including all the 42,000 known HLA sequences. The reads at each reference HLA are then counted and summarized. The CB-UMI of these mapped reads are then matched to previously generated single cell transcriptome data (using a different method) for the same patient and integrated for visualization and interpretation on the Uniform Manifold Approximation and Projection (UMAP) embedding (generated using R). (FIG. 2E) Summary schematic of molecular biology workflow of the disclosed scrHLA-typing methods.

FIGS. 3A-3E. scrHLA typing recapitulates clinical HLA typing in a patient with relapsed leukemia after HCT in the expected cell types at the single cell level. (FIG. 3A, FIG. 3B) UMAP embedding of single cell RNA sequencing profiles from a patient with relapsed acute myeloid leukemia after cell type annotation (FIG. 3A) and genetic demultiplexing (FIG. 3B). (FIG. 3C) Table showing clinical HLA typing. (FIG. 3D, FIG. 3E) UMAP plots showing cells with expression of HLA alleles shared between donor and recipient (FIG. 3D) and unique to donor (top) and recipient (bottom). Abbreviations: Prog—Progenitors; HSC—Hematopoietic Stem Cell; pDC, cDC—plasmacytoid and classical dendritic cells, respectively; Mono—Monocytes; NK—Natural Killer; MHC—Major Histocompatibility Complex; HLA—Human Leukocyte Antigen. HCT—hema-topoietic cell transplantion.

FIG. 4. Depiction of exemplary workflows of the disclosure.

FIGS. 5A-5E. Method schematization and comparison across single-cell capture chemistries. (FIG. 5A) Single-cell barcoded cDNA pools are combined with custom-designed biotinylated hybridization probes and blocking oligos. Enriched HLA transcripts are depleted of template switch oligo (TSO) priming artifacts, PCR-amplified, and prepared for long-read sequencing. (FIG. 5B) PacBio HiFi reads are processed with the IsoSeq pipeline to generate BAM files, serving as the input for the first ‘unrestricted’ scrHLAtag iteration (green line). scrHLAmatrix predicts a list of top HLA candidates, which is then provided back to scrHLAtag for one or more ‘restricted’ iterations (red line) until a final candidate list is obtained. Count files from the final round are summarized into a sparse numeric matrix. (FIG. 5C) Left: Top 10 most numerous alleles in the example of HLA-A generated in the first unrestricted scrHLAtag iteration for a sample from patient ‘AML1’; right: Top 10 most numerous genotype configurations in the example of HLA-A generated in the final unrestricted scrHLAtag iteration for the same sample from patient ‘AML1’. (FIG. 5D) UMAP embedding of profiles colored by cell type and genetic demultiplexing result (inset) from patient ‘AML1’ captured using 3-prime and 5-prime chemistries. (FIG. 5E) Genotyping result of donor and recipient cells from patient ‘AML1’ using scrHLA-typing. The fraction of cells expressing each allele, stratified by donor vs. recipient classification, illustrated for two genes without donor-recipient mismatches (HLA-A, HLA-B) and two genes with mismatches (HLA-DMA, HLA-DMB).

FIG. 6. Experimental design and output of targeted gene mutation capture in AML1. Using ‘mutCaller’ (a current pipeline designed for identifying mutations in single cell data) to identify two known mutations in the IDH1 and TP53 genes (clinically assessed, in patient file) and a third arbitrary mutation on the PTPN11 gene not known to be present within the patient (negative control), mutated IDH1 and TP53 transcripts were found associated with cell barcodes later identified as leukemia cells. By counting mutations on UMIs per each cell barcode (CB), cells were either expressing mutated transcripts, a combination of mutated and wildtype transcripts (biallelic), wildtype transcripts, or expression of targeted transcript was not detected (undetermined). Classification of cells by genetic demultiplexing (souporcell) into recipient and donor is also shown for reference.

FIGS. 7A-7C. Quantifying HLA alleles longitudinally after post-transplant relapse. (FIG. 7A) UMAP embedding colored by genetic demultiplexing result from patient AML4 BMMCs at days 90, 140, and 163 post-allo-HCT. (FIG. 7B) PCR duplication rates in raw count data from scrHLAtag for 2 time points: ‘AML4 day 90’ and ‘AML4 day 163’, using either the standard IsoSeq PacBio method or the augmented MAS-seq method. (FIG. 7C) Fraction of cells expressing each of the three HLA-A alleles, stratified by donor vs. recipient classification (souporcell), for each of the three ‘AML4’ time points. Normalized expression values for each allele are shown alongside as a quantitative measure.

FIG. 8. Identifying best quality reads according to minimap2 tags. Score thresholds of good quality mappings according to the ‘s1’ (chaining score), ‘AS’ (dynamic programming alignment score), ‘NM’ (total number of mismatches and gaps in the alignment), and ‘de’ (gap-compressed per-base sequence divergence) tags, where the sum of True Positive Rates (donor or recipient mappings correctly validated by souporcell) and True Negative Rates (donor or recipient mismappings as predicted by scores, correctly refuted by souporcell) was maximized at each HLA locus, for each of the tags (purple dotted line represents the mean threshold of each gene).

FIGS. 9A-9C. Correlation of HLA allele-specific transcript counts with cell surface HLA molecular expression. (FIG. 9A) UMAP embedding of bone marrow mononuclear cell profiles of ‘AML4’ colored by cell type. (FIG. 9B) The effect size (spearman rho) and statistical significance (p-values, spearman) across cell types for the relationship between per-cell counts of donor-specific (mismatched relative to the recipient) and shared HLA alleles in donor cells, and vice versa, recipient-specific and shared HLA alleles in recipient cells, with the corresponding cell-surface HLA molecules. (FIG. 9C) Example correlations between transcript counts for DRA*01:01:01 and DRA*01:03 and cell-surface DRA molecule counts in the late erythroid subset from both recipient and donor cells of ‘AML4’ at day 90 post-alloHCT.

FIGS. 10A-10B. Putative molecular swap mechanism in the case of cell barcodes at the 5′ end of the HLA transcript. (FIG. 10A) Schematic of the molecular swap process. (FIG. 10B) Molecular swap prevalence depending on whether the swap is between alleles of the same gene or different genes but of the same HLA class (i.e., intraclass swap), or between alleles distantly homologous, e.g., of a different HLA class (i.e., interclass swap).

FIGS. 11A-11E. Differential Allele-Specific Expression (ASE) across the HLA haplotype in different groups of cells. (FIG. 11A) HLA gene positions on chromosome 6 with coordinate ‘0’ corresponding to position 29,722,775 bp of the hg38 primary assembly (position of pseudogene HLA-Y was recently described in a 60,000 bp indel polymorphism [ref. Alexandrov, N., et al., HLA 102, 599-606 (2023)] between HLA-A and HLA-E). (FIG. 11B) Mean normalized expression in the example of HLA-DRB1, for the recipient-specific (DRB1*04:01:01) and the shared (DRB1*03:02:01) alleles across the cell categories and timepoints. (FIG. 11C) Example using HLA-DRB1, showing mean normalized expression of the recipient-specific allele (DRB1*04:01:01) and the shared allele (DRB1*03:02:01) across cell categories and time points. (FIG. 11D) Fold change in the proportion of cells expressing the mismatched allele (under selective pressure) vs. the shared allele, along with statistical significance (Chi-square test), shown across cell categories and time points. (FIG. 11E) Proportion of HLA genes exhibiting ‘balanced’ vs. ‘differential’ allele expression among genes with recipient-donor mismatches. ‘Differential’ expression is defined here as a fold change in the proportion expressed of mismatched vs. shared alleles>2 (either increase or decrease) and a Chi-square p-value<0.01.

FIGS. 12A-12B. HLA pattern-based classification of cells by scrHLAmatrix from in-silico mixes with 3 or more chimeric entities. The plots show classification of cells based on HLA expression patterns (‘NA’ corresponds to ‘not assigned’) in comparison to their original labeling, known a priori before BAM file merging. The examples shown here are from first guided iteration results. The prediction accuracy, defined as the proportion of cells with a given classification corresponding to the same majority label, is also shown.

FIGS. 13A-13B. Mismatch patterns in HLA sequencing reads across single cells. (FIG. 13A) Per-base mismatch rates in aligned reads, calculated relative to the assigned IMGT/HLA reference coding sequence (CDS), for HLA-A in ‘AML4’ at day 90 post-allo-HCT. Cells are classified by barcode (CB) as healthy donor, healthy recipient, or malignant recipient. Mismatch profiles are shown alongside sequencing coverage tracks (purple). For clarity, reads from erroneous allele calls (e.g., donor-specific alleles in recipient cells or vice versa, recipient-specific alleles in donor cells)—which typically display higher variant rates and markedly lower apparent coverage—are omitted. (FIG. 13B) Per-base mismatch rates and coverage for HLA-A in ‘AML8’, highlighting a 3-end insertion in A*11:01:01 affecting half the reads (brown arrow), observed in healthy donor, healthy recipient, and malignant recipient cells.

FIG. 14. Putative molecular swap mechanism in the case of cell barcodes at the 3′ end of the HLA transcript. Schematic of the molecular swap process.

FIG. 15. Quantifying the mismatched HLA alleles in samples from the same patient captured in 2 different 10× Genomics chemistries. Normalized expression of DMA*01:01:01, DMA*01:02:01, DMB*01:01:01, and DMB*01:03:01 (HLA-DMA and -DMB genes were the only genes with allele mismatches between donor and recipient).

FIG. 16. Read count metrics from alignment reads across the various processing steps, comparing 3′ vs. 5′ 10× Genomics capture chemistries. Number of reads retained at each processing step in the making of sparse numeric matrices from raw read counts.

FIG. 17. HLA pattern-based classification of cells by scrHLAmatrix. Classification of cells based on HLA expression patterns.

FIGS. 18A-18C. Read count metrics from alignment reads across the various processing steps comparing the standard Iso-Seq method to the augmented MAS-seq method. (FIG. 18A) Processing steps in the making of the sparse numeric matrices from raw read counts. (FIG. 18B) Number of reads retained at each processing step. (FIG. 18C) Number of cell-dataset-object associated cell barcodes (CBs) matching with the CBs in the read counts at each processing step.

FIG. 19. Rates of false positives or out-of-place occurrences (i.e., in reference to souporcell classification, donor-specific alleles in recipient cells and vice-versa, recipient-specific alleles in donor cells) according to the proportion of the analyzed allogeneic entity in the chimeric mix. With a maximum<5%, the rate rapidly decreases in the chimeric population as its chimerism proportion increases, maintaining a mean rate of 0.25% (purple line).

FIGS. 20A-20C. Identifying malignant cells in the AML4 samples across the 3 timepoints post-transplant. (FIG. 20A) Cell type classification according to viewmastR, split by genetic demultiplexing classification (souporcell). (FIG. 20B) Cell type composition, split by genetic demultiplexing classification (souporcell). (FIG. 20C) Identifying candidate cells (clustered together in UMAP space; almost entirely non-overlapping with ‘healthy’ clusters; indicated inside dotted circles in FIG. 20A) among recipient cells as malignant and verifying their expression of genes implicated in cellular immortality.

FIGS. 21A-21B. Experimental design and output of targeted gene fusion capture in AML4 and AML6. (FIG. 21A) Starting product is single-cell barcoded cDNA pools (from a capture platform like 10× Genomics). To that, custom-designed biotinylated hybridization probes (specific to the fusion breakpoint region) and blocking oligos are added. Transcripts with the targeted gene fusions are enriched magnetically (using streptavidin beads), depleted from template switch oligo (TSO) priming artifacts, PCR-amplified, and prepped for long-read sequencing. (FIG. 21B) Expression levels (counts) of the target-captured gene fusion transcripts by single cell.

FIGS. 22A-22C. Using additional samples to quantifying allele-specific differential expression across the HLA haplotype in groups of cells. (FIG. 22A) Genotyping of patients and their donors according to scrHLAtyping. (FIG. 22B) Mean normalized expression and proportion of cells expressed for the recipient-specific, shared (or homozygous), and donor-specific HLA alleles in the healthy donor, healthy recipient, and malignant recipient cells, for each sample. (FIG. 22C) Proportion expressed fold change of the mismatched allele (under pressure) vs. shared allele and its statistical significance (Chi-square test), for each sample.

FIG. 23. The ability of the scrHLAmatrix classifier, using the consensus algorithm, in correctly predicting the chimeric entities (ground truth: genetic demultiplexing by souporcell) as a function of the abundance of mismatched alleles between the chimeric entities.

FIG. 24. mRNA (cDNA) and genomic reference library. For each sample, the table shows the number of alleles used by scrHLAtag in its curated reference vs. the number of HLA alleles identified at the end of a run. As the first scrHLAtag run (i.e., first iteration) is “unguided”, all the alleles at the disposal of scrHLAtag are used in the reference (35141 in the mRNA reference library and 16916 in the genomic reference library). After each iteration, the number of identified alleles are reduced to the most likely alleles using the function scrHLAmatrix::Top_HLA_listo with arguments at default values. The list of HLA alleles converges to the “putative” ones after 5 to 9 iterations.

FIG. 25. Patient characteristics and clinically available HLA typing.

### DETAILED DESCRIPTION

Hematopoietic stem cells (HSC) are pluripotent stem cells that ultimately give rise to all types of terminally differentiated blood cells. HSC can self-renew, or can differentiate into more committed progenitor cells are irreversibly determined to be ancestors of only a few types of blood cell. For instance, HSC can differentiate into (i) myeloid progenitor cells which ultimately give rise to monocytes and macrophages, neutrophils, basophils, eosinophils, erythrocytes, megakaryocytes/platelets, dendritic cells, or (ii) lymphoid progenitor cells which ultimately give rise to T-cells, B-cells, and natural killer cells (NK-cells).

Hematopoietic cell transplant (HCT) is a life-saving therapy for many patients, for example those with blood cancers. Hematopoietic cell transplantation requires that the patient's existing hematopoietic system be removed before a transplant is received. Removal of a patient's existing hematopoietic system before a transplant is referred to as conditioning.

The human leukocyte antigen (HLA) system is a gene complex encoding the major histocompatibility complex (MHC) proteins in humans. These proteins are central to immunological responses including self-vs.-non-self immune recognition. HLA genes are highly polymorphic, with over 42,000 different alleles identified in the human population (as of 2025: see (hla.alleles.org); Robinson et al., The IPD and IMGT/HLA database: allele variant databases; Nucleic Acids Research, 43, (2015); and Horton et al., Gene Map of the Extended Human MHC, Nature Reviews Genetics, Vol. 5, 889, (2004)) providing the immune system with appropriate readiness to tackle the widely diverse “non-self” in nature. The HLA gene complex resides on a 3 Mbp stretch within chromosomal segment 6p21. The HLA locus comprises genes coding MHC class I molecules (including the classical HLA-A, HLA-B, HLA-C, and the non-classical HLA-E, HLA-F, HLA-G, MICA, MICB, HFE and 17 pseudogenes) and genes coding MHC class II molecules (including the classical HLA-DR, -DQ, -DP and pseudogenes, and the non-classical HLA-DM and -DO). “HLA allele” refers to any allele at the HLA locus. As used herein, “all known HLA alleles” refers to those described in Robinson et al., The IPD and IMGT/HLA database: allele variant databases; Nucleic Acids Research, 43, (2015); and Horton et al., Gene Map of the Extended Human MHC, Nature Reviews Genetics, Vol. 5, 889, (2004).

The HLA profile between a patient receiving an allo-HCT and the donor(s) of the stem cells (aka graft) is often different. Therefore, if available techniques permit, physicians could assess the HLA profile of hematopoietic cells within a patient to determine if they are patient derived or donor derived. Such information could allow detection of the reemergence of recipient cells, leukemia cells, or a combination of both, post-transplant. This assessment is significant as it informs on the risk of relapse, a leading cause of mortality, following treatment, giving a chance for patients with the highest risk of disease reoccurrence a chance for early intervention.

Further, one mechanism that cancer cells use to avoid immune system detection is the down-regulation or loss of expression of HLA entirely. With lowered or absent expression of HLA on malignant cells, the immune system will not detect and destroy patient-derived cancerous cells, allowing the cancer to continue to progress. Thus, the ability to detect changes in HLA expression would provide another significant piece of clinical information for risk stratifying patients. Additionally, it is now widely recognized that the treatment of relapsed leukemia after transplant is most successful when the overall burden of disease is low. Therefore, early detection of relapsed disease is of critical importance.

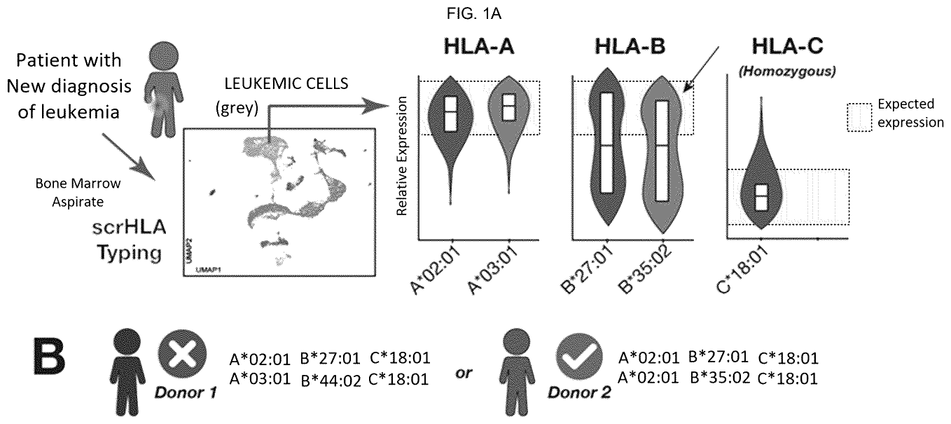

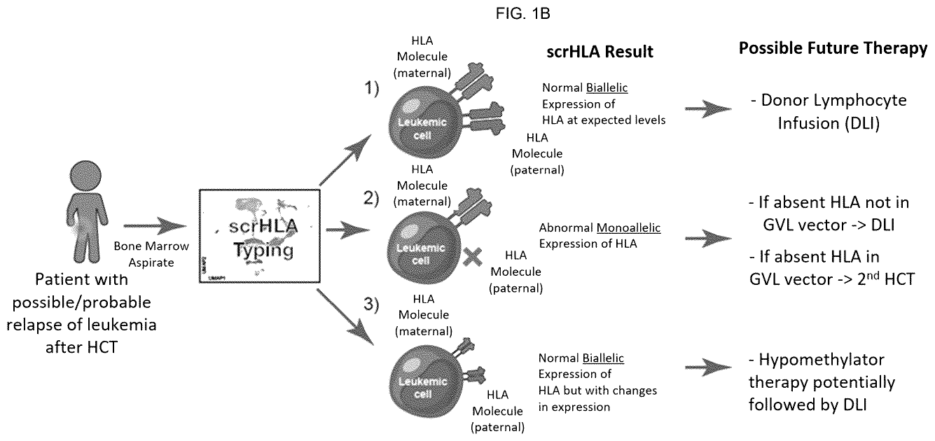

The current disclosure provides systems and methods to identify HLA alleles and/or expression levels. The systems and methods can be used in guiding the treatment of subjects undergoing allo-HCT to treat leukemia. The systems and methods utilize barcoded whole transcriptome amplicons based on single-cell RNA sequencing. Determining HLA alleles and expression levels on cancer cells can guide selection of a donor at the time of initial diagnosis and can also be used to guide therapeutic decision making in the event of a relapse. For example, at initial diagnosis, a donor can be selected to have a mismatch within an HLA locus where the donor's cancer cells express the allele at a high level with low variability. This mismatch selection would take priority over alleles that have more variable expression or low expression. This strategy is depicted in FIG. 1A, where a patient's leukemic cells are HLA typed shortly after diagnosis. Within the patient's HLA-A allele, the patient expresses alleles A*02:01 and A*03.01 at a high level with low variability, as compared to expression of HLA-B and HLA-C alleles. Within the patient's HLA-B allele, the patient expresses alleles B*27:01 and B*35.02 at a variable level (i.e., cells with high expression levels for these alleles and cells low expression levels for these alleles). This comparison is in relation to the patient's HLA-A and HLA-C expression levels. Within the HLA-C locus, the patient is homozygous, expressing only one allele at a low level, as compared to the expression levels of HLA-A and HLA-B. In this scenario, a donor with a mismatch in HLA-C would not be ideal because expression levels to target HLA-C alleles are low. A donor with a mismatch in the HLA-B is also not ideal because the donor's lymphocytes may adequately attack cancer cells expressing high levels of HLA-B alleles but miss the therapeutic treatment of those cancer cells expression low levels of the HLA-B alleles. A donor with a mismatch within the HLA-A allele is ideal because the donor's lymphocytes can attack the mismatched HLA-A allele that is expressed at a high level with little variability by cancer cells. This strategy leads to selecting Donor 2 which mismatches that patient's HLA-A allele at A*03.01.

FIG. 1B depicts assessing a patient's HLA alleles at a time of relapse to inform clinical decision making. At the time of relapse, the patient's cancer cells can be assessed for normal HLA expression (outcome 1, top), loss of the expression of one HLA allele (outcome 2, middle), or reduced expression of HLA alleles (outcome 3, bottom). If the HLA expression by the patient's cancer cells is at an expected level for both alleles, a clinical decision could be to repeat a previous treatment, for example, reinfusing donor lymphocytes from the same donor. This is to bolster the initial treatment, providing a second dose for continued therapeutic effect. If the HLA expression by the patient's leukemic cells is lost for one allele, a clinical decision could be to reinfuse donor lymphocytes from the same donor if the lost allele is not one targeted by the donor's lymphocytes. If, however, the lost allele is one targeted by donor's lymphocytes, these lymphocytes would be less effective following loss of the allele, and a different donor could be identified. If the HLA expression by the patient's cancer cells is reduced, a therapy to increase HLA expression could be administered. Therapies that increase HLA expression include hypomethylating agents such as Azacytidine; Decitabine; DNMT1 inhibitor (Cladribine/Fludarabine/Clofarabine, Procainamide/Procaine); Zebularine; (−)-epigallocatechin-3-gallate; MG98; Hydralazine; RG108; Chlorogenic acid; those described in U.S. Publication Nos. 2011/0218170A1, 2005/0119201, and 2015; compounds that inhibit DNA methyltransferases; compounds that inhibit EZH2 methyltransferase; histone deacetylase (HDAC) inhibitors, ALK and ERBB2 kinase inhibitors (PMC6891198); cytokines and growth factors (e.g., IFN-α, IFN-γ, TNF-α) (doi/pdf/10.1046/j.1365-2567.1997.00258.x); anti-EGFR agents (PMC5635422); (chloroquine+ICI therapy (anti-PD-L1 and anti-CTLA4) (www.frontiersin.org/articles/10.3389/fimmu.2022.844866/full #h6); chemotherapy (e.g., Docetaxel); topoisomerase inhibitors (e.g. etoposide); and microtubule stabilizers (e.g. paclitaxel).

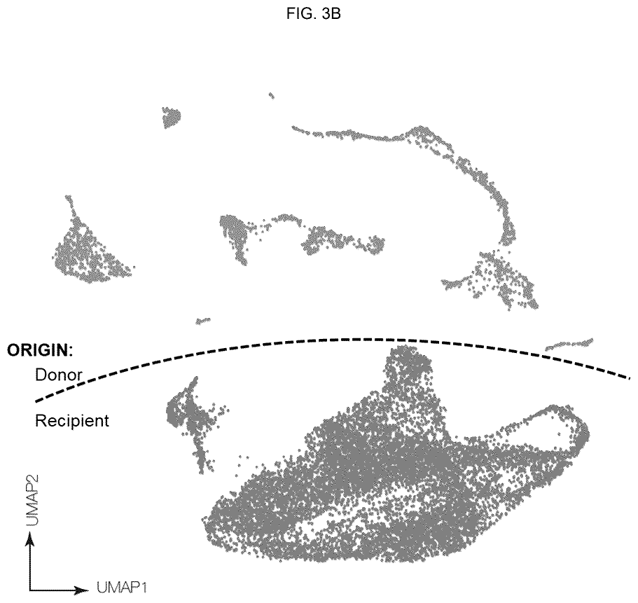

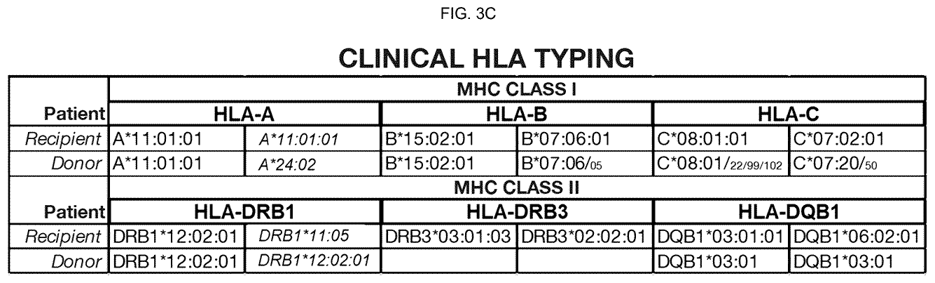

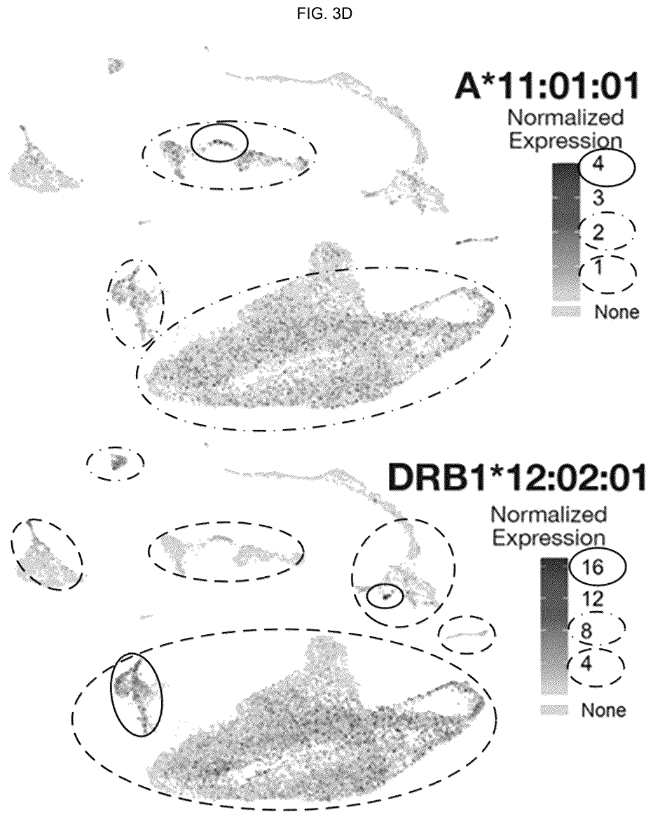

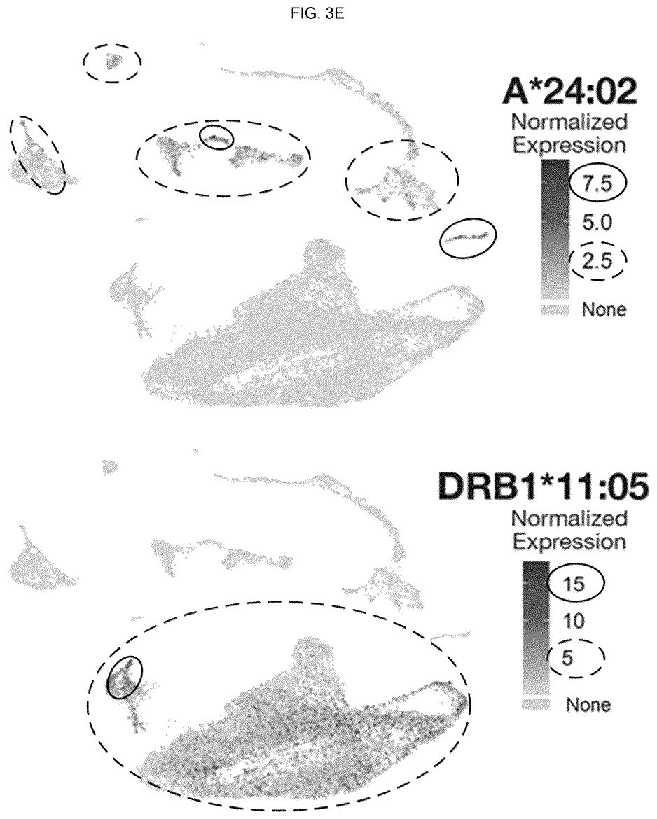

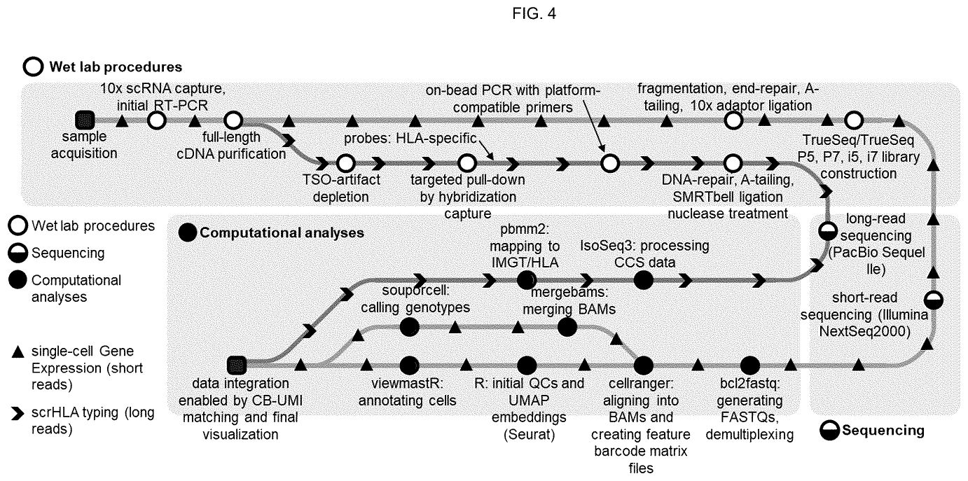

The current disclosure provides these advancements in clinical decision making by providing methods and tools for in-depth sequencing of targeted HLA genes on human chromosome 6 from a single-cell barcoded transcriptome. As indicated, the goal is to identify Human Leukocyte Antigen (HLA) alleles (as of 2025, >42,000 HLA alleles have been identified), as well as examining HLA isoform variations and expression levels, at the single cell level. Disclosed methods can utilize generating a whole transcriptome amplified pool (the cDNA pool), with each cDNA molecule including a cell-barcode (CB) sequence (to link it to a specific cell) and a unique molecular identifier (UMI) sequence (to link transcript counts per each cell). Hybridization capture and enrichment of the transcript(s) of interest can then be performed. This involves the use of one or more of custom-designed biotinylated DNA oligos to specifically hybridize cDNA molecules, custom-designed DNA oligos with modified 3-end nucleotides to block unwanted amplification artifacts, PCR-enrichment of captured library with single-cell platform-specific primers, and indexing the library according to the appropriate downstream sequencing application (e.g., Pacific Biosciences or Oxford Nanopore Technology long reads). Computational tools can be used to identify HLA-genotypes based on publically-available HLA allelic variants using sequenced data from individuals cells. Examples of software that can be used to determine HLA alleles of the subject from next generation sequencing include PHLAT (Bai et al, BMC Genomics 15:325, 2014), HLAreporter (Huang et al, Genome Med. 7:25, 2015), HLAscan (Ka et al. BMC Bioinformatics 18:258, 2017), OptiType (Szolek et al., Bioinformatics 30:3310-3316, 2014), HLA-MA (Messerschmidt et al., Bioinformatics 33:2241-2242, 2017), HLAminer (Warren et al, Genome Med. 4:95, 2012), and ATHLATES (Liu et al., Nucleic Acids Res. 41:e142, 2013). Examples of software that can be used to determine allele copy numbers from next generation sequencing include Sequenza (Favero et al., Ann. Oncol. 26:64-70, 2015) and LOHHLA (loss of heterozygosity in human leukocyte antigen, which specifically evaluates LOH at the HLA locus) (McGranahan et al., Cell 171:1259-1271, 2017). As shown in the FIG. 3B, single-cell RNA-based HLA-typing is able to correctly identify HLA variants in a patient with relapsed acute myeloid leukemia after hematopoietic cell transplantation (HCT), distinguishing individual cells carrying patient-specific HLA genotypes from those carrying donor-specific genotypes, consistent with clinical, DNA-based HLA-typing readily available for transplant patients.

Aspects of the current disclosure are now described with additional detail and options, as follows: (i) Samples for Analysis; (ii) Primers and Adapters for Selecting RNA Types for cDNA Generation; (iii) Hybridization Capture of cDNA; (iv) PCR Artifact Reduction; (v) PCR Amplification and Detection; (vi) Reference Levels & Analysis; (vii) Kits; (viii) Exemplary Embodiments; (ix) Experimental Examples; and (x) Closing Paragraphs. These headings are provided for organizational purposes only and do not limit the scope or interpretation of the disclosure.

### (i) Samples for Analysis

In particular embodiments, a sample for analysis is derived from a bone marrow sample. In particular embodiments, bone marrow aspirates (BMA) are obtained from the pelvic bone of patients and are processed to isolate mononuclear cells using density gradient centrifugation or other appropriate cell sorting techniques (e.g., magnetic bead-based sorting). Any remaining red blood cells (RBCs) can be depleted.

In certain embodiments the sample is a cancer-associated body fluid or tissue. In particular embodiments, the sample is blood sample. The sample may contain a blood fraction (e.g. a serum sample or a plasma sample) or may be whole blood. Suitably, the sample may be circulating cancer cells. The circulating cancer cells may be isolated from a blood sample obtained from the subject. Techniques for collecting samples from a subject are well known in the art. Circulating cancer cells may be isolated from blood samples according to e.g. Nature. 2017 Apr. 26; 545(7655):446-451 or Nat Med. 2017 January; 23(1):114-119.

In particular embodiments, the sample is a “tumor sample,” referring to a sample deriving or obtained from a tumor. The tumor may be a non-solid hematological tumor or a solid tumor. The tumor sample may be a primary tumor sample, tumor-associated lymph node sample or sample from a metastatic site from the subject. Tumor samples and non-cancerous tissue samples can be obtained according to any method known in the art. For example, tumor and non-cancerous samples can be obtained from cancer patients that have undergone resection, or they can be obtained by extraction using a hypodermic needle, by microdissection, or by laser capture. Control (non-cancerous) samples can be obtained, for example, from a cadaveric donor or from a healthy donor.

RNA suitable for downstream cDNA generation can be isolated from cancer cells derived from the sample. For example RNA isolation may be performed using phenol-based extraction. Phenol-based reagents contain a combination of denaturants and RNase inhibitors for cell and tissue disruption and subsequent separation of RNA from contaminants. RNA may further be isolated using solid phase extraction methods (e.g. spin columns) such as QIAGEN RNeasy™ methods. As described in more detail elsewhere herein, isolated RNA may be converted to cDNA for downstream sequencing.

In one aspect more than one sample is obtained and analyzed, for example 2, 3, 4, 5, 6, 7, 8, 9, or 10 samples.

### (ii) Primers and Adapters for Selecting RNA Types for cDNA Generation

Different primers and/or adapters can be used to select different types of RNA for cDNA generation and sequencing. Exemplary types of RNA include small RNA such as a micro RNAs (miRNA), piwi interacting RNA (piRNA), small interfering RNA (siRNA), repeat associated siRNA (rasiRNA), trans-acting siRNA (tasiRNA), CRISPR RNA (crRNA), transfer RNA (tRNA), Promoter-associated RNA (PASR), Transcription stop site associated RNAs, signal recognition particle RNA, transfer-messenger RNA (tmRNA), small nuclear RNA (snRNA), small nucleolar RNA (snoRNA), SmyRNA, small Cajal Body-specific RNA (scaRNA), Guide RNA (gRNA), Spliced leader RNA, ribosomal RNA (rRNA), Telomerase RNA, Ribonuclease P, or a large RNA such as long non-coding RNAs or messenger RNAs, retrotransposons, satellite RNA, virioids, viral genomes or fragments thereof.

In certain examples, systems and methods disclosed herein are used to reduce the sequencing of ribosomal RNAs (rRNAs). rRNAs constitute a majority of the mass of total RNAs present in a cell and constitute a major source of interference in RNA-Seq pipelines. When reducing sequencing of rRNA and enriching for coding sequences, PolyA+selection (e.g., positive selection with Oligo-d(T) beads) and/or ribosomal depletion may also be used.

In certain examples, polyT primers (also known as Oligo-d(T) or Oligo-d(T)20 primers) can be selected to selectively produce cDNA from protein-encoding RNA. In certain examples, random hexamers can be used as primers. Random hexamers are random sequences of six nucleotides that anneal to complementary sites on an RNA and act as primers for cDNA synthesis. Gene-specific primers bind target sequences within an mRNA of interest, allowing amplification of only that region. Particular embodiments can combine use of poly T primers, random hexamers, and/or gene-specific primers.

As is understood by one of ordinary skill in the art, adapters can also be used to target particular types of RNA for cDNA generation or to allow for labeling all types of RNA for non-selective cDNA generation. Useful RNA adapters are described in, for example, US2014/0357528. Adapters which provide priming sequences for both amplification and sequencing of fragments for use with the 454 Life Science GS20 sequencing system are described by F. Cheung, et al. in BMC Genomics 2006, 7:272.

Ligation of RNA adapters to RNA can be achieved using a suitable nucleic acid ligase such as T4 RNA ligase 1 (T4 Rnl1) T4 RNA ligase 2 (T4 Rnl2), T4 RNA ligase 2 truncated (also defined as T4 RNA Ligase 2 1-249) and T4 ligase 2 truncated K227Q (T4 Rnl2tr K227Q), T4 DNA ligase 2 truncated R55K, K227Q (T4 Rnl2tr KQ), T4 DNA ligase, T3 DNA ligase, T7 DNA ligase, E. coli DNA ligase, 9° N™ DNA ligase, Thermus aquaticus DNA ligase, Paramecium bursaria chlorella virus 1 (PBCV-1) ligase, Methanobacterium thermoautotrophicum RNA ligase (Mth ligase), or RtcB family ligases such as E. coli RtcB ligase or variants of these ligases (New England Biolabs, Ipswich, Mass.) that support the complete ligation reaction or at least phosphodiester bond formation between nucleic acid polymers.

Particular embodiments increase the incorporation of adapters into cDNA sequences, which can be used to add synthetic priming sites on targets of interest (to facilitate target amplification), or barcodes and unique molecular identifiers (UMI) to aid the computational processing of resulting sequencing reads for greater accuracy.

In particular embodiments, the barcode or UMI can be any appropriate length and composition that does not negatively affect experimental results. In particular embodiments, the length of the barcode or UMI is based upon the number of cells undergoing analysis. If more distinct barcodes are needed, then barcodes of greater length can be used. If less distinct barcodes are needed, then barcodes of lesser length can be used. In particular embodiments, the barcode or UMI can be 5-100 nucleotides in length. In particular embodiments, the barcode or UMI can be 10-80 nucleotides in length. In particular embodiments, the barcode or UMI can be 10-50 nucleotides in length. In particular embodiments, the barcode or UMI can be 8-30 nucleotides in length. In particular embodiments, the barcode or UMI can be 12-24 nucleotides in length. In particular embodiments, the barcode or UMI can be 16-20 nucleotides in length. In particular embodiments, the barcode or UMI can be 3 nucleotides in length, 4 nucleotides in length, 5 nucleotides in length, 6 nucleotides in length, 7 nucleotides in length, 8 nucleotides in length, 9 nucleotides in length, 10 nucleotides in length, 11 nucleotides in length, 12 nucleotides in length, 13 nucleotides in length, 14 nucleotides in length, 15 nucleotides in length, 16 nucleotides in length, 17 nucleotides in length, 18 nucleotides in length, 19 nucleotides in length, 20 nucleotides in length, 21 nucleotides in length, 22 nucleotides in length, 23 nucleotides in length, 24 nucleotides in length, 25 nucleotides in length, 26 nucleotides in length, 27 nucleotides in length, 28 nucleotides in length, 29 nucleotides in length, 30 nucleotides in length, 31 nucleotides in length, 32 nucleotides in length, 33 nucleotides in length, 34 nucleotides in length, 35 nucleotides in length, 36 nucleotides in length, 37 nucleotides in length, 38 nucleotides in length, 39 nucleotides in length, 40 nucleotides in length, or more.

RNA can be subjected to any form of cDNA generation or sequencing. Reverse Transciptases (RT) are enzymes that perform reverse transcription of RNA into a first strand of cDNA. More processive RT can be used to increase sequence read lengths. In certain examples, the processivity of an RT refers to the ability of an RT to generate a complementary strand of DNA across the full-length of the template RNA. Some RT enzymes (e.g., SuperScript IV (SSIV) achieve this via multiple binding events, whereas others (e.g., MarathonRT), can do so in single binding event. RT with higher processivity synthesize longer cDNA strands than RT with lower processivity. In certain examples, an RT that adds 1,500 nucleotides is considered highly processive or to have high processivity.

Traditionally, RT included Moloney Murine Leukemia Virus RT (M-MLV RT) and Avian Myeloblastosis Virus RT (AMV RT). RT have since been developed that are superior for the generation of longer, or full-length, cDNAs, even at lower temperature ranges. For example, the M-MLV gene was mutated to eliminate the endogenous RNase H activity and this modified enzyme was referred to as Superscript™ II RT (Gibco-BRL). Superscript™ II RNase H-RT (see U.S. Pat. No. 5,244,797) is purified to near homogeneity from E. coli containing the pol gene of M-MLV. An exemplary RT PCR process that employs Superscript™ II RNase H-RT can be found in the Gibco catalog. Briefly, a 20-μl reaction volume can be used for 1-5 μg of total RNA or 50-500 ng of mRNA. The following components are added to a nuclease-free microcentrifuge tube:1 μl Oligo (dT)12-18 (500 μg/ml) 1-5 μg total RNA, sterile, distilled water to 12 μl. The reaction mixture is heated to 70° C. for 10 min and quickly chilled on ice. The contents of the tube are collected by brief centrifugation. To this precipitate is added: 4 pl 5×First Strand Buffer, 2 μl 0.1 M DTT, 1 μl 10 mM dNTP Mix (10 mM each dATP, dGTP, dCTP and dTTP at neutral pH). The contents are mixed gently and incubate at 42° C. for 2 min. Then 1 μl (200 units) of Superscript II™ is added and the reaction mixture is mixed by pipetting gently up and down. This mixture is then incubated for 50 min at 42° C. and then inactivated by heating at 70° C. for 15 min. The cDNA can then be used as a template for amplification in PCR.

In certain embodiments, the RT are thermocycling RT, thereby allowing for amplification of RNA templates in a single reaction. In certain embodiments, the RT are functional at physiologic temperature, thereby allowing for efficient reverse transcription under conditions that reduce the degradation of the RNA template. In certain embodiments, the RT efficiently copy long RNAs in a single turnover, thereby allowing the presently described RT to be used at lower RT concentrations and in single molecule sequencing technologies.

In particular embodiments, the RT is derived from Eubacterium rectale (E.r.) maturase. In certain embodiments, the RT is modified relative to wildtype E.r. maturase. For example, in certain embodiments, the variant includes one or more point mutations, insertion mutations, or deletion mutations, relative to wildtype E.r. maturase. In certain embodiments, the variant includes a fusion protein including E.r. maturase, E.r. maturase mutant, or E.r. maturase domain.

In certain examples, the RT with high processivity is MarathonRT. For more information regarding RT with high processivity based on wildtype E.r. maturase, see US 20210155910 (also published as WO2019005955).

In particular embodiments the E.r. maturase or a variant of E.r. maturase is used in an optimized reaction buffer, wherein the optimized reaction buffer includes Tris at a concentration of 10 mM to 100 mM, KCl at a concentration of 100 mM to 500 mM, MgCl₂ at a concentration of 0.5 mM to 5 mM, DTT at a concentration of 1 mM to 10 mM, and wherein the optimized reaction buffer has a pH of 8 to 8.5. In particular embodiments, the optimized reaction buffer further includes one or more protein stabilizing agents.

In particular embodiments, a selected RT can include a Roseburia intestinalis (R.i.) maturase, or a variant or fragment thereof.

Certain examples can utilize an RT derived from Bacillus stearothermophilus (Geobacillus stearothermophilus), for example, that commercially available as TGIRT (Ingex, LCC, St. Louis, MO) and/or as described in U.S. Pat. No. 7,670,807).

### (iii) Hybridization Capture of cDNA

As indicated, hybridization capture of the cDNA transcript(s) of interest involves the use of custom-designed biotinylated DNA oligos to specifically hybridize cDNA molecules. In particular embodiments, these include a series of 5-prime biotinylated hybridization probes, between 65 and 107 nucleotides in length (e.g., 73 and 99 nucleotides long), that hybridize to all known HLA sequences on human chromosomal region 6p21, with E-val scores>1e-20 after running a BLASTN on the alleles. The alleles belong to the following genes: HLA-A, -B, -C, -E, -F, -G, -H, -J, -K, -L, -T, -V, -W, -Y, -DMA, -DMB, -DOA, -DOB, -DPA1, -DPA2, -DPB1, -DPB2, -DQA1, -DQA2, -DQB1, -DRA, -DRB1, -DRB2, -DRB3, -DRB4, -DRB5, -DRB6, -DRB7, -DRB8, -DRB9, as well as HFE, MICA, MICB, TAP1, and TAP2. Deoxyadenosines (e.g., 2-8 deoxyadenosines) can be further added at the 3-prime end of the oligos to prevent them from acting as primers in subsequent PCRs. These probes are agnostic to the platform used, in the sense that any single cell capture platform capable of generating cell-barcoded whole transcriptome amplicons would be suitable for these probes. The probes hybridize on HLA transcripts and are pull down using commercially available magnetic beads coated with streptavidin. Other appropriate pull down systems may also be used. HLA specific hybridization probes disclosed herein are provided in Table 1.

TABLE 1
HLA Specific Hybridization Probes with /5Biosg/ Leads (5-prime biotin tags) & AAAA Tails (4
consecutive Adenines at the 3-prime tail of the nucleic acid sequence of the probe)

| Name | Hybridizes To: | Sequence (5′-3′) |
|---|---|---|
| PanCls1_a | HLA class 1 transcripts including | /5Biosg/ACCAGGCCTGCAGGGGATGGAACCTTCCAG |
| | HLA-A, -B, -C, -E, -F, -G, -H, -J, -K, | AAGTGGGCGKCTGTGGTGGTRCCTTCTGGASAGGA |
| | -L, -T, -V, -W, and -Y | GCAGAGATACACCTGCCATGTGCAGCATGAGGAAA |
| | | A (SEQ ID NO: 37) |
| PanCls1_b | HLA class 1 transcripts including | /5Biosg/GTYTSCCCAAGCCCCTCACCCTGAGATGGG |
| | HLA-A, -B, -C, -E, -F, -G, -H, -J, -K, | AGCYGTCTTCCCAGCCCACCATCCCCATCGTGGGC |
| | -L, -T, -V, -W, and -Y | ATCATTGCTGGCCTGGTTCTCYTTGGAGCTGTGWAA |
| | | AA (SEQ ID NO: 38) |
| PanCls1_c | HLA class 1 transcripts including | /5Biosg/GGCAGCTGTGGTGGTGCCTTCTGGAGAAGA |
| | HLA-A, -B, -C, -E, -F, -G, -H, -J, -K, | GCAGAGATACACATGCCATGTACAGCATGAGGGGC |
| | -L, -T, -V, -W, and -Y | TGCCRAAGCCCCTCACCCTGAGATGGGAAAA (SEQ |
| | | ID NO: 39) |
| PanCls1_d | HLA class 1 transcripts including | /5Biosg/ACYTGGGGTCCGACGGWCGCCTCMTCCGC |
| | HLA-A, -B, -C, -E, -F, -G, -H, -J, -K, | GGGTATGAACAGTATGCCTACGATGGCAAGGATTAC |
| | -L, -T, -V, -W, and -Y | CTCGCCCTGAACGAGGACCTGCGCTCCTAAAA (SEQ |
| | | ID NO: 40) |
| DMA_a | HLA-DMA | /5Biosg/GCGAGTGGATGATCCAGCAAATAGGGCCAA |
| | | AACTTGATGGGAAAATCCCGGTGTCCAGAGGGTTTC |
| | | CTATCGCTGAAGTGTTCACGCTGAAGCCCCTGAAAA |
| | | (SEQ ID NO: 41) |
| DMA_b | HLA-DMA | /5Biosg/GAGTTTGGCAAGCCCAACACTTTGGTCTGTT |
| | | TTGTCAGTAATCTCTTCCCACCCATGCTGACAGTGA |
| | | ACTGGCAGCATCATTCCRTCCCTGTGGAAGGAAAA |
| | | (SEQ ID NO: 42) |
| DMB_a | HLA-DMB | /5Biosg/CCCCTCTTACGGGGACACTTACACCTGTGTG |
| | | GTAGAGCACAYTGGGGCTCCTGAGCCCATCCTTCG |
| | | GGACTGGACACCTGGGCTGTCCCAAAA (SEQ ID NO: |
| | | 43) |
| DMB_b | HLA-DMB | /5Biosg/CCATGCAGACCCTGAAGGTTTCTGTGTCTGC |
| | | AGTGACTCTGGGCCTGGGCCTCATCATCTTCTCTCT |
| | | TGGTGTGATCAGCTGGCGGAGAGCTGGCCAAAA |
| | | (SEQ ID NO: 44) |
| DOA_a | HLA-DOA | /5Biosg/GGGCCAGCCCAACATCCTCATCTGCATCGT |
| | | GGACAACATCTTCCCCCCTGTGATCAATATCACCTG |
| | | GCTRCGCAACGGCCAAACTGTCACTGAGGGAGAAA |
| | | A (SEQ ID NO: 45) |
| DOA_b | HLA-DOA | /5Biosg/TGGCCCAGACCAGCTTCTATTCCCAGCCTGA |
| | | CCATTTGTTCCGCAAGTTCCACTACCTGCCCTTCGT |
| | | GCCCTCAGCCGAGGACGTCTATGACTGCCAAAAA |
| | | (SEQ ID NO: 46) |
| DOB_a | HLA-DOB | /5Biosg/GGGATGTTTGTGGCATTGACCAAGCTGGGG |
| | | CAGCCAGATGCTGAGCAGTGGAACAGCCGGCTGGA |
| | | TCTCTTGGARAGGAGCAGACAGGCCGTGGAAAA |
| | | (SEQ ID NO: 47) |
| DOB_b | HLA-DOB | /5Biosg/ATGGGGTCTGTAGACACAACTACAGGCTGG |
| | | GCGCACCCTTCACTGTGGGGAGAAAAGTGCAACCA |
| | | GAGGTGACAGTGTACCCAGAGAGGACCCCAAAA |
| | | (SEQ ID NO: 48) |
| DPA_a | HLA-DPA1 and -DPA2 | /5Biosg/CCAGTGCTCAACGTCACRTGGCTGTGCAAY |
| | | GGGGAGCYGGTCACTGAGGGTGTCGCTGAGAGCCT |
| | | CTTCCTGCCCAGAACAGATTACAGCTAAAA (SEQ ID |
| | | NO: 49) |
| DPA_b | HLA-DPA1 and -DPA2 | /5Biosg/TCCACAAGTTCCATTACCTGACCTTTGTGCC |
| | | CTCAGCAGAGGACKTCTATGACTGCAGGGTGGAGC |
| | | ACTGGGGCTTGGACCAGCCGCTCCTAAAA (SEQ ID |
| | | NO: 50) |
| DQA_a | HLA-DQA1 and -DQA2 | /5Biosg/AGGTGGAGCACTGGGGCCTGGAYSAGCCTC |
| | | TTCTGAAACACTGGGAGCCTGAGATTCCAGCMCCTA |
| | | TGTCAGAGCTCACAGAGACTKTGGTCTGYAAAA |
| | | (SEQ ID NO: 51) |
| DQA_b | HLA-DQA1 and -DQA2 | /5Biosg/RCCCTGGGRTTGTCTGTGGGCCTCRTGGGC |
| | | ATTGTGGTGGGCACTGTCTTCATCATCCRAGGCCTG |
| | | CGTTCAGTTGGTGCTTCCAGACACCARGGGAAAA |
| | | (SEQ ID NO: 52) |
| DQB/DPB_a | HLA-DPB1, -DPB2, and -DPQB1 | /5Biosg/GGGCCCYTGCAGCACCACAACCTGCTTGTC |
| | | TGCCACGTGACRGATTTCTACCCAGGCAGCATTCAA |
| | | GTCCGATGGTTCCTGAATGGACAGGAAAA (SEQ ID |
| | | NO: 53) |
| DQB/DPB_b | HLA-DPB1, -DPB2, and -DPQB1 | /5Biosg/AGGAAACAGCTGGGGTCGTGTCCACCAACC |
| | | TGATCCGTAATGGAGACTGGACCTTCCAGATCCTGG |
| | | TGATGCTGGAAATGACCCCCCAGCAAAAA (SEQ ID |
| | | NO: 54) |
| DQB/DPB_c | HLA-DPB1, -DPB2, and -DPQB1 | /5Biosg/TCCCCATCCAGGACAGAGGCCCTCAACCAC |
| | | CACAACCTGCTGRTCTGCTCRGTGACAGATTTCTAT |
| | | CCAGSCCAGATCAAAGTCCGGTGGTTTCGGAAAA |
| | | (SEQ ID NO: 55) |
| DQB/DPB_d | HLA-DPB1, -DPB2, and -DPQB1 | /5Biosg/AATGRYCAGGAGGAGACARCYGGCGTTGTG |
| | | TCCACCCCCCTTATTAGGAAYGGTGACTGGACCTTC |
| | | CAGATCCTGGTGATGCTGGAAATGACTCCCCAAAAA |
| | | (SEQ ID NO: 56) |
| DRA_a | HLA-DRA | /5Biosg/GGGAAGACCACCTTTTCCGCAAGTTCCACTA |
| | | TCTCCCCTTCCTGCCCTCAACTGAGGACGTTTACGA |
| | | CTGCAGGGTGGAGCACTGGGGCTTGGAAAA (SEQ |
| | | ID NO: 57) |
| DRA_b | HLA-DRA | /5Biosg/ATGAGCCTCTTCTCAAGCACTGGGAGTTTGA |
| | | TGCTCCAAGCCCTCTCCCAGAGACTACAGAGAAYGT |
| | | GGTGTGTGCCCTGGGCCTGACTGTGGGAAAA (SEQ |
| | | ID NO: 58) |
| DRB_a | HLA-DRB1, -DRB2, -DRB3, | /5Biosg/TKCAAAGACCCAGCCCCTGCAGCACCACAA |
| | -DRB4, -DRB5, -DRB6, -DRB7, | CCTCCTGGTCTGYTCTGTGARTGGTTTCTATCCAGG |
| | -DRB8, and -DRB9 | CAGCATTGAAGTCAGGTGGTTCCKGAAYAAAA (SEQ |
| | | ID NO: 59) |
| DRB_b | HLA-DRB1, -DRB2, -DRB3, | /5Biosg/RGCCAGGAAGAGAAGRCTGGGGTGGTGTCC |
| | -DRB4, -DRB5, -DRB6, -DRB7, | ACRGGCCTGATCCASAATGGAGACTGGACCTTCCA |
| | -DRB8, and -DRB9 | GACCCTGGTGATGCTGGAAACAGTTCCTCGGAAAA |
| | | (SEQ ID NO: 60) |
| DRB_c | HLA-DRB1, -DRB2, -DRB3, | /5Biosg/GCGGAGCTGGAGCGGAGAAGAGTCCAGGA |
| | -DRB4, -DRB5, -DRB6, -DRB7, | ATRCAAACAGCCAGAAGAACCTCTTGGGCTGCTTGC |
| | -DRB8, and -DRB9 | GGGGTCTGTTGGACACCTACTGCAGACAAAAA (SEQ |
| | | ID NO: 61) |
| HFE_a | HFE | /5Biosg/GCCCTGACACACTGGATTGGAGAGCAGCAG |
| | | AACCCAGGGCCTGGCCCACCAAGCTGGAGTGGGAA |
| | | AGGCACAAGATTCGGGCCAGGCAGAACAGGAAAA |
| | | (SEQ ID NO: 62) |
| HFE_b | HFE | /5Biosg/GCCTACCTGGAGAGGGACTGCCCTGCACAG |
| | | CTGCAGCAGTTGCTGGAGCTGGGGAGAGGTGTTTT |
| | | GGACCAACAAGTGCCTCCTTTGGTGAAGGTGAAAAA |
| | | (SEQ ID NO: 63) |
| MICAB_a | MICA and MICB | /5Biosg/AGTCAGGGTTTCTYRCTGAGGTACATCTGGA |
| | | TGGTCAGCCCTTCCTGCGCTRTGACAGGCAGAAAY |
| | | GCAGGGCAAAA (SEQ ID NO: 64) |
| MICAB_b | MICA and MICB | /5Biosg/AAAGCCCCAGGGACAGTGGGCAGAAGATGT |
| | | CCTGGGAAATAAGACATGGGACAGAGAGACCAGRG |
| | | ACTTGACAGGGAAAAA (SEQ ID NO: 65) |
| TAP1_a | TAP1 | /5Biosg/TGGGGATCAGTGTCCCTCACCATGGTCACC |
| | | CTGRTCACCCTGCCTCTGCTTTTCCTTCTGCCCAAG |
| | | AAGGTGGGAAAATGGTACCAGTTGCTGGAAAA (SEQ |
| | | ID NO: 66) |
| TAP1_b | TAP1 | /5Biosg/AAGTGCAGGTGCGGGAATCTCTGGCAAAGT |
| | | CCAGCCAGGTGGCCATTGAGGYYCTGTCGGCCATG |
| | | CCTACAGTTCGAAGCTTTGCCAACGAGGAGGAAAA |
| | | (SEQ ID NO: 67) |
| TAP2_a | TAP2 | /5Biosg/GCCGTTGGAGGGCTGCAGACCGTTCGCAGT |
| | | TTTGGGGCCGAGGAGCATGAAGTCTGTCGCTATAAA |
| | | GAGGCCCTTGAACAATGTCGGCAGCAAAA (SEQ ID |
| | | NO: 68) |
| TAP2_b | TAP2 | /5Biosg/TGTATTGGCGGAGAGACCTGGAACGCGCCT |
| | | TGTACCTGCTCRTAAGGAGGGTGCTGCACTTGGGK |
| | | GTGCAGATGCTGATGCTGAGCTGTGGAAAA (SEQ ID |
| | | NO: 69) |

The disclosure includes the sequences in Table 1 lacking the depicted “/5Biosg/” and “AAAA”. Further, as is understood by one of ordinary skill in the art, following the degenerate nucleotide notation: R=A/G, Y=C/T, M=A/C, K=G/T, S=C/G, W=NT, B=C/G/T, D=AG/T, H=A/C/T, V=A/C/G, and N=A/C/G/T.

In certain examples, probes can be removed if particular HLA alleles are not of interest in an analysis. For example, in certain examples, a set of hybridization probes is selected so that the set hybridizes to MHC Class I molecules. In particular embodiments, a set of hybridization probes is selected so that the set hybridizes to HLA-A, HLA-B, and HLA-C. In particular embodiments, a set of hybridization probes is selected so that the set hybridizes to HLA-E, HLA-F, HLA-G, MICA, MICB, and HFE, In particular embodiments, a set of hybridization probes is selected so that the set hybridizes to HLA-A, HLA-B, HLA-C, HLA-E, HLA-F, HLA-G, MICA, MICB, and HFE.

In certain examples, a set of hybridization probes is selected so that the set hybridizes to MHC class II molecules. In particular embodiments, a set of hybridization probes is selected so that the set hybridizes to HLA-DR, HLA-DQ, and HLA-DP, In particular embodiments, a set of hybridization probes is selected so that the set hybridizes to HLA-DM and HLA-DO, In particular embodiments, a set of hybridization probes is selected so that the set hybridizes to HLA-DR, HLA-DQ, HLA-DP, HLA-DM and HLA-DO.

In particular embodiments, a set of hybridization probes is selected so that the set hybridizes to HLA-A, HLA-B, HLA-C, HLA-DR, HLA-DQ, and HLA-DP.

In certain examples, the panel excludes PanCls1_a. In certain examples, the panel excludes PanCls1_b. In certain examples, the panel excludes PanCls1_c. In certain examples, the panel excludes PanCls1_d. In certain examples, the panel excludes DMA_a. In certain examples, the panel excludes DMA_b. In certain examples, the panel excludes DMB_a. In certain examples, the panel excludes DMB_b. In certain examples, the panel excludes DOA_a. In certain examples, the panel excludes DOA_b. In certain examples, the panel excludes DOB_a. In certain examples, the panel excludes DOB_b. In certain examples, the panel excludes DPA_a. In certain examples, the panel excludes DPA_b. In certain examples, the panel excludes DQA_a. In certain examples, the panel excludes DQA_b. In certain examples, the panel excludes DQB/DPB_a. In certain examples, the panel excludes DQB/DPB_b. In certain examples, the panel excludes DQB/DPB_c. In certain examples, the panel excludes DQB/DPB_d. In certain examples, the panel excludes DRA_a. In certain examples, the panel excludes DRA_b. In certain examples, the panel excludes DRB_a. In certain examples, the panel excludes DRB_b. In certain examples, the panel excludes DRB_c. In certain examples, the panel excludes HFE_a. In certain examples, the panel excludes HFE_b. In certain examples, the panel excludes MICAB_a. In certain examples, the panel excludes MICAB_b. In certain examples, the panel excludes TAP1_a. In certain examples, the panel excludes TAP1_b. In certain examples, the panel excludes TAP2_a. In certain examples, the panel excludes TAP2_b.

In certain examples, the panel excludes two probes, wherein one probe of the pair is PanCls1_a. In certain examples, the panel excludes two probes, wherein one probe of the pair is PanCls1_b. In certain examples, the panel excludes two probes, wherein one probe of the pair is PanCls1_c. In certain examples, the panel excludes two probes, wherein one probe of the pair is PanCls1_d. In certain examples, the panel excludes two probes, wherein one probe of the pair is DMA_a. In certain examples, the panel excludes two probes, wherein one probe of the pair is DMA_b. In certain examples, the panel excludes two probes, wherein one probe of the pair is DMB_a. In certain examples, the panel excludes two probes, wherein one probe of the pair is DMB_b. In certain examples, the panel excludes two probes, wherein one probe of the pair is DOA_a. In certain examples, the panel excludes two probes, wherein one probe of the pair is DOA_b. In certain examples, the panel excludes two probes, wherein one probe of the pair is DOB_a. In certain examples, the panel excludes two probes, wherein one probe of the pair is DOB_b. In certain examples, the panel excludes two probes, wherein one probe of the pair is DPA_a. In certain examples, the panel excludes two probes, wherein one probe of the pair is DPA_b. In certain examples, the panel excludes two probes, wherein one probe of the pair is DQA_a. In certain examples, the panel excludes two probes, wherein one probe of the pair is DQA_b. In certain examples, the panel excludes two probes, wherein one probe of the pair is DQB/DPB_a. In certain examples, the panel excludes two probes, wherein one probe of the pair is DQB/DPB_b. In certain examples, the panel excludes two probes, wherein one probe of the pair is DQB/DPB_c. In certain examples, the panel excludes two probes, wherein one probe of the pair is DQB/DPB_d. In certain examples, the panel excludes two probes, wherein one probe of the pair is DRA_a. In certain examples, the panel excludes two probes, wherein one probe of the pair is DRA_b. In certain examples, the panel excludes two probes, wherein one probe of the pair is DRB_a. In certain examples, the panel excludes two probes, wherein one probe of the pair is DRB_b. In certain examples, the panel excludes two probes, wherein one probe of the pair is DRB_c. In certain examples, the panel excludes two probes, wherein one probe of the pair is HFE_a. In certain examples, the panel excludes two probes, wherein one probe of the pair is HFE_b. In certain examples, the panel excludes two probes, wherein one probe of the pair is MICAB_a. In certain examples, the panel excludes two probes, wherein one probe of the pair is MICAB_b. In certain examples, the panel excludes two probes, wherein one probe of the pair is TAP1_a. In certain examples, the panel excludes two probes, wherein one probe of the pair is TAP1_b. In certain examples, the panel excludes two probes, wherein one probe of the pair is TAP2_a. In certain examples, the panel excludes two probes, wherein one probe of the pair is TAP2_b.

In certain examples, the panel excludes two probes including DMB_b and MICAB_b. In certain examples, the panel excludes two probes including PanCls1_c and HFE_a. In certain examples, the panel excludes two probes including DMA_a and TAP2_b. In certain examples, the panel excludes two probes including DOB_b and HFE_b. In certain examples, the panel excludes two probes including PanCls1_a and DQA_a. In certain examples, the panel excludes two probes including DOA_a and DQB/DPB_b. In certain examples, the panel excludes two probes including DQB/DPB_d and TAP1_a. In certain examples, the panel excludes two probes including DOA_b and DRA_bin certain examples, the panel excludes two probes including DQA_b and DRB_c. In certain examples, the panel excludes two probes including DPA_a and DQB/DPB_d.

In certain examples, the panel excludes three probes, wherein one probe of the pair is PanCls1_a. In certain examples, the panel excludes three probes, wherein one probe of the pair is PanCls1_b. In certain examples, the panel excludes three probes, wherein one probe of the pair is PanCls1_c. In certain examples, the panel excludes three probes, wherein one probe of the pair is PanCls1_d. In certain examples, the panel excludes three probes, wherein one probe of the pair is DMA_a. In certain examples, the panel excludes three probes, wherein one probe of the pair is DMA_b. In certain examples, the panel excludes three probes, wherein one probe of the pair is DMB_a. In certain examples, the panel excludes three probes, wherein one probe of the pair is DMB_b. In certain examples, the panel excludes three probes, wherein one probe of the pair is DOA_a. In certain examples, the panel excludes three probes, wherein one probe of the pair is DOA_b. In certain examples, the panel excludes three probes, wherein one probe of the pair is DOB_a. In certain examples, the panel excludes three probes, wherein one probe of the pair is DOB_b. In certain examples, the panel excludes three probes, wherein one probe of the pair is DPA_a. In certain examples, the panel excludes three probes, wherein one probe of the pair is DPA_b. In certain examples, the panel excludes three probes, wherein one probe of the pair is DQA_a. In certain examples, the panel excludes three probes, wherein one probe of the pair is DQA_b. In certain examples, the panel excludes three probes, wherein one probe of the pair is DQB/DPB_a. In certain examples, the panel excludes three probes, wherein one probe of the pair is DQB/DPB_b. In certain examples, the panel excludes three probes, wherein one probe of the pair is DQB/DPB_c. In certain examples, the panel excludes three probes, wherein one probe of the pair is DQB/DPB_d. In certain examples, the panel excludes three probes, wherein one probe of the pair is DRA_a. In certain examples, the panel excludes three probes, wherein one probe of the pair is DRA_b. In certain examples, the panel excludes three probes, wherein one probe of the pair is DRB_a. In certain examples, the panel excludes three probes, wherein one probe of the pair is DRB_b. In certain examples, the panel excludes three probes, wherein one probe of the pair is DRB_c. In certain examples, the panel excludes three probes, wherein one probe of the pair is HFE_a. In certain examples, the panel excludes three probes, wherein one probe of the pair is HFE_b. In certain examples, the panel excludes three probes, wherein one probe of the pair is MICAB_a. In certain examples, the panel excludes three probes, wherein one probe of the pair is MICAB_b. In certain examples, the panel excludes three probes, wherein one probe of the pair is TAP1_a. In certain examples, the panel excludes three probes, wherein one probe of the pair is TAP1_b. In certain examples, the panel excludes three probes, wherein one probe of the pair is TAP2_a. In certain examples, the panel excludes three probes, wherein one probe of the pair is TAP2_b.

In certain examples, the panel excludes three probes including PanCls1_a, DMB_b and MICAB_b. In certain examples, the panel excludes three probes including PanCls1_c, DPA_a, and HFE_a. In certain examples, the panel excludes three probes including DMA_a, DQA_b, and DRB_b. In certain examples, the panel excludes three probes including DOB_b, DQB/DPB_d, and HFE_b. In certain examples, the panel excludes three probes including PanCls1_d, DMA_b, and DQA_a. In certain examples, the panel excludes three probes including DOA_a, DQB/DPB_b, and TAP2_a. In certain examples, the panel excludes three probes including DOB_a, DQB/DPB_d and TAP1_a. In certain examples, the panel excludes three probes including DOA_b, DRA_b, and MICAB_a. In certain examples, the panel excludes three probes including DQA_b, DQB/DPB_a, and DRB_c. In certain examples, the panel excludes three probes including DPA_b, DQB/DPB_d, and TAP1_b.

In certain examples, the panel excludes four probes, wherein one probe of the pair is PanCls1_a. In certain examples, the panel excludes four probes, wherein one probe of the pair is PanCls1_b. In certain examples, the panel excludes four probes, wherein one probe of the pair is PanCls1_c. In certain examples, the panel excludes four probes, wherein one probe of the pair is PanCls1_d. In certain examples, the panel excludes four probes, wherein one probe of the pair is DMA_a. In certain examples, the panel excludes four probes, wherein one probe of the pair is DMA_b. In certain examples, the panel excludes four probes, wherein one probe of the pair is DMB_a. In certain examples, the panel excludes four probes, wherein one probe of the pair is DMB_b. In certain examples, the panel excludes four probes, wherein one probe of the pair is DOA_a. In certain examples, the panel excludes four probes, wherein one probe of the pair is DOA_b. In certain examples, the panel excludes four probes, wherein one probe of the pair is DOB_a. In certain examples, the panel excludes four probes, wherein one probe of the pair is DOB_b. In certain examples, the panel excludes four probes, wherein one probe of the pair is DPA_a. In certain examples, the panel excludes four probes, wherein one probe of the pair is DPA_b. In certain examples, the panel excludes four probes, wherein one probe of the pair is DQA_a. In certain examples, the panel excludes four probes, wherein one probe of the pair is DQA_b. In certain examples, the panel excludes four probes, wherein one probe of the pair is DQB/DPB_a. In certain examples, the panel excludes four probes, wherein one probe of the pair is DQB/DPB_b. In certain examples, the panel excludes four probes, wherein one probe of the pair is DQB/DPB_c. In certain examples, the panel excludes four probes, wherein one probe of the pair is DQB/DPB_d. In certain examples, the panel excludes four probes, wherein one probe of the pair is DRA_a. In certain examples, the panel excludes four probes, wherein one probe of the pair is DRA_b. In certain examples, the panel excludes four probes, wherein one probe of the pair is DRB_a. In certain examples, the panel excludes four probes, wherein one probe of the pair is DRB_b. In certain examples, the panel excludes four probes, wherein one probe of the pair is DRB_c. In certain examples, the panel excludes four probes, wherein one probe of the pair is HFE_a. In certain examples, the panel excludes four probes, wherein one probe of the pair is HFE_b. In certain examples, the panel excludes four probes, wherein one probe of the pair is MICAB_a. In certain examples, the panel excludes four probes, wherein one probe of the pair is MICAB_b. In certain examples, the panel excludes four probes, wherein one probe of the pair is TAP1_a. In certain examples, the panel excludes four probes, wherein one probe of the pair is TAP1_b. In certain examples, the panel excludes four probes, wherein one probe of the pair is TAP2_a. In certain examples, the panel excludes four probes, wherein one probe of the pair is TAP2_b.

In certain examples, the panel excludes four probes including, DMB_b, DQA_a, MICAB_b, andTAP2_a. In certain examples, the panel excludes four probes including PanCls1_c, DQB/DPB_c, DRA_a, and HFE_a. In certain examples, the panel excludes four probes including PanCls1_b, DMA_a, DQA_b, and DRB_b. In certain examples, the panel excludes four probes including PanCls1_a, DOB_a, DPA_a, and HFE_b. In certain examples, the panel excludes four probes including PanCls1_d, DMA_b, DOA_a, and HFE_a. In certain examples, the panel excludes four probes including DQB/DPB_a, DRB_a, MICAB_a, and TAP2_a. In certain examples, the panel excludes four probes including PanCls1_a, DOA_b, DOB_b, and TAP1_b. In certain examples, the panel excludes four probes including DMB_a, DOA_b, DRA_b, and MICAB_a. In certain examples, the panel excludes four probes including DQB/DPB_b, DRB_c, DQA_b, and TAP2_b. In certain examples, the panel excludes four probes including PanCls1_c, DPA_b, DQB/DPB_d, and TAP1_a.

In certain examples, the panel excludes five probes, wherein one probe of the pair is PanCls1_a. In certain examples, the panel excludes five probes, wherein one probe of the pair is PanCls1_b. In certain examples, the panel excludes five probes, wherein one probe of the pair is PanCls1_c. In certain examples, the panel excludes five probes, wherein one probe of the pair is PanCls1_d. In certain examples, the panel excludes five probes, wherein one probe of the pair is DMA_a. In certain examples, the panel excludes five probes, wherein one probe of the pair is DMA_b. In certain examples, the panel excludes five probes, wherein one probe of the pair is DMB_a. In certain examples, the panel excludes five probes, wherein one probe of the pair is DMB_b. In certain examples, the panel excludes five probes, wherein one probe of the pair is DOA_a. In certain examples, the panel excludes five probes, wherein one probe of the pair is DOA_b. In certain examples, the panel excludes five probes, wherein one probe of the pair is DOB_a. In certain examples, the panel excludes five probes, wherein one probe of the pair is DOB_b. In certain examples, the panel excludes five probes, wherein one probe of the pair is DPA_a. In certain examples, the panel excludes five probes, wherein one probe of the pair is DPA_b. In certain examples, the panel excludes five probes, wherein one probe of the pair is DQA_a. In certain examples, the panel excludes five probes, wherein one probe of the pair is DQA_b. In certain examples, the panel excludes five probes, wherein one probe of the pair is DQB/DPB_a. In certain examples, the panel excludes five probes, wherein one probe of the pair is DQB/DPB_b. In certain examples, the panel excludes five probes, wherein one probe of the pair is DQB/DPB_c. In certain examples, the panel excludes five probes, wherein one probe of the pair is DQB/DPB_d. In certain examples, the panel excludes five probes, wherein one probe of the pair is DRA_a. In certain examples, the panel excludes five probes, wherein one probe of the pair is DRA_b. In certain examples, the panel excludes five probes, wherein one probe of the pair is DRB_a. In certain examples, the panel excludes five probes, wherein one probe of the pair is DRB_b. In certain examples, the panel excludes five probes, wherein one probe of the pair is DRB_c. In certain examples, the panel excludes five probes, wherein one probe of the pair is HFE_a. In certain examples, the panel excludes five probes, wherein one probe of the pair is HFE_b. In certain examples, the panel excludes five probes, wherein one probe of the pair is MICAB_a. In certain examples, the panel excludes five probes, wherein one probe of the pair is MICAB_b. In certain examples, the panel excludes five probes, wherein one probe of the pair is TAP1_a. In certain examples, the panel excludes five probes, wherein one probe of the pair is TAP1_b. In certain examples, the panel excludes five probes, wherein one probe of the pair is TAP2_a. In certain examples, the panel excludes five probes, wherein one probe of the pair is TAP2_b.

In certain examples, the panel excludes five probes including DMB_b, DQA_a, DRB_a, HFE_a, and MICAB_b. In certain examples, the panel excludes five probes including PanCls1_b, DMA_a, DQB/DPB_c, DRA_a, and HFE_b. In certain examples, the panel excludes five probes including DMA_b, DOB_b, DQB/DPB_a, DRB_b, and TAP1_a. In certain examples, the panel excludes five probes including PanCls1_d, DMB_a, DOB_a, DPA_a, and HFE_b. In certain examples, the panel excludes five probes including DMB_a, DOA_a, DPA_b, DQA_a, and TAP2_b. In certain examples, the panel excludes five probes including PanCls1_b, DQB/DPB_a, DRA_b, MICAB_a, and TAP1_b. In certain examples, the panel excludes five probes including PanCls1_c, DMB_a, DOA_b, DQB/DPB_b, and TAP1_a. In certain examples, the panel excludes five probes including DOA_b, DOB_b, DQA_b, DQB/DPB_d, and TAP2_a. In certain examples, the panel excludes five probes including PanCls1_b, DMA_b, DRB_c, HFE_a, and TAP2_a. In certain examples, the panel excludes five probes including DMB_b, DPA_a, DQB/DPB_a, DRA_a, and MICAB_b.

Various appropriate hybridization conditions can be used. For example, a hybridization mix can be heated at 95° C. for 30 s followed by a rapid ramp down to 65° C. which is held for 4 hours and commercial wash buffers may be applied (for example, the xGen™ kit).

Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. Beads such as streptavidin beads can be used to capture probes bound to their complementary targets.

### (iv) PCR Artifact Reduction

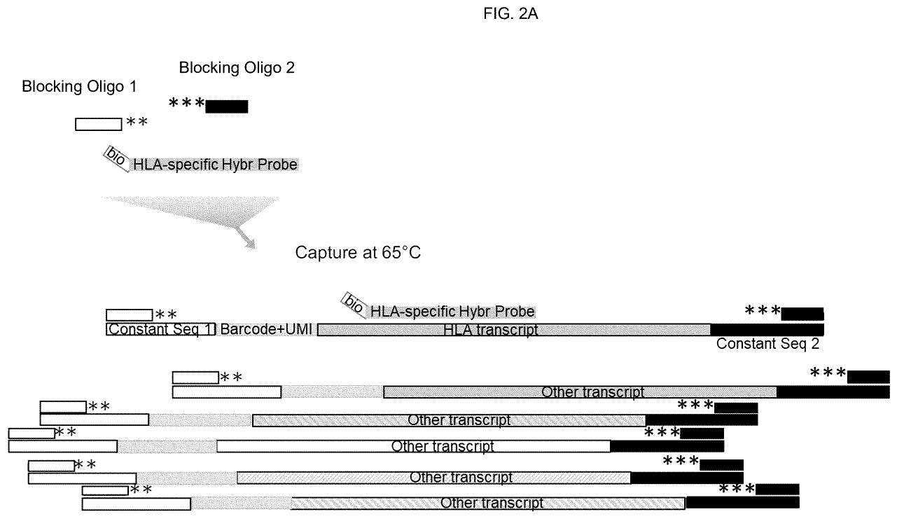

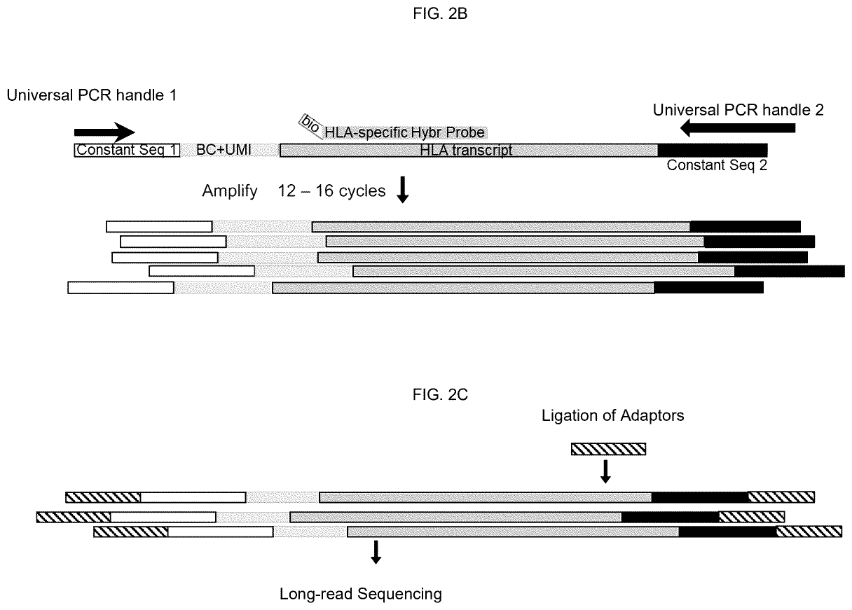

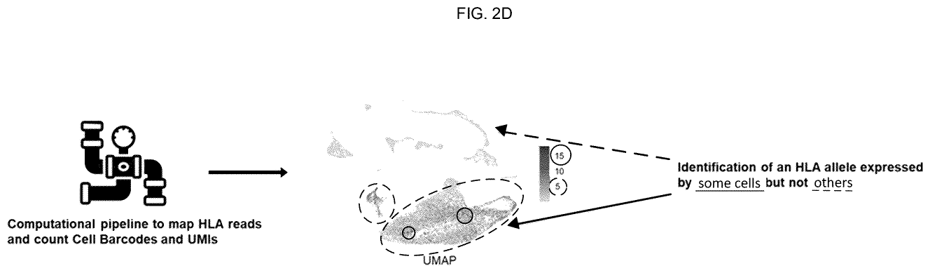

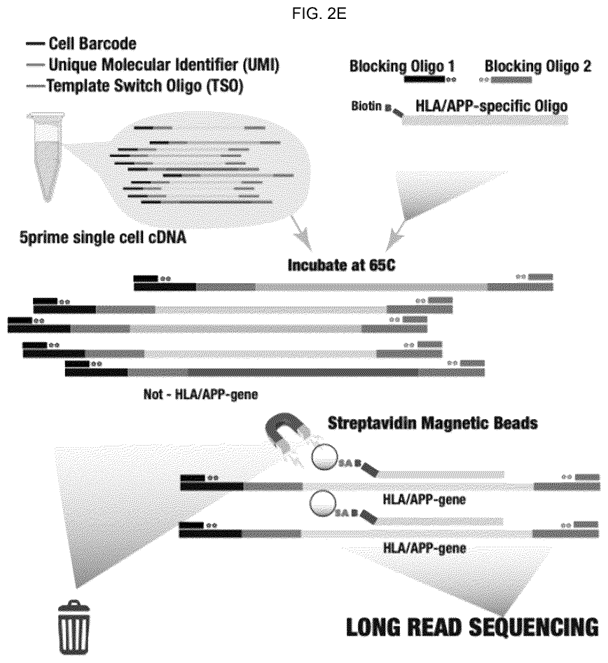

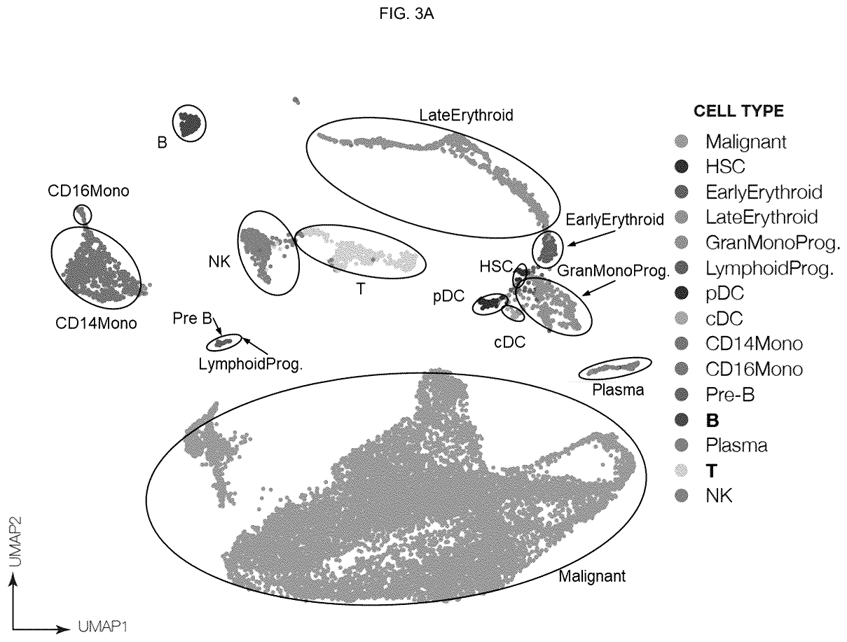

FIGS. 2A-2C show that particular embodiments utilize blocking oligos specific to a given single-cell capture platform (commercially available examples include: 10× genomics, BD Rhapsody, and Dolomite Bio). In these embodiments, the blocking oligos cover up the constant regions on the ends of a captured cDNA molecule. Specifically, where the template switch oligo (TSO) and Poly(dT) constant sequences are located, but also where the highly variable cell barcode and/or unique molecular identifier (CB/UMI) are located. One of the cDNA strands is blocked to prevent it from hybridizing with its complement from another molecule during a PCR reaction and creating the so-called “daisy chain” effect. To overcome the challenge of having a quasi-random set of nucleotides (for the CB/UMIs) inside the constant regions, as it is the case with cDNA captured using the popular 10× Genomics platform, a sequence of deoxyinosine nucleotides acting as “universal base” which, to a large degree, can base pair with any of the 4 classical DNA bases can be used. The oligos can be further modified at their 3-prime end by adding a phosphoramidite spacer to prevent them from extending (i.e., from acting as primers) in subsequent PCRs. Table 2 provides examples of blockers designed for the 10× Genomics platform: for the 5-prime capture chemistry (name starts with “5”) and for the 3-prime capture chemistry (name starts with “3”). Table 3 provides PCR primer oligos that were used. Table 4 provides mutation-specific and fusion-specific hybridization probes.

TABLE 2
Blocking Oligos Designed for the 10x Genomics Platform

| Name | Sequence (5′-3′) |
|---|---|
| 5TSO_blocker | CTACACGACGCTCTTCCGATCTIIIIIIIIIIIIIIIIIIIIIIIIIITTTCTTATATGGG/ |
| | 3SpC3/ (SEQ ID NO: 34) |
| 5PolyA_blocker | AAAAAAAAAAAAAAAAAAAAGTACTCTGCGTTGATACCACTGCTT/ |
| | 3SpC3/ (SEQ ID NO: 35) |
| 3TSO_blocker | AAGCAGTGGTATCAACGCAGAGTACATGGG/3SpC3/ (SEQ ID NO: 70) |
| 3PolyA_blocker | AAAAAAAAAAAAAAAAAAAAIl AGATCGGAAGAGCG |
| | TCGTGTAG/3SpC3/ (SEQ ID NO: 36) |

TABLE 3

| PCR Primer Oligos | |
|---|---|
| Name | Sequence (5′-3′) |
| cDNA_PN_2000089_1 | CTACACGACGCTCTTCCGATCT (SEQ ID NO: 71) |
| cDNA_PN_2000089_2 | AAGCAGTGGTATCAACGCAGAG (SEQ ID NO: 72) |
| R1_10x5_AA0272 | CTACACGACGCTCTTCCGATCT (SEQ ID NO: 71) |
| R2_10x5_dUBio_AA0273 | /5Biosg/UAUAAGCAGTGGTATCAACGCAGAG (SEQ ID NO: 73) |
| Venus_10x3_dUBio_AA0365 | /5Biosg/UAUCTACACGACGCTCTTCCGATCT (SEQ ID NO: 74) |
| Mars_10x3_AA0366 | AAGCAGTGGTATCAACGCAGAG (SEQ ID NO: 72) |
| PTPN11_rev_A227G | ATTTAAGCTCAATGACATCTCCATTCTTCTCTTTTA (SEQ ID NO: 75) |
| IDH1_rev_C394T | TTATCTCTACTTTTCCAGGCCCAGG (SEQ ID NO: 76) |
| TP53_rev_G743A | TCAAAGCTGTTCCGTCCCAGTAGATTA (SEQ ID NO: 77) |

TABLE 4
Mutation-Specific and Fusion-Specific Hybridization Probes

| Name | Sequence (5′-3′) |
|---|---|
| AML_KMT2A_MLLT3_2 | /5Biosg/GCTCCCCGCCCAAGTATCCCTGTAAAACAAAAACCAAAAGAAAA |
| | GTCTGAACAACCCAGTCCTGCCAGCTCCAGCTCCAGCTCCAGCTCCAA |
| | AA (SEQ ID NO: 78) |
| AML_KMT2A_MLLT10_2 | /5Biosg/GCTCCCCGCCCAAGTATCCCTGTAAAACAAAAACCAAAAGAAAA |
| | GACTTATACAAGCACTAGCAACAACTCTATATCTGGATCATTGAAGAAAA |
| | (SEQ ID NO: 79) |
| AML_PTPN11 | /5Biosg/GTGGTTTCATGGACATCTCTCTGGGAAAGAAGCAGAGAAATTAT |
| | TAACTGAAAAAGGAAAACATGGTAGTTTTCTTGTACGAGAGAGCCAAAA |
| | A (SEQ ID NO: 80) |
| AML_IDH1 | /5Biosg/CTATGATTTAGGCATAGAGAATCGTGATGCCACCAACGACCAA |
| | GTCACCAAGGATGCTGCAGAAGCTATAAAGAAGCATAATGTTGGCGTAA |
| | AA (SEQ ID NO: 81) |
| AML_TP53 | /5Biosg/TACTCCCCTGCCCTCAACAAGATGTTTTGCCAACTGGCCAAGAC |
| | CTGCCCTGTGCAGCTGTGGGTTGATTCCACACCCCCGCCCGGCACCAA |
| | AA (SEQ ID NO: 82) |

Additional embodiments utilize oligos that hybridize to constant regions artificially added by the single cell capture assay (thus “universal” towards that single cell capture platform) are used.

### (v) PCR Amplification and Detection

Particular embodiments may utilize Droplet Digital™ PCR (ddPCR™) (Bio-Rad Laboratories, Hercules, CA) for PCR amplification and detection. ddPCR technology uses a combination of microfluidics and surfactant chemistry to divide PCR samples into water-in-oil droplets. Hindson et al., Anal. Chem. 83(22): 8604-8610 (2011). The droplets support PCR amplification of the target template molecules they contain and use reagents and workflows similar to those used for most standard Taqman probe-based assays.

While ddPCR™ is one approach that can be utilized within the current disclosure, other sample partition PCR methods may also be used. These approaches are now described more generally.

Sample Partitioning. Numerous methods can be used to divide samples into discrete partitions (e.g., droplets). Exemplary partitioning systems and methods include use of one or more of emulsification, droplet actuation, microfluidics platforms, continuous-flow microfluidics, reagent immobilization, and combinations thereof. In some embodiments, partitioning is performed to divide a sample into a sufficient number of partitions such that each partition contains one or zero nucleic acid molecules. In some embodiments, the number and size of partitions is based on the concentration and volume of the bulk sample.

Methods and devices for partitioning a bulk volume into partitions by emulsification are described in Nakano et al. J. Biotechnol. 102, 117-124 (2003) and Margulies et al. Nature 437, 376-380 (2005). Systems and methods to generate “water-in-oil” droplets are described in U.S. Publication No. 2010/0173394. Microfluidics systems and methods to divide a bulk volume into partitions are described in U.S. Publication Nos. 2010/0236929; 2010/0311599; and 2010/0163412, and U.S. Pat. No. 7,851,184. Microfluidic systems and methods that generate monodisperse droplets are described in Kiss et al. Anal. Chem. 80(23), 8975-8981 (2008). Further microfluidics systems and methods for manipulating and/or partitioning samples using channels, valves, pumps, etc. are described in U.S. Pat. No. 7,842,248. Continuous-flow microfluidics systems and methods are described in Kopp et al., Science, 280, 1046-1048 (1998).

Partitioning methods can be augmented with droplet manipulation techniques, including electrical (e.g., electrostatic actuation, dielectrophoresis), magnetic, thermal (e.g., thermal Marangoni effects, thermocapillary), mechanical (e.g., surface acoustic waves, micropumping, peristaltic), optical (e.g., opto-electrowetting, optical tweezers), and chemical means (e.g., chemical gradients). In some embodiments, a droplet microactuator is supplemented with a microfluidics platform (e.g. continuous flow components).

Some embodiments use a droplet microactuator. A droplet microactuator can be capable of effecting droplet manipulation and/or operations, such as dispensing, splitting, transporting, merging, mixing, agitating, and the like. Droplet operation structures and manipulation techniques are described in U.S. Publication Nos. 2006/0194331 and 2006/0254933 and U.S. Pat. Nos. 6,911,132; 6,773,566; and 6,565,727.

The partitioned nucleic acids of a sample can be amplified by any suitable PCR methodology. Exemplary PCR types include allele-specific PCR, assembly PCR, asymmetric PCR, endpoint PCR, hot-start PCR, in situ PCR, intersequence-specific PCR, inverse PCR, linear after exponential PCR, ligation-mediated PCR, methylation-specific PCR, miniprimer PCR, multiplex ligation-dependent probe amplification, multiplex PCR, nested PCR, overlap-extension PCR, polymerase cycling assembly, qualitative PCR, quantitative PCR, real-time PCR, single-cell PCR, solid-phase PCR, thermal asymmetric interlaced PCR, touchdown PCR, universal fast walking PCR, etc. Ligase chain reaction (LCR) may also be used.

PCR may be performed with a thermostable polymerase, such as KAPA HiFi DNA Polymerase (Roche Diagnostics) Taq DNA polymerase (e.g., wild-type enzyme, a Stoffel fragment, FastStart polymerase, etc.), Pfu DNA polymerase, S-Tbr polymerase, Tth polymerase, Vent polymerase, or a combination thereof, among others.

PCR are driven by thermal cycling. Alternative amplification reactions, which may be performed isothermally, can also be used. Exemplary isothermal techniques include branched-probe DNA assays, cascade-RCA, helicase-dependent amplification, loop-mediated isothermal amplification (LAMP), nucleic acid based amplification (NASBA), nicking enzyme amplification reaction (NEAR), PAN-AC, Q-beta replicase amplification, rolling circle replication (RCA), self-sustaining sequence replication, strand-displacement amplification, etc.

Amplification may be performed with any suitable reagents (e.g. template nucleic acid (e.g. DNA or RNA)), primers, probes, buffers, replication catalyzing enzymes (e.g. DNA polymerase, RNA polymerase), nucleotides, salts (e.g. MgCl₂), etc. In some embodiments, an amplification mixture includes any combination of at least one primer or primer pair, at least one probe, at least one replication enzyme (e.g., at least one polymerase), and deoxynucleotide (and/or nucleotide) triphosphates (dNTPs and/or NTPs), etc.

Amplification reagents can be added to a sample prior to partitioning, concurrently with partitioning and/or after partitioning has occurred. In some embodiments, all partitions are subjected to amplification conditions (e.g. reagents and thermal cycling), but amplification only occurs in partitions containing target nucleic acids (e.g. nucleic acids containing sequences complementary to primers added to the sample). The template nucleic acid can be the limiting reagent in a partitioned amplification reaction. In some embodiments, a partition contains one or zero target (e.g. template) nucleic acid molecules.

As indicated previously, in some embodiments, nucleic acid targets, primers, and/or probes are immobilized to a surface, for example, a substrate, plate, array, bead, particle, etc. Immobilization of one or more reagents provides (or assists in) one or more of: partitioning of reagents (e.g. target nucleic acids, primers, probes, etc.), controlling the number of reagents per partition, and/or controlling the ratio of one reagent to another in each partition. In some embodiments, assay reagents and/or target nucleic acids are immobilized to a surface while retaining the capability to interact and/or react with other reagents (e.g. reagent dispensed from a microfluidic platform, a droplet microactuator, etc.). In some embodiments, reagents are immobilized on a substrate and droplets or partitioned reagents are brought into contact with the immobilized reagents. Techniques for immobilization of nucleic acids and other reagents to surfaces are well understood by those of ordinary in the art. See, for example, U.S. Pat. No. 5,472,881 and Taira et al. Biotechnol. Bioeng. 89(7), 835-8 (2005).

Detection methods can be utilized to identify sample partitions containing amplified target(s). Detection can be based on one or more characteristics of a sample partition such as a physical, chemical, luminescent, or electrical aspects, which correlate with amplification.

In particular embodiments, fluorescence detection methods are used to detect amplified target(s), and/or identification of sample partitions containing amplified target(s). Exemplary fluorescent detection reagents include TaqMan probes, SYBR Green fluorescent probes, molecular beacon probes, scorpion probes, and/or LightUp Probes® (LightUp Technologies AB, Huddinge, Sweden). Additional detection reagents and methods are described in, for example, U.S. Pat. Nos. 5,945,283; 5,210,015; 5,538,848; and 5,863,736; PCT Publication WO 97/22719; and publications: Gibson et al., Genome Research, 6, 995-1001 (1996); Heid et al., Genome Research, 6, 986-994 (1996); Holland et al., Proc. Natl. Acad. Sci. USA 88, 7276-7280, (1991); Livak et al., Genome Research, 4, 357-362 (1995); Piatek et al., Nat. Biotechnol. 16, 359-63 (1998); Neri et al., Advances in Nucleic Acid and Protein Analysis, 3826, 117-125 (2000); Compton, Nature 350, 91-92 (1991); Thelwell et al., Nucleic Acids Research, 28, 3752-3761 (2000); Tyagi and Kramer, Nat. Biotechnol. 14, 303-308 (1996); Tyagi et al., Nat. Biotechnol. 16, 49-53 (1998); and Sohn et al., Proc. Natl. Acad. Sci. U.S.A. 97, 10687-10690 (2000).

In some embodiments, detection reagents are included with amplification reagents added to the bulk or partitioned sample. In some embodiments, amplification reagents also serve as detection reagents. In some embodiments, detection reagents are added to partitions following amplification. In some embodiments, measurements of the absolute copy number and the relative proportion of target nucleic acids in a sample (e.g. relative to other targets nucleic acids, relative to non-target nucleic acids, relative to total nucleic acids, etc.) can be measured based on the detection of sample partitions containing amplified targets.

In some embodiments, following amplification, sample partitions containing amplified target(s) are sorted from sample partitions not containing amplified targets or from sample partitions containing other amplified target(s). In some embodiments, sample partitions are sorted following amplification based on physical, chemical, and/or optical characteristics of the sample partition, the nucleic acids therein (e.g. concentration), and/or status of detection reagents. In some embodiments, individual sample partitions are isolated for subsequent manipulation, processing, and/or analysis of the amplified target(s) therein. In some embodiments, sample partitions containing similar characteristics (e.g. same fluorescent labels, similar nucleic acid concentrations, etc.) are grouped (e.g. into packets) for subsequent manipulation, processing, and/or analysis.

Particular embodiments utilize Next Generation Sequencing (NGS) or Third Generation Sequencing (TGS). In particular embodiments, sequencing with commercially available NGS or TGS platforms may be conducted with the following steps. First, DNA sequencing libraries may be generated by clonal amplification by PCR in vitro. Second, the DNA may be sequenced by synthesis, such that the DNA sequence is determined by the addition of nucleotides to the complementary strand rather through chain-termination chemistry. Third, the spatially segregated, amplified DNA templates may be sequenced simultaneously in a massively parallel fashion without the requirement for a physical separation step. While these steps are followed in most NGS and TGS platforms, each utilizes a different strategy (see e.g., Anderson, M. W. and Schrijver, I., 2010, Genes, 1: 38-69.). Examples of NGS and TGS platforms include Oxford Nanopore Technologies, Roche 454, GS FLX Titanium, Illumina, HiSeq 2000, Genome Analyzer IX, IIE, IScanSQ, Life Technologies Solid 4, Helicos Biosciences Heliscope, Pacific Biosciences (PacBio) SMART and PacBio HiFi.

In particular embodiments, DNA segments can undergo an amplification as part of sequencing. In embodiments where an amplification process was used to create a target-increased sample, this amplification would be a second amplification step. The second amplification can provide a stronger signal than if the second amplification was not performed.

In particular embodiments, the methods include detecting a control. A control can refer to an RNA or DNA sequence that is “spiked” into a sample at a known or otherwise specified amount. In particular embodiments, the control is spiked into the sample at a known quantity (e.g., known copy number), which can be useful, for example, to determine the absolute quantity of an RNA or DNA sequence (e.g., a unique sequence).

Embodiments disclosed herein can be used with high throughput screening (HTS). Typically, HTS refers to a format that performs at least 100 assays, at least 500 assays, at least 1000 assays, at least 5000 assays, at least 10,000 assays, or more per day. When enumerating assays, either the number of samples or the number of markers assayed can be considered.

Generally, HTS methods involve a logical or physical array of either samples, or the nucleic acid or protein markers, or both. Appropriate array formats include both liquid and solid phase arrays. For example, assays employing liquid phase arrays, e.g., for hybridization of nucleic acids, binding of antibodies or other receptors to ligand, etc., can be performed in multiwell or microtiter plates. Microtiter plates with 96, 384, or 1536 wells are widely available, and even higher numbers of wells, e.g., 3456 and 9600 can be used. In general, the choice of microtiter plates is determined by the methods and equipment, e.g., robotic handling and loading systems, used for sample preparation and analysis.

HTS assays and screening systems are commercially available from, for example, Zymark Corp. (Hopkinton, MA); Air Technical Industries (Mentor, OH); Beckman Instruments, Inc. (Fullerton, CA); Precision Systems, Inc. (Natick, MA), etc. These systems typically automate entire procedures including all sample and reagent pipetting, liquid dispensing, timed incubations, and final readings of the microplate in detector(s) appropriate for the assay. These configurable systems provide HTS as well as a high degree of flexibility and customization. The manufacturers of such systems provide detailed protocols for the various methods of HTS.

### (vi) Reference Levels & Analysis

As indicated previously, down regulation or loss of HLA expression can be assessed and the HLA expression level can be compared to a relevant reference level. The value can be one or more numerical values resulting from the assaying of a sample, and can be derived, e.g., by measuring HLA expression in the sample by an assay, or from a dataset obtained from a provider such as a laboratory, or from a dataset stored on a server.

In particular embodiments, a gene with reduced expression by a cancer cell includes a gene with at least 10% decreased expression (for example, at least 15%, 20%, 25%, 30%, 40%, 50%, 60%, 70%, 80%, 90%, 95%, or even 100% decreased expression) compared to an appropriate control. In some examples, the control is expression of the same HLA gene in normal (e.g., non-cancer) cells from the same subject. In other examples, the control can be a reference value, such as an average expression level for normal (non-cancer) cells in a population. A gene with reduced transcript copy number in a cancer cell includes a gene with reduced transcript copy number compared to a control, for example, having expression from a single allele (e.g., loss of heterozygosity) or no expression at all. In some examples, a cancer cell is identified as having a reduced transcript copy number of an HLA allele if it is present in less than 90% of the cancer cell cells examined (such as less than 80%, 70%, 60%, 50%, 40%, 30%, 20%, or 10% of cancer cell cells, for example, 10-90%, 25-75%, 40-80%, or 20-50% of cancer cell cells).

In the broadest sense, expression level values may be qualitative or quantitative. As such, where detection is qualitative, systems and methods provide a reading or evaluation, e.g., assessment, of HLA expression in the sample being assayed. In further embodiments, the systems and methods provide a quantitative detection of HLA expression, i.e., an evaluation or assessment of the actual amount or relative abundance of HLA expression in the sample being assayed. In such embodiments, the quantitative detection may be absolute or relative. As such, the term “quantifying” when used in the context of quantifying HLA expression in a sample can refer to absolute or to relative quantification. Absolute quantification can be accomplished by inclusion of samples with known HLA expression as one or more control markers and referencing, e.g., normalizing, the detected HLA expression with the known control expression (e.g., through generation of a standard curve). Alternatively, relative quantification can be accomplished by comparison of generated HLA expression between to provide a relative quantification, e.g., relative to each other. The actual measurement of values for HLA expression can be determined using single cell RNA-based sequencing as disclosed herein.

As stated previously, detected HLA expression levels can be compared to one or more reference levels. Reference levels can be obtained from one or more relevant datasets. A “dataset” as used herein is a set of numerical values resulting from evaluation of a sample (or population of samples) under a desired condition. The values of the dataset can be obtained, for example, by experimentally obtaining measures from sample(s) and constructing a dataset from these measurements. As is understood by one of ordinary skill in the art, the reference level can be based on e.g., any mathematical or statistical formula useful and known in the art for arriving at a meaningful aggregate reference level from a collection of individual datapoints; e.g., mean, median, median of the mean, etc.

A reference level from a dataset can be derived from previous measures derived from a population. A “population” is any grouping of subjects or samples of like specified characteristics. The grouping could be according to, for example, clinical parameters, clinical assessments, therapeutic regimens, disease status, severity of cancer, etc. In particular embodiments, a population is a group of subjects with leukemia at initial diagnosis or relapse.

In particular embodiments, conclusions are drawn based on whether an HLA expression level is statistically significantly different or not statistically significantly different from a reference level. A measure is not statistically significantly different if the difference is within a level that would be expected to occur based on chance alone. In contrast, a statistically significant difference is one that is greater than what would be expected to occur by chance alone. Statistical significance or lack thereof can be determined by any of various methods well-known in the art. An example of a commonly used measure of statistical significance is the p-value. The p-value represents the probability of obtaining a given result equivalent to a particular datapoint, where the datapoint is the result of random chance alone. A result is often considered significant (not random chance) at a p-value less than 0.05.

In particular embodiments, HLA expression levels can be subjected to an analytic process with chosen parameters. The parameters of the analytic process may be those disclosed herein or those derived using the guidelines described herein. The analytic process used to generate a result may be any type of process capable of distinguishing HLA expression levels or variant expression, for example, a linear algorithm, a quadratic algorithm, a decision tree algorithm, or a voting algorithm. The analytic process may set a threshold for determining the probability that a sample belongs to a given class. The probability preferably is at least 60%, at least 70%, at least 80%, at least 90%, at least 95% or higher.

Particular embodiments disclosed herein utilize R. R is machine learning and statistical computing software. There are R packages developed for specific functions, such as to annotate transcripts (ex. TransAT). Transcript annotations can be visualized on cells to identify clusters of similar cells using methods such as UMAP plots (a non-linear dimensionality reduction technique used to visualize the similarity of cell transcriptomes (github.com/lmcinnes/umap)), t-distributed stochastic neighbor embedding (t-SNE), and global t-distributed stochastic neighbor embedding (g-SNE).

### (vii) Kits

Kits disclosed herein include materials to assay a sample for HLA isoform variations and/or expression levels. In particular embodiments, the kits at least a subset of probes disclosed in Table 1, or functional variants thereof. Particular embodiments include all probes disclosed in Table 1, or functional variants thereof. Particular embodiments include an RNA polymerase to generate cDNA. Additional examples can include materials to conduct PCR, such as at least one primer or primer pair, at least one probe, at least one replication enzyme (e.g., at least one polymerase), and deoxynucleotide (and/or nucleotide) triphosphates (dNTPs and/or NTPs), etc. Certain examples include the blocking oligos disclosed in Table 2 or functional variants thereof.

Additional embodiments include detection reagents within kits. Exemplary detection reagents can include radioactive isotopes or radiolabels (e.g., 32P and 13C), enzymes (e.g., luciferase, HRP and AP), dyes (e.g., rhodamine and cyanine), fluorescent tags or dyes (e.g., GFP, YFP, FITC), magnetic beads, or biotin. In particular embodiments, the detectable label is fluorescein, GFP, rhodamine, cyanine dyes, Alexa dyes, luciferase, or a radiolabels. TaqMan probes, SYBR Green fluorescent probes, molecular beacon probes, scorpion probes, and/or LightUp Probes® (LightUp Technologies) may also be used.

Particular embodiments can include reference levels and/or control conditions (positive and/or negative) to guide a user in interpreting results.

Instructions for carrying out and interpreting HLAisoform variations and expression levels, including, optionally, instructions for generating a score, can also be included in a kit. Instructions can be provided in written, taped, videoed, VCR, CD-ROM, flashdrive, USB formats or can be provided on a website or other remote location.

### (viii) Exemplary Embodiments

1. A panel of hybridization probes or functional variants thereof that hybridize to all known HLA alleles in the HLA regions HLA-A, -B, -C, -E, -F, -G, -H, -J, -K, -L, -T, -V, -W, -Y, -DMA, -DMB, -DOA, -DOB, -DPA1, -DPA2, -DPB1, -DPB2, -DQA1, -DQA2, -DQB1, -DRA, -DRB1, -DRB2, -DRB3, -DRB4, -DRB5, -DRB6, -DRB7, -DRB8, -DRB9, HFE, MICA, MICB, TAP1, and TAP.
2. A panel of hybridization probes or functional variants thereof that hybridize to a subset of all known HLA alleles in the HLA regions HLA-A, -B, -C, -E, -F, -G, -H, -J, -K, -L, -T, -V, -W, -Y, -DMA, -DMB, -DOA, -DOB, -DPA1, -DPA2, -DPB1, -DPB2, -DQA1, -DQA2, -DQB1, -DRA, -DRB1, -DRB2, -DRB3, -DRB4, -DRB5, -DRB6, -DRB7, -DRB8, -DRB9, HFE, MICA, MICB, TAP1, and TAP.
3. The panel of embodiment 2, wherein the subset hybridizes to Class I HLA alleles.
4. The panel of embodiment 3, wherein the subset hybridizes to HLA-A, HLA-B, HLA-C.
5. The panel of embodiment 4, wherein the hybridization probes include PanCls1_a, PanCls1_b, PanCls1_c, and PanCls1_d.
6. The panel of embodiment 3, wherein the subset hybridizes to HLA-E, HLA-F, HLA-G, MICA, MICB. and HFE.
7. The panel of embodiment 6, wherein the hybridization probes include PanCls1_a, PanCls1_b, PanCls1_c, and PanCls1_d, MICAB_a, MICAB_b, HFE_a, and HFE_b.
8. The panel of embodiment 3, wherein the subset hybridizes to HLA-A, HLA-B, HLA-C, HLA-E, HLA-F, HLA-G, MICA, MICB, and HFE.
9. The panel of embodiment 8, wherein the hybridization probes include PanCls1_a, PanCls1_b, PanCls1_c, and PanCls1_d, MICAB_a, MICAB_b, HFE_a, and HFE_b.
10. The subset of any of embodiments 2-9, wherein the subset hybridizes to MHC class II molecules.
11. The panel of embodiment 10, wherein the subset hybridizes to HLA-DR, HLA-DQ, and HLA-DP,
12. The panel of embodiment 11, wherein the hybridization probes include DRA_a, DRA_b, DRB_-a, DRB_b, DRB_c, DQA_a, DQA_b, DQB/DPB_a, DQB/DPB_b, DQB/DPB_c, and DQB/DPB_d.
13. The panel of embodiment 10, wherein the subset hybridizes to HLA-DM and HLA-DO.
14. The panel of embodiment 13, wherein the hybridization probes include DMA_a, DMA_b, DMB_a, DM3_b, DOA_a, DOA_b, DOB_a, and DOB_b
15. The panel of embodiment 10, wherein the subset hybridizes to HLA-DR, HLA-DQ, HLA-DP, HLA-DM and HLA-DO.
16. The panel of embodiment 15, wherein the hybridization probes include DRA_a, DRA_b, DRB_a, DRB_b, DRB_c, DQA_a, DQA_b, DQB/DPB_a, DQB/DPB_b, DQB/DPB_c, DQB/DPB_d, DMA_a, DMA_b, DMB_a, DMB_b, DOA_a, DOA_b, DOB_a, and DOB_b.
17. The panel of embodiment 2, wherein the subset hybridizes to HLA-A, HLA-B, HLA-C, HLA-DR, HLA-DQ, and HLA-DP.
18. The panel of embodiment 17, wherein the hybridization probes include PanCls1_a, PanCls1_b, PanCls1_c, PanCls1_d, DRA_a, DRA_b, DRB_a, DRB_b, DRB_c, DQA_a, DQA_b, DQB/DPB_a, DQB/DPB_b, DQB/DPB_c, and DQB/DPB_d.
19. The panel of an of embodiments 1-18 including a sequence:

(SEQ ID NO: 1)
ACCAGGCCTGCAGGGGATGGAACCTTCCAGAAGTGGGCGKCTGTGGTGGTRCCTTCTGGASAGGAGCAGAGAT
ACACCTGCCATGTGCAGCATGAGG;
(SEQ ID NO: 2)
GTYTSCCCAAGCCCCTCACCCTGAGATGGGAGCYGTCTTCCCAGCCCACCATCCCCATCGTGGGCATCATTGCT
GGCCTGGTTCTCYTTGGAGCTGTGW;
(SEQ ID NO: 3)
GGCAGCTGTGGTGGTGCCTTCTGGAGAAGAGCAGAGATACACATGCCATGTACAGCATGAGGGGCTGCCRAAG
CCCCTCACCCTGAGATGGG;
(SEQ ID NO: 4)
ACYTGGGGTCCGACGGWCGCCTCMTCCGCGGGTATGAACAGTATGCCTACGATGGCAAGGATTACCTCGCCC
TGAACGAGGACCTGCGCTCCT;
(SEQ ID NO: 5)
GCGAGTGGATGATCCAGCAAATAGGGCCAAAACTTGATGGGAAAATCCCGGTGTCCAGAGGGTTTCCTATCGCT
GAAGTGTTCACGCTGAAGCCCCTG;
(SEQ ID NO: 6)
GAGTTTGGCAAGCCCAACACTTTGGTCTGTTTTGTCAGTAATCTCTTCCCACCCATGCTGACAGTGAACTGGCAG
CATCATTCCRTCCCTGTGGAAGG;
(SEQ ID NO: 7)
CCCCTCTTACGGGGACACTTACACCTGTGTGGTAGAGCACAYTGGGGCTCCTGAGCCCATCCTTCGGGACTGG
ACACCTGGGCTGTCCC;
(SEQ ID NO: 8)
CCATGCAGACCCTGAAGGTTTCTGTGTCTGCAGTGACTCTGGGCCTGGGCCTCATCATCTTCTCTCTTGGTGTG
ATCAGCTGGCGGAGAGCTGGCC;
(SEQ ID NO: 9)
GGGCCAGCCCAACATCCTCATCTGCATCGTGGACAACATCTTCCCCCCTGTGATCAATATCACCTGGCTRCGCA
ACGGCCAAACTGTCACTGAGGGAG;
(SEQ ID NO: 10)
TGGCCCAGACCAGCTTCTATTCCCAGCCTGACCATTTGTTCCGCAAGTTCCACTACCTGCCCTTCGTGCCCTCA
GCCGAGGACGTCTATGACTGCCA;
(SEQ ID NO: 11)
GGGATGTTTGTGGCATTGACCAAGCTGGGGCAGCCAGATGCTGAGCAGTGGAACAGCCGGCTGGATCTCTTGG
ARAGGAGCAGACAGGCCGTGG;
(SEQ ID NO: 12)
ATGGGGTCTGTAGACACAACTACAGGCTGGGCGCACCCTTCACTGTGGGGAGAAAAGTGCAACCAGAGGTGAC
AGTGTACCCAGAGAGGACCCC;
(SEQ ID NO: 13)
CCAGTGCTCAACGTCACRTGGCTGTGCAAYGGGGAGCYGGTCACTGAGGGTGTCGCTGAGAGCCTCTTCCTGC
CCAGAACAGATTACAGCT;
(SEQ ID NO: 14)
TCCACAAGTTCCATTACCTGACCTTTGTGCCCTCAGCAGAGGACKTCTATGACTGCAGGGTGGAGCACTGGGGC
TTGGACCAGCCGCTCCT;
(SEQ ID NO: 15)
AGGTGGAGCACTGGGGCCTGGAYSAGCCTCTTCTGAAACACTGGGAGCCTGAGATTCCAGCMCCTATGTCAGA
GCTCACAGAGACTKTGGTCTGY;
(SEQ ID NO: 16)
RCCCTGGGRTTGTCTGTGGGCCTCRTGGGCATTGTGGTGGGCACTGTCTTCATCATCCRAGGCCTGCGTTCAG
TTGGTGCTTCCAGACACCARGGG;
(SEQ ID NO: 17)
GGGCCCYTGCAGCACCACAACCTGCTTGTCTGCCACGTGACRGATTTCTACCCAGGCAGCATTCAAGTCCGATG
GTTCCTGAATGGACAGG;
(SEQ ID NO: 18)
AGGAAACAGCTGGGGTCGTGTCCACCAACCTGATCCGTAATGGAGACTGGACCTTCCAGATCCTGGTGATGCT
GGAAATGACCCCCCAGCA;
(SEQ ID NO: 19)
TCCCCATCCAGGACAGAGGCCCTCAACCACCACAACCTGCTGRTCTGCTCRGTGACAGATTTCTATCCAGSCCA
GATCAAAGTCCGGTGGTTTCGG;
(SEQ ID NO: 20)
AATGRYCAGGAGGAGACARCYGGCGTTGTGTCCACCCCCCTTATTAGGAAYGGTGACTGGACCTTCCAGATCCT
GGTGATGCTGGAAATGACTCCCCA;
(SEQ ID NO: 21)
GGGAAGACCACCTTTTCCGCAAGTTCCACTATCTCCCCTTCCTGCCCTCAACTGAGGACGTTTACGACTGCAGG
GTGGAGCACTGGGGCTTGG;
(SEQ ID NO: 22)
ATGAGCCTCTTCTCAAGCACTGGGAGTTTGATGCTCCAAGCCCTCTCCCAGAGACTACAGAGAAYGTGGTGTGT
GCCCTGGGCCTGACTGTGGG;
(SEQ ID NO: 23)
TKCAAAGACCCAGCCCCTGCAGCACCACAACCTCCTGGTCTGYTCTGTGARTGGTTTCTATCCAGGCAGCATTG
AAGTCAGGTGGTTCCKGAAY;
(SEQ ID NO: 24)
RGCCAGGAAGAGAAGRCTGGGGTGGTGTCCACRGGCCTGATCCASAATGGAGACTGGACCTTCCAGACCCTG
GTGATGCTGGAAACAGTTCCTCGG;
(SEQ ID NO: 25)
GCGGAGCTGGAGCGGAGAAGAGTCCAGGAATRCAAACAGCCAGAAGAACCTCTTGGGCTGCTTGCGGGGTCT
GTTGGACACCTACTGCAGACA;
(SEQ ID NO: 26)
GCCCTGACACACTGGATTGGAGAGCAGCAGAACCCAGGGCCTGGCCCACCAAGCTGGAGTGGGAAAGGCACA
AGATTCGGGCCAGGCAGAACAGG;
(SEQ ID NO: 27)
GCCTACCTGGAGAGGGACTGCCCTGCACAGCTGCAGCAGTTGCTGGAGCTGGGGAGAGGTGTTTTGGACCAA
CAAGTGCCTCCTTTGGTGAAGGTGA;
(SEQ ID NO: 28)
AGTCAGGGTTTCTYRCTGAGGTACATCTGGATGGTCAGCCCTTCCTGCGCTRTGACAGGCAGAAAYGCAGGGC;
(SEQ ID NO: 29)
AAAGCCCCAGGGACAGTGGGCAGAAGATGTCCTGGGAAATAAGACATGGGACAGAGAGACCAGRGACTTGACA
GGGA;
(SEQ ID NO: 30)
TGGGGATCAGTGTCCCTCACCATGGTCACCCTGRTCACCCTGCCTCTGCTTTTCCTTCTGCCCAAGAAGGTGGG
AAAATGGTACCAGTTGCTGG;
(SEQ ID NO: 31)
AAGTGCAGGTGCGGGAATCTCTGGCAAAGTCCAGCCAGGTGGCCATTGAGGYYCTGTCGGCCATGCCTACAGT
TCGAAGCTTTGCCAACGAGGAGG;
(SEQ ID NO: 32)
GCCGTTGGAGGGCTGCAGACCGTTCGCAGTTTTGGGGCCGAGGAGCATGAAGTCTGTCGCTATAAAGAGGCCC
TTGAACAATGTCGGCAGC;
or
(SEQ ID NO: 33)
TGTATTGGCGGAGAGACCTGGAACGCGCCTTGTACCTGCTCRTAAGGAGGGTGCTGCACTTGGGKGTGCAGAT
GCTGATGCTGAGCTGTGG

wherein R=A/G, Y=C/T, M=A/C, K=G/T, S=C/G, W=A/T, B=C/G/T, D=AG/T, H=A/C/T, V=A/C/G, andN=NC/G/T
   or a sequence having at least 60%, 65%, 70%, 75%, 80%, 85%, 90%, 95%, 96%, 97%, 98%, or 99% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, or SEQ ID NO: 33.
20. The panel of any of embodiments 1-19, including a sequence having 95%, 96%, 97%, 98%, or 99% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, or SEQ ID NO: 33.
21. The panel of any of embodiments 1-20, including a sequence having the sequence of SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, or SEQ ID NO: 69 wherein R=A/G, Y=C/T, M=A/C, K=G/T, S=C/G, W=NT, B=C/G/T, D=AG/T, H=A/C/T, V=A/C/G, and N=A/C/G/T
   or a sequence having at least 60%, 65%, 70%, 75%, 80%, 85%, 90%, 95%, 96%, 97%, 98%, or 99% sequence identity to SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, or SEQ ID NO: 69.
22. The panel of any of embodiments 1-21, having 95%, 96%, 97%, 98%, or 99% sequence identity to SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, or SEQ ID NO: 69.
23. The panel of hybridization probes of any of embodiments 1-22, wherein the probes are linked to a detectable label,
24. The panel of hybridization probes of any of embodiments 1-23, wherein the probes are biotinylated.
25. The panel of hybridization probes of any of embodiments 1-24, wherein the probes are linked to a detectable label at the 5 end of each probe,
26. Use of a panel of hybridization probes of any of embodiments 1-25 to capture cDNA within a cDNA pool.
27. The use of embodiment 26, wherein the cDNA pool is an amplified cDNA pool.
28. A method including polymerase chain reaction (PCR) amplifying the DNA captured according to the use of embodiment 26.
29. The method of embodiment 28, wherein the captured cDNA includes a barcode and a unique molecular identifier.
30. The method of embodiment 28 or 29, wherein the PCR amplifying is in the presence of blocking oligos.
31. The method of embodiment 30, wherein the blocking oligos include a sequence: CTACACGACGCTCTTCCGATCTIIIIIIIIIIIIIIIIIIIIIIIIIITTTCTTATATGGG (SEQ ID NO: 34); AAAAAAAAAAAAAAAAAAAAGTACTCTGCGTTGATACCACTGCTT (SEQ ID NO: 35); AAGCAGTGGTATCAACGCAGAGTACATGGG/3SpC3/(SEQ ID NO: 70); or AAGCAGTGGTATCAACGCAGAGTACATGGGAAAAAAAAAIAAAAAAAAAAIIIIIIIIIIIIIIIIIIIIIIIIIIIIAGATCGGAAGAGC GTCGTGTAG (SEQ ID NO: 36) or a sequence having at least 60%, 65%, 70%, 75%, 80%, 85%, 90%, 95%, 96%, 97%, 98%, or 99% sequence identity to SEQ ID NO: 34, SEQ ID NO: 35, or SEQ ID NO: 36.
32. The method of embodiment 30, wherein the blocking oligos include a sequence having 95%, 96%, 97%, 98%, or 99% sequence identity to SEQ ID NO: 34, SEQ ID NO: 35, or SEQ ID NO: 70, SEQ ID NO: 36.
33. A method including sequencing the amplified DNA any of embodiments 30-32.
34. The method of embodiment 33, further including utilizing the sequencing to identify an HLA allele expressed by a cell.
35. The method of embodiment 34. wherein the cell is a cancer cell,
36. The method of embodiment 35, cancer cell is a leukeria cell.
37. The method of embodiment 36, wherein the leukemia cell is an acute myeloid leukemia (AML) cell, acute lymphoblastic leukemia (ALL) cell, chronic myelogenous leukemia (CML) cell, chronic myelomonocytic leukemia (CML) cell, mast cell leukemia cell, myelodysplastic syndrome (MDS) cell, B-cell acute lymphoblastic leukemia (B-ALL) cell, T-cell acute lymphoblastic leukemia (T-ALL) cell, or megakaryocytic leukeria cell.
38. The method of any of embodiments 34-37, wherein the cell is obtained from a human cancer patient.
39. The method of any of embodiments 34-38, wherein the cell is obtained from a human cancer patient at the time of diagnosis.
40. The method of any of embodiments 34-39, wherein the cell is obtained from a human cancer patient at the time of relapse.
41. The method of any of embodiments 34-40, wherein the cell is obtained from a human cancer patient after a hematopoietic cell transplant,
42. The method of any of embodiments 34-41, wherein the cell is obtained from a bone marrow aspirate sample.
43. A method including determining the copy number of HLA alleles from a cell based on the amplifying, bar code, and unique molecular identifier of embodiment 29.
44. A kit including (i) a panel of hybridization probes or functional variants thereof that hybridize to all known HLA alleles in the HLA regions HLA-A, -B, -C, -E, -F, -G, -H, -J, -K, -L, -T, -V, -W, -Y, -DMA, -DMB, -DOA, -DOB, -DPA1, -DPA2, -DPB1, -DPB2, -DQA1, -DQA2, -DQB1, -DRA, -DRB1, -DRB2, -DRB3, -DRB4, -DRB5, -DRB6, -DRB7, -DRB8, -DRB9, HFE, MICA, MICB, TAP1, and TAP2; and (ii) a set of blocking oligos.
45. A kit including (i) a panel of hybridization probes or functional variants thereof that hybridize to a subset of all known HLA alleles in the HLA regions HLA-A, -B, -C, -E, -F, -G, -H, -J, -K, -L, -T, -V, -W, -Y, -DMA, -DMB, -DOA, -DOB, -DPA1, -DPA2, -DPB1, -DPB2, -DQA1, -DQA2, -DQB1, -DRA, -DRB1, -DRB2, -DRB3, -DRB4, -DRB5, -DRB6, -DRB7, -DRB8, -DRB9, HFE, MICA, MICB, TAP1, and TAP2; and (ii) a set of blocking oligos.
46. The kit of embodiment 44, wherein the subset hybridizes to Class HLA alleles.
47. The kit of embodiment 45, wherein the subset hybridizes to HLA-A, HLA-B, -1LA-C,
48. The kit of embodiment 46, wherein the hybridization probes include PanCls1l_a, PanCls1_b, PanCls1_c, and PanCls1_d.
49. The kit of embodiment 45, wherein the subset hybridizes to HLA-E, HLA-F, HLA-G, MICA, MICB and HFE,
50. The kit of embodiment 47, wherein the hybridization probes include PanCls1_a, PanCls1_b, PanCls1_c, and PanCls1_d, MICAB_a, MICAB_b, HFE_a, and HFE_b.
51. The kit of embodiment 45, wherein the subset hybridizes to HLA-A, HLA-B, HLA-C, HLA-E, HLA-F, HLA-G, MICA, MICB, and HFE.
52. The kit of embodiment 50, wherein the hybridization probes include PanCls1_a, PanCls1_b, PanCls1_c, and PanCls1_d, MICAB_a, MICAB_b, HFE_a, and HFE_b,
53. The kit of embodiment 44, wherein the subset hybridizes to MHC class II molecules.
54. The kit of embodiment 52, wherein the subset hybridizes to HLA-DR, HLA-DQ, and HLA-DP.
55. The kit of embodiment 53, wherein the hybridization probes include DRA_a, DRA_b, DRB_a, DRB_b, DRB_c, DQA_a, DQA_b, DQB/DPB_a, DQB/DPB_b, DQB/DPB_c, and DQB/DPB_d.
56. The kit of embodiment 52, wherein the subset hybridizes to HLA-DM and HLA-DO.
57. The kit of embodiment 55, wherein the hybridization probes include DMA_a, DMA_b, DMB_a, DMB_b, DOA_a, DOA_b, DOB_a, and DOB_b
58. The kit of embodiment 52, wherein the subset hybridizes to HLA-DR, HLA-DQ, HLA-DP, HLA-DM and HLA-DO.
59. The kit of embodiment 57, wherein the hybridization probes include DRA_a, DRA_b, DRB_a, DRB_b, DRB_c, DQA_a, DQA_b, DQB/DPB_a, DQB/DPB_b, DQB/DPB_c, DQB/DPB_d, DMA_a, DMA_b, DMB_a, DMB_b, DOA_a, DOA_b, DOB_a, and DOB_b.
60. The kit of embodiment 44, wherein the subset hybridizes to HLA-A, HLA-B, HLA-C, HLA-DR, HLA-DQ, and HLA-DP.
61. The kit of embodiment 59, wherein the hybridization probes include PanCls1_a, PanCls1_b, PanCls1_c, PanCls1_d, DRA_a, DRA_b, DRB_a, DRB_b, DRB_c, DQA_a, DQA_b, DQB/DPB_a, DQB/DPB_b, DQB/DPB_c, and DQB/DPB_d.
62. The kit of any of embodiments 44-61, including a sequence:

(SEQ ID NO: 1)
ACCAGGCCTGCAGGGGATGGAACCTTCCAGAAGTGGGCGKCTGTGGTGGTRCCTTCTGGASAGGAGCAGAGAT
ACACCTGCCATGTGCAGCATGAGG;
(SEQ ID NO: 2)
GTYTSCCCAAGCCCCTCACCCTGAGATGGGAGCYGTCTTCCCAGCCCACCATCCCCATCGTGGGCATCATTGCT
GGCCTGGTTCTCYTTGGAGCTGTGW;
(SEQ ID NO: 3)
GGCAGCTGTGGTGGTGCCTTCTGGAGAAGAGCAGAGATACACATGCCATGTACAGCATGAGGGGCTGCCRAAG
CCCCTCACCCTGAGATGGG;
(SEQ ID NO: 4)
ACYTGGGGTCCGACGGWCGCCTCMTCCGCGGGTATGAACAGTATGCCTACGATGGCAAGGATTACCTCGCCC
TGAACGAGGACCTGCGCTCCT;
(SEQ ID NO: 5)
GCGAGTGGATGATCCAGCAAATAGGGCCAAAACTTGATGGGAAAATCCCGGTGTCCAGAGGGTTTCCTATCGCT
GAAGTGTTCACGCTGAAGCCCCTG;
(SEQ ID NO: 6)
GAGTTTGGCAAGCCCAACACTTTGGTCTGTTTTGTCAGTAATCTCTTCCCACCCATGCTGACAGTGAACTGGCAG
CATCATTCCRTCCCTGTGGAAGG;
(SEQ ID NO: 7)
CCCCTCTTACGGGGACACTTACACCTGTGTGGTAGAGCACAYTGGGGCTCCTGAGCCCATCCTTCGGGACTGG
ACACCTGGGCTGTCCC;
(SEQ ID NO: 8)
CCATGCAGACCCTGAAGGTTTCTGTGTCTGCAGTGACTCTGGGCCTGGGCCTCATCATCTTCTCTCTTGGTGTG
ATCAGCTGGCGGAGAGCTGGCC;
(SEQ ID NO: 9)
GGGCCAGCCCAACATCCTCATCTGCATCGTGGACAACATCTTCCCCCCTGTGATCAATATCACCTGGCTRCGCA
ACGGCCAAACTGTCACTGAGGGAG;
(SEQ ID NO: 10)
TGGCCCAGACCAGCTTCTATTCCCAGCCTGACCATTTGTTCCGCAAGTTCCACTACCTGCCCTTCGTGCCCTCA
GCCGAGGACGTCTATGACTGCCA;
(SEQ ID NO: 11)
GGGATGTTTGTGGCATTGACCAAGCTGGGGCAGCCAGATGCTGAGCAGTGGAACAGCCGGCTGGATCTCTTGG
ARAGGAGCAGACAGGCCGTGG;
(SEQ ID NO: 12)
ATGGGGTCTGTAGACACAACTACAGGCTGGGCGCACCCTTCACTGTGGGGAGAAAAGTGCAACCAGAGGTGAC
AGTGTACCCAGAGAGGACCCC;
(SEQ ID NO: 13)
CCAGTGCTCAACGTCACRTGGCTGTGCAAYGGGGAGCYGGTCACTGAGGGTGTCGCTGAGAGCCTCTTCCTGC
CCAGAACAGATTACAGCT;
(SEQ ID NO: 14)
TCCACAAGTTCCATTACCTGACCTTTGTGCCCTCAGCAGAGGACKTCTATGACTGCAGGGTGGAGCACTGGGGC
TTGGACCAGCCGCTCCT;
(SEQ ID NO: 15)
AGGTGGAGCACTGGGGCCTGGAYSAGCCTCTTCTGAAACACTGGGAGCCTGAGATTCCAGCMCCTATGTCAGA
GCTCACAGAGACTKTGGTCTGY;
(SEQ ID NO: 16)
RCCCTGGGRTTGTCTGTGGGCCTCRTGGGCATTGTGGTGGGCACTGTCTTCATCATCCRAGGCCTGCGTTCAG
TTGGTGCTTCCAGACACCARGGG;
(SEQ ID NO: 17)
GGGCCCYTGCAGCACCACAACCTGCTTGTCTGCCACGTGACRGATTTCTACCCAGGCAGCATTCAAGTCCGATG
GTTCCTGAATGGACAGG;
(SEQ ID NO: 18)
AGGAAACAGCTGGGGTCGTGTCCACCAACCTGATCCGTAATGGAGACTGGACCTTCCAGATCCTGGTGATGCT
GGAAATGACCCCCCAGCA;
(SEQ ID NO: 19)
TCCCCATCCAGGACAGAGGCCCTCAACCACCACAACCTGCTGRTCTGCTCRGTGACAGATTTCTATCCAGSCCA
GATCAAAGTCCGGTGGTTTCGG;
(SEQ ID NO: 20)
AATGRYCAGGAGGAGACARCYGGCGTTGTGTCCACCCCCCTTATTAGGAAYGGTGACTGGACCTTCCAGATCCT
GGTGATGCTGGAAATGACTCCCCA;
(SEQ ID NO: 21)
GGGAAGACCACCTTTTCCGCAAGTTCCACTATCTCCCCTTCCTGCCCTCAACTGAGGACGTTTACGACTGCAGG
GTGGAGCACTGGGGCTTGG;
(SEQ ID NO: 22)
ATGAGCCTCTTCTCAAGCACTGGGAGTTTGATGCTCCAAGCCCTCTCCCAGAGACTACAGAGAAYGTGGTGTGT
GCCCTGGGCCTGACTGTGGG;
(SEQ ID NO: 23)
TKCAAAGACCCAGCCCCTGCAGCACCACAACCTCCTGGTCTGYTCTGTGARTGGTTTCTATCCAGGCAGCATTG
AAGTCAGGTGGTTCCKGAAY;
(SEQ ID NO: 24)
RGCCAGGAAGAGAAGRCTGGGGTGGTGTCCACRGGCCTGATCCASAATGGAGACTGGACCTTCCAGACCCTG
GTGATGCTGGAAACAGTTCCTCGG;
(SEQ ID NO: 25)
GCGGAGCTGGAGCGGAGAAGAGTCCAGGAATRCAAACAGCCAGAAGAACCTCTTGGGCTGCTTGCGGGGTCT
GTTGGACACCTACTGCAGACA;
(SEQ ID NO: 26)
GCCCTGACACACTGGATTGGAGAGCAGCAGAACCCAGGGCCTGGCCCACCAAGCTGGAGTGGGAAAGGCACA
AGATTCGGGCCAGGCAGAACAGG;
(SEQ ID NO: 27)
GCCTACCTGGAGAGGGACTGCCCTGCACAGCTGCAGCAGTTGCTGGAGCTGGGGAGAGGTGTTTTGGACCAA
CAAGTGCCTCCTTTGGTGAAGGTGA;
(SEQ ID NO: 28)
AGTCAGGGTTTCTYRCTGAGGTACATCTGGATGGTCAGCCCTTCCTGCGCTRTGACAGGCAGAAAYGCAGGGC;
(SEQ ID NO: 29)
AAAGCCCCAGGGACAGTGGGCAGAAGATGTCCTGGGAAATAAGACATGGGACAGAGAGACCAGRGACTTGACA
GGGA;
(SEQ ID NO: 30)
TGGGGATCAGTGTCCCTCACCATGGTCACCCTGRTCACCCTGCCTCTGCTTTTCCTTCTGCCCAAGAAGGTGGG
AAAATGGTACCAGTTGCTGG;
(SEQ ID NO: 31)
AAGTGCAGGTGCGGGAATCTCTGGCAAAGTCCAGCCAGGTGGCCATTGAGGYYCTGTCGGCCATGCCTACAGT
TCGAAGCTTTGCCAACGAGGAGG;
(SEQ ID NO: 32)
GCCGTTGGAGGGCTGCAGACCGTTCGCAGTTTTGGGGCCGAGGAGCATGAAGTCTGTCGCTATAAAGAGGCCC
TTGAACAATGTCGGCAGC;
or
(SEQ ID NO: 33)
TGTATTGGCGGAGAGACCTGGAACGCGCCTTGTACCTGCTCRTAAGGAGGGTGCTGCACTTGGGKGTGCAGAT
GCTGATGCTGAGCTGTGG

wherein R=A/G, Y=C/T, M=A/C, K=G/T, S=C/G, W=NT, B=C/G/T, D=AG/T, H=A/C/T, V=A/C/G, andN=NC/G/T
   or a sequence having at least 60%, 65%, 70%, 75%, 80%, 85%, 90%, 95%, 96%, 97%, 98%, or 99% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, or SEQ ID NO: 33.
63. The kit of any of embodiments 44-62, including a sequence having 95%, 96%, 97%, 98%, or 99% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, or SEQ ID NO: 33.
64. The kit of any of embodiments 44-63, including a sequence having the sequence of SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, or SEQ ID NO: 69 wherein R=A/G, Y=C/T, M=A/C, K=G/T, S=C/G, W=A/T, B=C/G/T, D=AG/T, H=A/C/T, V=A/C/G, and N=A/C/G/T
   or a sequence having at least 60%, 65%, 70%, 75%, 80%, 85%, 90%, 95%, 96%, 97%, 98%, or 99% sequence identity to SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, or SEQ ID NO: 69.
65. The kit of any of embodiments 44-64, having 95%, 96%, 97%, 98%, or 99% sequence identity to SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, or SEQ ID NO: 69.
66. The kit of any of embodiments 44-65, including blocking oligos including a sequence: CTACACGACGCTCTTCCGATCTIIIIIIIIIIIIIIIIIIIIIIIIIITTTCTTATATGGG (SEQ ID NO: 34); AAAAAAAAAAAAAAAAAAAAGTACTCTGCGTTGATACCACTGCTT (SEQ ID NO: 35); AAGCAGTGGTATCAACGCAGAGTACATGGG/3SpC3/(SEQ ID NO: 70); or AAGCAGTGGTATCAACGCAGAGTACATGGGAAAAAAAAAIAAAAAAAAAAIIIIIIIIIIIIIIIIIIIIIIIIAGATCGGAAGAGC GTCGTGTAG (SEQ ID NO: 36) or a sequence having at least 60%, 65%, 70%, 75%, 80%, 85%, 90%, 95%, 96%, 97%, 98%, or 99% sequence identity to SEQ ID NO: 34, SEQ ID NO: 35, or SEQ ID NO: 36.
67. The kit of any of embodiments 44-66, including blocking oligos including a sequence, wherein the blocking oligos include a sequence having 95%, 96%, 97%, 98%, or 99% sequence identity to SEQ ID NO: 34, SEQ ID NO: 35, or SEQ ID NO: 70, SEQ ID NO: 36.
68. The kit of any of embodiments 44-67, wherein the probes are linked to a detectable label.
69. The kit of any of embodiments 44-68, wherein the probes are biotinylated.
70. The kit of any of embodiments 44-69, wherein the probes are linked to a detectable label at the 5′ end of each probe,
71. The kit of any of embodiments 44-70, further including an RNA polymerase.
72. The kit of any of embodiments 44-71, further including a DNA polymerase.
73. A hybridization probe having the sequence:

(SEQ ID NO: 1)
ACCAGGCCTGCAGGGGATGGAACCTTCCAGAAGTGGGCGKCTGTGGTGGTRCCTTCTGGASAGGAGCAGAGAT
ACACCTGCCATGTGCAGCATGAGG;
(SEQ ID NO: 2)
GTYTSCCCAAGCCCCTCACCCTGAGATGGGAGCYGTCTTCCCAGCCCACCATCCCCATCGTGGGCATCATTGCT
GGCCTGGTTCTCYTTGGAGCTGTGW;
(SEQ ID NO: 3)
GGCAGCTGTGGTGGTGCCTTCTGGAGAAGAGCAGAGATACACATGCCATGTACAGCATGAGGGGCTGCCRAAG
CCCCTCACCCTGAGATGGG;
(SEQ ID NO: 4)
ACYTGGGGTCCGACGGWCGCCTCMTCCGCGGGTATGAACAGTATGCCTACGATGGCAAGGATTACCTCGCCC
TGAACGAGGACCTGCGCTCCT;
(SEQ ID NO: 5)
GCGAGTGGATGATCCAGCAAATAGGGCCAAAACTTGATGGGAAAATCCCGGTGTCCAGAGGGTTTCCTATCGCT
GAAGTGTTCACGCTGAAGCCCCTG;
(SEQ ID NO: 6)
GAGTTTGGCAAGCCCAACACTTTGGTCTGTTTTGTCAGTAATCTCTTCCCACCCATGCTGACAGTGAACTGGCAG
CATCATTCCRTCCCTGTGGAAGG;
(SEQ ID NO: 7)
CCCCTCTTACGGGGACACTTACACCTGTGTGGTAGAGCACAYTGGGGCTCCTGAGCCCATCCTTCGGGACTGG
ACACCTGGGCTGTCCC;
(SEQ ID NO: 8)
CCATGCAGACCCTGAAGGTTTCTGTGTCTGCAGTGACTCTGGGCCTGGGCCTCATCATCTTCTCTCTTGGTGTG
ATCAGCTGGCGGAGAGCTGGCC;
(SEQ ID NO: 9)
GGGCCAGCCCAACATCCTCATCTGCATCGTGGACAACATCTTCCCCCCTGTGATCAATATCACCTGGCTRCGCA
ACGGCCAAACTGTCACTGAGGGAG;
(SEQ ID NO: 10)
TGGCCCAGACCAGCTTCTATTCCCAGCCTGACCATTTGTTCCGCAAGTTCCACTACCTGCCCTTCGTGCCCTCA
GCCGAGGACGTCTATGACTGCCA;
(SEQ ID NO: 11)
GGGATGTTTGTGGCATTGACCAAGCTGGGGCAGCCAGATGCTGAGCAGTGGAACAGCCGGCTGGATCTCTTGG
ARAGGAGCAGACAGGCCGTGG;
(SEQ ID NO: 12)
ATGGGGTCTGTAGACACAACTACAGGCTGGGCGCACCCTTCACTGTGGGGAGAAAAGTGCAACCAGAGGTGAC
AGTGTACCCAGAGAGGACCCC;
(SEQ ID NO: 13)
CCAGTGCTCAACGTCACRTGGCTGTGCAAYGGGGAGCYGGTCACTGAGGGTGTCGCTGAGAGCCTCTTCCTGC
CCAGAACAGATTACAGCT;
(SEQ ID NO: 14)
TCCACAAGTTCCATTACCTGACCTTTGTGCCCTCAGCAGAGGACKTCTATGACTGCAGGGTGGAGCACTGGGGC
TTGGACCAGCCGCTCCT;
(SEQ ID NO: 15)
AGGTGGAGCACTGGGGCCTGGAYSAGCCTCTTCTGAAACACTGGGAGCCTGAGATTCCAGCMCCTATGTCAGA
GCTCACAGAGACTKTGGTCTGY;
(SEQ ID NO: 16)
RCCCTGGGRTTGTCTGTGGGCCTCRTGGGCATTGTGGTGGGCACTGTCTTCATCATCCRAGGCCTGCGTTCAG
TTGGTGCTTCCAGACACCARGGG;
(SEQ ID NO: 17)
GGGCCCYTGCAGCACCACAACCTGCTTGTCTGCCACGTGACRGATTTCTACCCAGGCAGCATTCAAGTCCGATG
GTTCCTGAATGGACAGG;
(SEQ ID NO: 18)
AGGAAACAGCTGGGGTCGTGTCCACCAACCTGATCCGTAATGGAGACTGGACCTTCCAGATCCTGGTGATGCT
GGAAATGACCCCCCAGCA;
(SEQ ID NO: 19)
TCCCCATCCAGGACAGAGGCCCTCAACCACCACAACCTGCTGRTCTGCTCRGTGACAGATTTCTATCCAGSCCA
GATCAAAGTCCGGTGGTTTCGG;
(SEQ ID NO: 20)
AATGRYCAGGAGGAGACARCYGGCGTTGTGTCCACCCCCCTTATTAGGAAYGGTGACTGGACCTTCCAGATCCT
GGTGATGCTGGAAATGACTCCCCA;
(SEQ ID NO: 21)
GGGAAGACCACCTTTTCCGCAAGTTCCACTATCTCCCCTTCCTGCCCTCAACTGAGGACGTTTACGACTGCAGG
GTGGAGCACTGGGGCTTGG;
(SEQ ID NO: 22)
ATGAGCCTCTTCTCAAGCACTGGGAGTTTGATGCTCCAAGCCCTCTCCCAGAGACTACAGAGAAYGTGGTGTGT
GCCCTGGGCCTGACTGTGGG;
(SEQ ID NO: 23)
TKCAAAGACCCAGCCCCTGCAGCACCACAACCTCCTGGTCTGYTCTGTGARTGGTTTCTATCCAGGCAGCATTG
AAGTCAGGTGGTTCCKGAAY;
(SEQ ID NO: 24)
RGCCAGGAAGAGAAGRCTGGGGTGGTGTCCACRGGCCTGATCCASAATGGAGACTGGACCTTCCAGACCCTG
GTGATGCTGGAAACAGTTCCTCGG;
(SEQ ID NO: 25)
GCGGAGCTGGAGCGGAGAAGAGTCCAGGAATRCAAACAGCCAGAAGAACCTCTTGGGCTGCTTGCGGGGTCT
GTTGGACACCTACTGCAGACA;
(SEQ ID NO: 26)
GCCCTGACACACTGGATTGGAGAGCAGCAGAACCCAGGGCCTGGCCCACCAAGCTGGAGTGGGAAAGGCACA
AGATTCGGGCCAGGCAGAACAGG;
(SEQ ID NO: 27)
GCCTACCTGGAGAGGGACTGCCCTGCACAGCTGCAGCAGTTGCTGGAGCTGGGGAGAGGTGTTTTGGACCAA
CAAGTGCCTCCTTTGGTGAAGGTGA;
(SEQ ID NO: 28)
AGTCAGGGTTTCTYRCTGAGGTACATCTGGATGGTCAGCCCTTCCTGCGCTRTGACAGGCAGAAAYGCAGGGC;
(SEQ ID NO: 29)
AAAGCCCCAGGGACAGTGGGCAGAAGATGTCCTGGGAAATAAGACATGGGACAGAGAGACCAGRGACTTGACA
GGGA;
(SEQ ID NO: 30)
TGGGGATCAGTGTCCCTCACCATGGTCACCCTGRTCACCCTGCCTCTGCTTTTCCTTCTGCCCAAGAAGGTGGG
AAAATGGTACCAGTTGCTGG;
(SEQ ID NO: 31)
AAGTGCAGGTGCGGGAATCTCTGGCAAAGTCCAGCCAGGTGGCCATTGAGGYYCTGTCGGCCATGCCTACAGT
TCGAAGCTTTGCCAACGAGGAGG;
(SEQ ID NO: 32)
GCCGTTGGAGGGCTGCAGACCGTTCGCAGTTTTGGGGCCGAGGAGCATGAAGTCTGTCGCTATAAAGAGGCCC
TTGAACAATGTCGGCAGC;
or
(SEQ ID NO: 33)
TGTATTGGCGGAGAGACCTGGAACGCGCCTTGTACCTGCTCRTAAGGAGGGTGCTGCACTTGGGKGTGCAGAT
GCTGATGCTGAGCTGTGG

wherein R=A/G, Y=C/T, M=A/C, K=G/T, S=C/G, W=A/T, B=C/G/T, D=A/G/T, H=A/C/T, V=A/C/G, andN=A/C/G/T
   or a sequence having at least 60%, 65%, 70%, 75%, 80%, 85%, 90%, 95%, 96%, 97%, 98%, or 99% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, or SEQ ID NO: 33.
74. A hybridization probe having 95%, 96%, 97%, 98%, or 99% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, or SEQ ID NO: 33.
75. A hybridization probe having the sequence of SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, or SEQ ID NO: 69 wherein R=A/G, Y=C/T, M=A/C, K=G/T, S=C/G, W=A/T, B=C/G/T, D=A/G/T, H=A/C/T, V=A/C/G, and N=A/C/G/T
   or a sequence having at least 60%, 65%, 70%, 75%, 80%, 85%, 90%, 95%, 96%, 97%, 98%, or 99% sequence identity to SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, or SEQ ID NO: 69.
76. A hybridization probe having 95%, 96%, 97%, 98%, or 99% sequence identity to SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, or SEQ ID NO: 69.
77. A blocking oligo having the sequence: CTACACGACGCTCTTCCGATCTIIIIIIIIIIIIIIIIIIIIIIIIIITTTCTTATATGGG (SEQ ID NO: 34); AAAAAAAAAAAAAAAAAAAAGTACTCTGCGTTGATACCACTGCTT (SEQ ID NO: 35); AAGCAGTGGTATCAACGCAGAGTACATGGG/3SpC3/(SEQ ID NO: 70); or AAGCAGTGGTATCAACGCAGAGTACATGGGAAAAAAAAAIAAAAAAAAAAIIIIIIIIIIIIIIIIIIIIIIIIIIIIAGATCGGAAGAGC GTCGTGTAG (SEQ ID NO: 36) or a sequence having at least 60%, 65%, 70%, 75%, 80%, 85%, 90%, 95%, 96%, 97%, 98%, or 99% sequence identity to SEQ ID NO: 34, SEQ ID NO: 35, or SEQ ID NO: 36.
78. A blocking oligo having 95%, 96%, 97%, 98%, or 99% sequence identity to SEQ ID NO: 34, SEQ ID NO: 35, or SEQ ID NO: 70, SEQ ID NO: 36.

### (ix) Experimental Examples

Example 1. Quantifying HLA transcripts by genotype in chimeric mixtures at single-cell resolution. Abstract. Gene products from the highly variable major histocompatibility locus, including HLA, are essential for self-recognition and immune surveillance of malignancy. Following allogeneic hematopoietic cell transplantation (alloHCT), genetic and epigenetic alterations in HLA can drive disease recurrence, making precise HLA assessment critical for determining future therapy. However, current methods lack the sensitivity to quantify HLA transcripts at the single-cell level, limiting their clinical utility. The current experiment introduces scrHLA-typing, which is a novel technique that accurately identifies and quantifies HLA transcripts in single cells using long-read sequencing. When applied to samples from patents with post-transplant relapse, scrHLA-typing successfully detected HLA allele-specific expression, across a range of levels of donor-recipient chimerism, at clinically actionable levels. By characterizing allele expression in residual leukemia cells, the assay identified differences in expression patterns among patients. This capability highlights scrHLA-typing's potential to improve risk stratification and guide the selection of appropriate salvage therapies, enhancing personalized treatment strategies after relapse.

Main. The human leukocyte antigen (HLA) genes reside in the extended major histocompatibility complex (MHC) region on chromosome 6p21.3, one of the most gene-dense and polymorphic stretches of the human genome (Horton, et al., Nat. Rev. Genet., 5, 889-899 (2004)). By April 2025, 42,584 HLA alleles had been catalogued across 46 clinically relevant genes in this region (Robinson & Marsh, HLA, 103, e15549 (2024); Barker, et al., Nucleic Acids Res., 51, D1053-D1060 (2023)). Variants in these loci account for more heritable disease than all other loci combined (Trowsdale & Knight, Annu. Rev, Genomic Hum. Genet., 14, 301-323 (2013); Matzaraki, et al., Genome Biol. 18, 76 (2017)). By presenting peptide antigens to T cells, HLA molecules are central to recognizing self vs. non-self, and this to the adaptive immune response against pathogens, alloantigens, and neoantigens in cancer (Dendrou, et al., Nat. Rev. Immunol. 18, 325-339 (2018)).

In allogenic hematopoietic cell transplantation (alloHCT), the effects of the patient and donor HLA disparity on graft rejection, graft-vs.-host disease, and survival post-HCT are well established (7,8). Importantly, when used as consolidation therapy for acute leukemias, alloHCT confers a beneficial graft-vs.-leukemia effect, in which HLA molecules play a crucial role (9). Indeed, a frequent mechanism of immune escape and relapse post-alloHCT involves the loss of HLA surface expression on leukemic cells (Toffalori, et al., Nat. Med., 25, 603-611 (2019); Rovatti, et al., Front. Immunol., 11, 147 (2020)). This alteration can result from epigenetic silencing of HLA or downregulation of regulators such as the CIITA (Matthew, et al., N. Engl. J. Med., 379, 2330-2341 (2018)). Alternatively, a loss of HLA heterozygosity (LOH) has been well documented in mismatched alloHCT in the direction of HLA mismatch (Vago Luca, et al., N. Engl. J. Med., 361, 478-488 (2009); Pagliuca, et al., Nat. Commun., 14, 3153 (2023)), and even observed in matched alloHCT (Jan, et al., Blood Adv., 3, 2199-2204 (2019). Generally, LOH of HLA is a well-described phenomenon in cancer, including outside the alloHCT context (McGranahan, et al., Cell, 171, 1259-1271.e11 (2017), highlighting a broad link between allele-specific HLA loss and cancer progression.

Current genomic methods for HLA typing are predominantly performed on DNA obtained from bulk cell populations. While computational methods exist to infer HLA types and expression levels from bulk (Bauer, et al., Brief. Bioinform., 19, 179-187 (2018); Orenbuch, et al., Oxf. Engl., 36, 33-40 (2020)) and single-cell RNA sequencing data (Darby, et al., Bioinformatics, 36, 3905, 3906 (2020); Solomon, et al., Front. Immunol., 14 (2023); Kang, et al., Nat. Genet., 55, 2255-2268, (2023)), no method currently enables the quantification of HLA molecules in complex mixtures. This limitation is particularly evident in settings such as post-alloHCT, where three or more alleles per gene can coexist at varying proportions—and often disproportionate levels. The high degree of homology among HLA genes and alleles (some differing by as little as a single nucleotide) introduces significant challenges, including variant call errors and alignment bias. These issues render traditional short-read sequencing pipelines inappropriate (Brandt, et al., G3 GenesGenomesGenetics, 5, 931-941 (2015) for use after transplantation. Consequently, most sequencing-based HLA genotyping approaches rely on statistical imputation of single nucleotide polymorphisms in linkage disequilibrium with known alleles (Sakaue, et al., Nat. Protoc., 18, 2625-2641 (2023), or by alignment to a reference of all known alleles, subsequently narrowing down to one (homozygous) or two (heterozygous) alleles per gene by statistical inference (Bai, et al., BMC Genomics, 15, 325 (2014); Shukla, et al., Nat. Biotechnol., 33, 1152-1158 (2015); Buchkovich, et al., Genome Med., 9, 86 (2017)) or iterative realignment to a personalized reference (Xie, et al., Proc. Natl. Acad. Sci., 114, 8059-8064 (2017); Aguiar, et al., PLOS Genet., 15, e1008091 (2019); Aguiar, et al., Methods Mol. Biol. Clifton NJ, 2120, 101-112 (2020), disregarding supernumerary alleles as biologically or methodologically implausible.

It is hypothesized that long-read sequencing of HLA transcripts in single cells could resolve the HLA landscape in individual cells, especially within complex mixtures such as those encountered after alloHCT. A sing-cell RNA-based HLA typing (scrHLA-typing) was developed: a novel method that combines molecular enrichment of single-cell barcoded HAL full transcripts with long-read sequencing to enhance allele typing accuracy. A dedicated computational pipeline performs genotype prediction, error-filtering, and single-cell quantification accommodating complex allelic multiplicity. Applying scrHLA-typing to bone marrow aspirates (BMAs) from patients with relapsed leukemia following alloHCT, this approach was demonstrated to robustly quantify differential HLA allele expression across diverse cell populations, providing clinically actional insights.

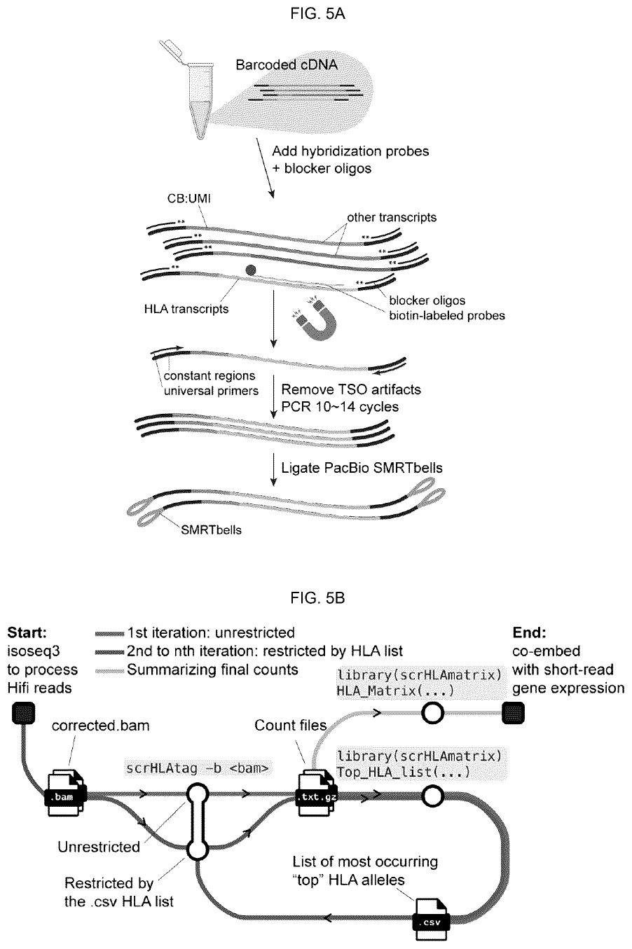

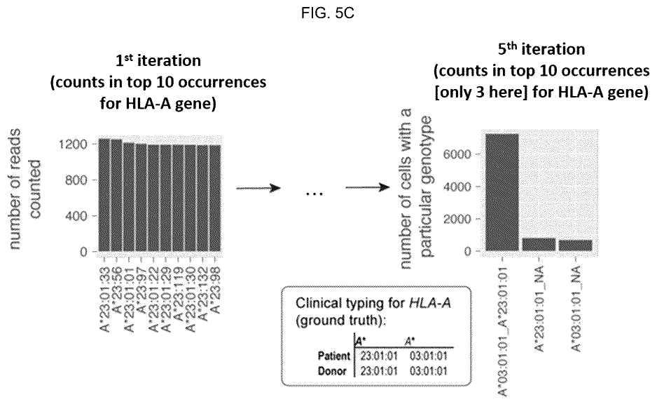

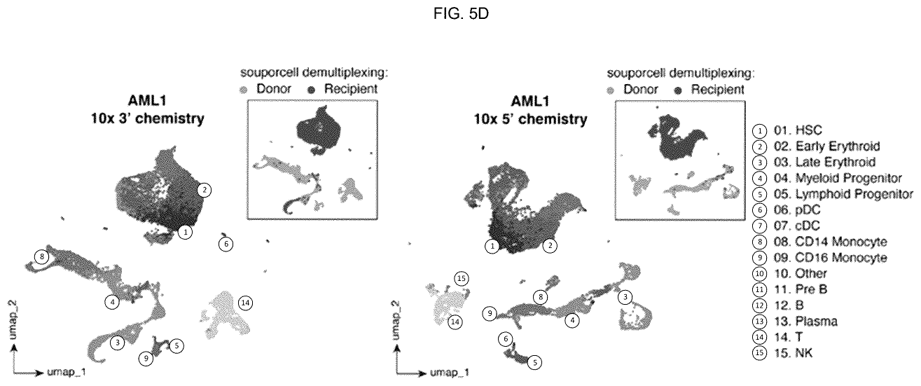

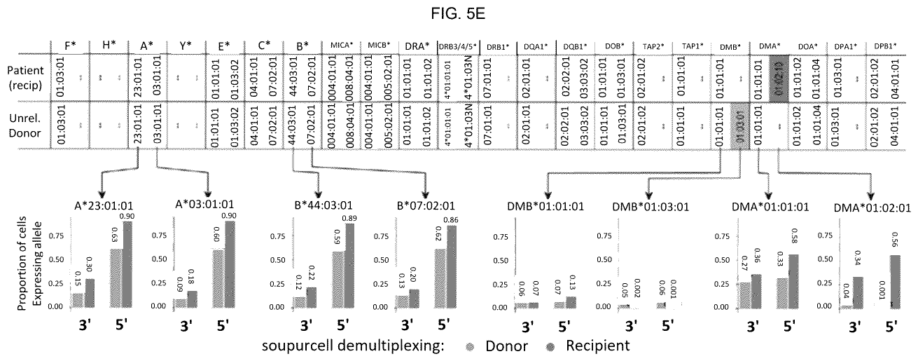

Results. Designing the scrHLA-typing workflow. The techniques used in this experiment rely on the generation of a single-cell barcoded whole transcriptome amplified pool (the cDNA pool) from a sample of cells, in which each cDNA molecule includes a cell-barcoded (CB) sequence (to mark single-cell provenance) and a unique molecular identified (UMI) sequence (to quantify and eliminate PCR duplication events). A panel of biotinylated DNA oligonucleotides (hybridization probes) were created that specifically recognize common regions of coding DNA sequence of classical and non-classical (including pseudogenes) HLA alleles in the IMGT/HLA database (Barker, et al., Nucleic Acids Res., 51, D1053-D1060 (2023); FIG. 5A). Although optimized for 10× genomics-derived cDNA, any single-cell barcoded whole transcriptome should be compatible. After hybridization capture, HLA fragments were then re-amplified and prepared specifically for PacBio long-read sequencing (FIG. 5A).

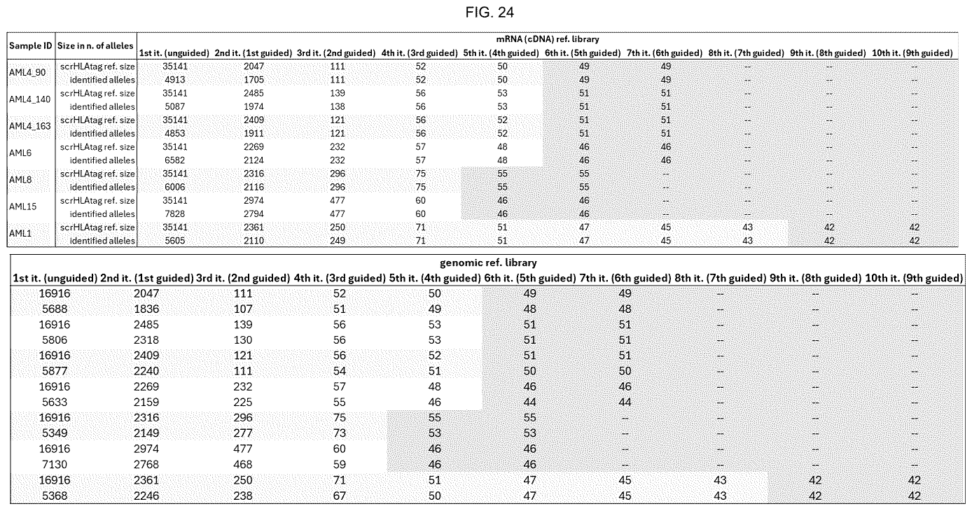

For data analysis, a pipeline with two components was developed. ‘scrHLAtag’, a command line tool, aligns reads to either the full or partial list of the IMGT/HLA reference (i.e., when HLA types are known). When HLA types are not known apriori, of “best-guess” list is iteratively generated combining scrHLAtag with ‘scrHLAmatrix’, an R package that detects HLA patterns, summarizes calls per cell, filters errors and produces data objects that integrate into current short-read analysis pipelines (FIG. 5B). In patient samples used throughout this study, scrHLAmatrix-to-scrHLAtag typically converged on the putative HLA genotypes after 5 to 9 iterations (FIG. 5C, FIG. 5E; FIG. 24, Table 1).

For the first experiment, a scrHLA-typing was performed on a gradient centrifuged BMA sample from an 11-year-old male patient with CD34+ acute myeloid leukemia (AML), collected 783 days after HLA-matched unrelated donor myeloablative alloHCT when low level relapse had been confirmed clinically using multiparameter flow cytometry. To boost numbers of leukemic blasts, a fraction of the sample was captured after performing CD34+ immunomagnetic enrichment. Single-cell RNA droplet capture of both unenriched and CD34+ cells was performed using both 10× Genomics 5′ and 3′ single-cell chemistries, followed by whole transcriptome short-read sequencing.

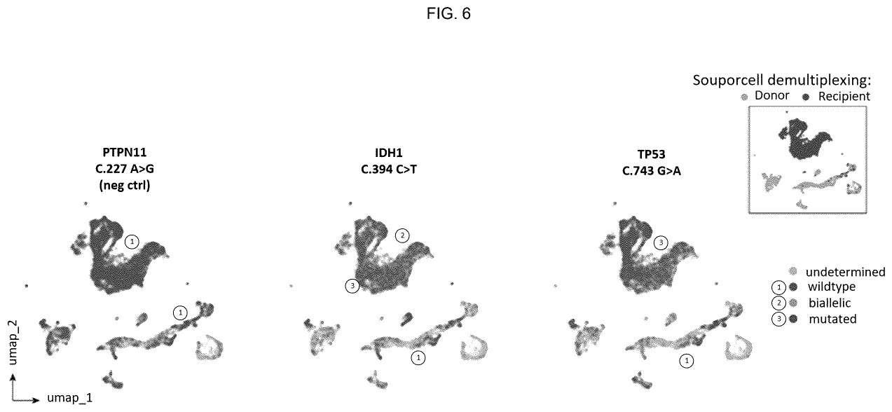

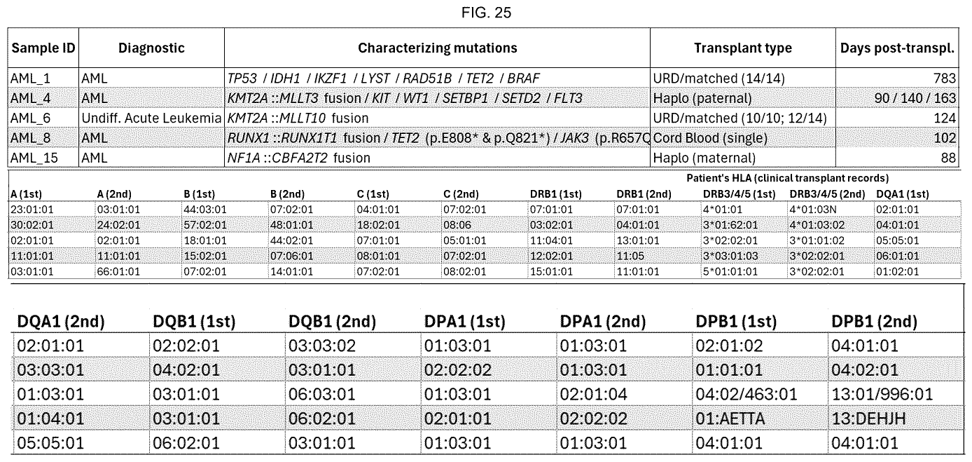

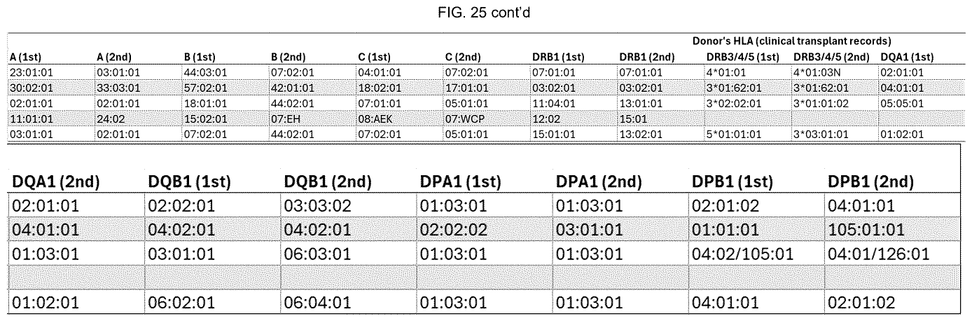

Souporcell, a genetic demultiplexing algorithm capable of distinguishing donor vs. recipient celsl using variants present in the aligned reads was used to establish “ground truth” (Heaton, et al., Nat. Methods, 17, 615-620 (2020)). ViewmastR (Krakow, et al., Blood, 114, 1069-1082 (2024)) was then used to annotate cell types according to a known reference, in this case, a well annotated atlas of health bone marrow mononuclear cells (Granja, et al., Nat. Biotechnol., 37, 1458-1465 (2019)). Putative leukemic “recipient” cells were annotated as hematopoietic stem cells, monocytes, and erythroid and myeloid progenitors in an overlapping continuum typical of dysregulated differentiation. In contrast, putative “donor” cells followed an orderly hierarchical pattern of moral hematopoietic differentiation (FIG. 5D). To unequivocally confirm the identity of these cells using an approach previously described (van Galen, et al., nat. Biotechnol., 37, 1458-1465 (2019)), the biotinylated hybridization probes specific to two of the genes know to be mutate in the leukemia of this patient were designed: TP53 and IDH1 (FIG. 25). As expected, mutated TP53 and IDH1 were present only in leukemia recipient cells (FIG. 6).

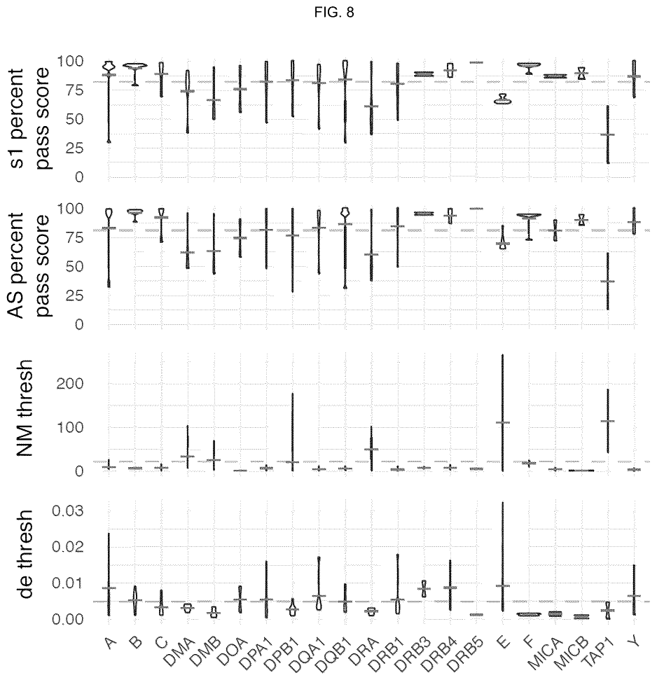

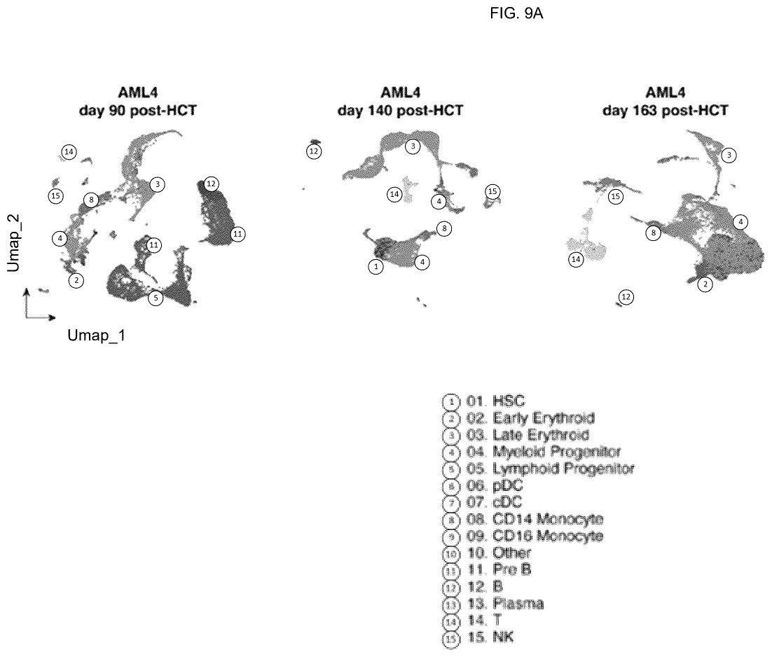

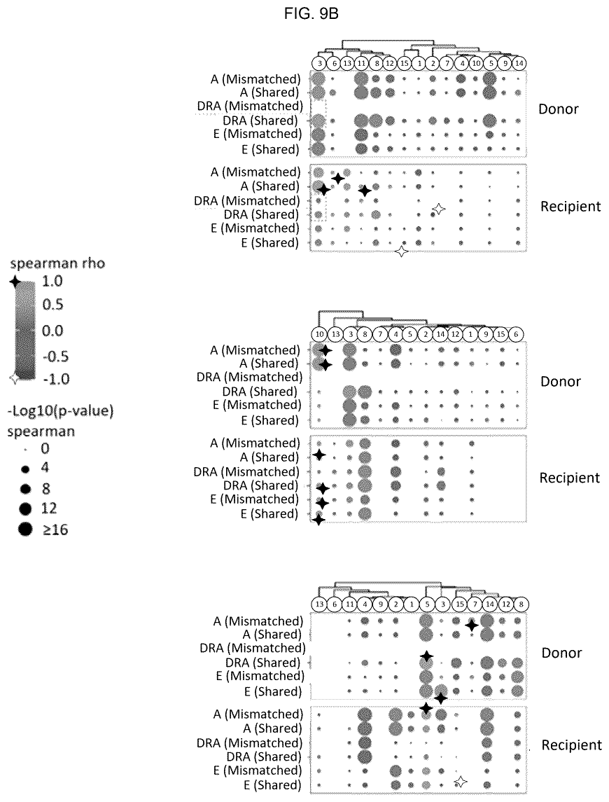

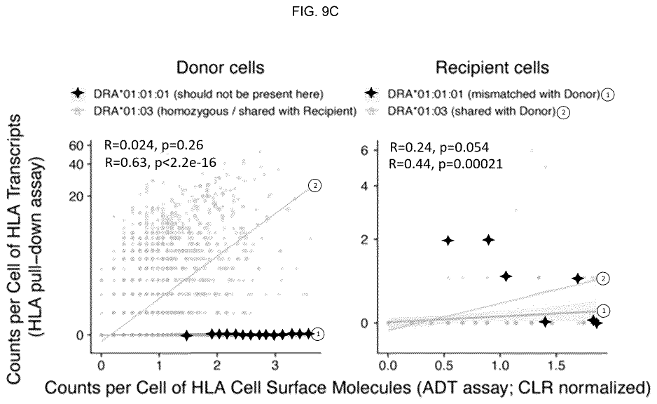

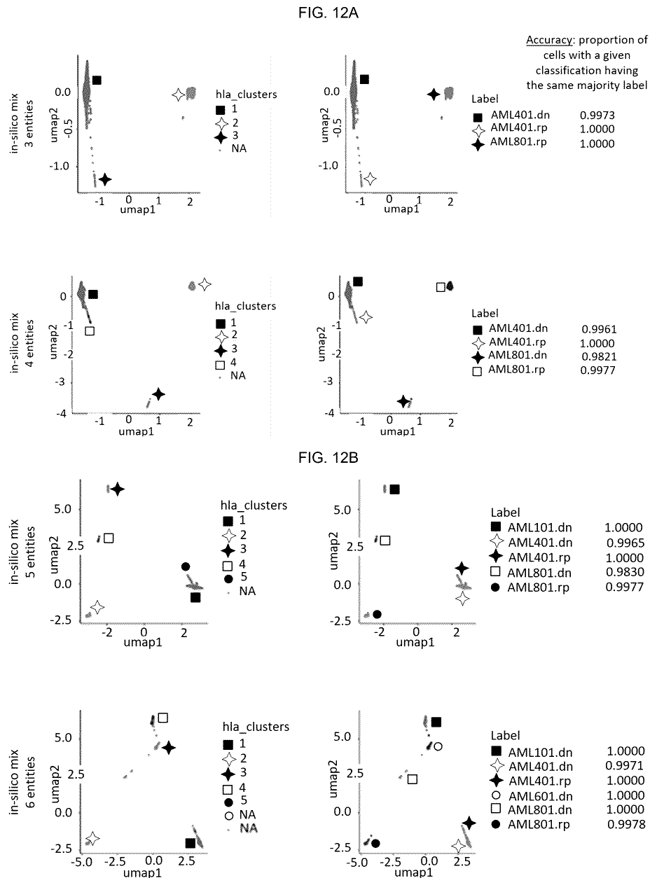

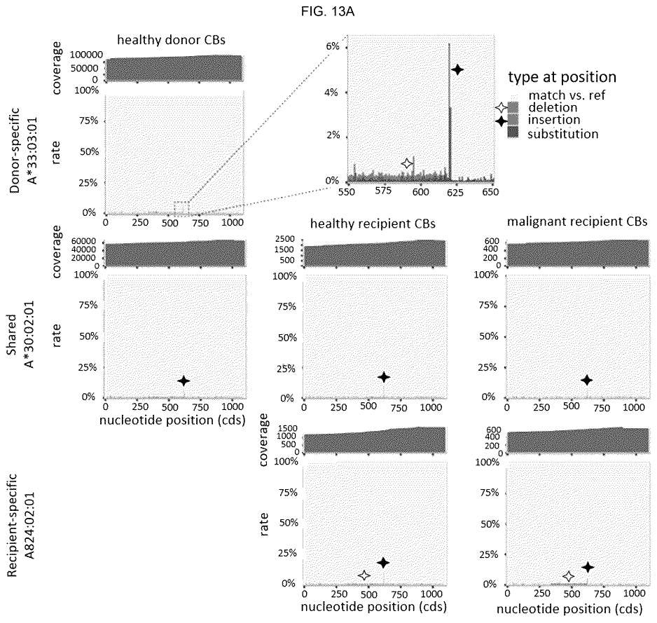

To generate HLA-to-CB count matrices that could be integrated with short-read profiles, the low-quality reads (based on aligner scores; FIG. 8) were filtered and then the PCR duplications (with additional post-deduplication corrections) were removed. Even after filtering for reads with low quality, the presence of reads (with identical CB and UMI) that contained amalgam sequences made up of 2 HLA alleles were noted. This phenomenon was attributed to a template-switching PCR artifact which has been described in sequencing applications (Kebschull & Zador, Nucleic Acids Res., 43, e143 (2015)), but is hypothesized to be more common when input libraries are less complex (FIG. 9A) such as when amplifying HLA sequences, where extensive polymorphism and inter-allele/inter-gene homology are defining features. As such, a deduplication strategy using “majority-wins” approach was adopted. To evaluate the robustness of the computation strategy for HLA-based cell classification, in silico mixing experiments were conducted using combinations of 3 to 6 post-alloHCT patient samples and achieved consistently high classification accuracy FIG. 12A; FIG. 12B).

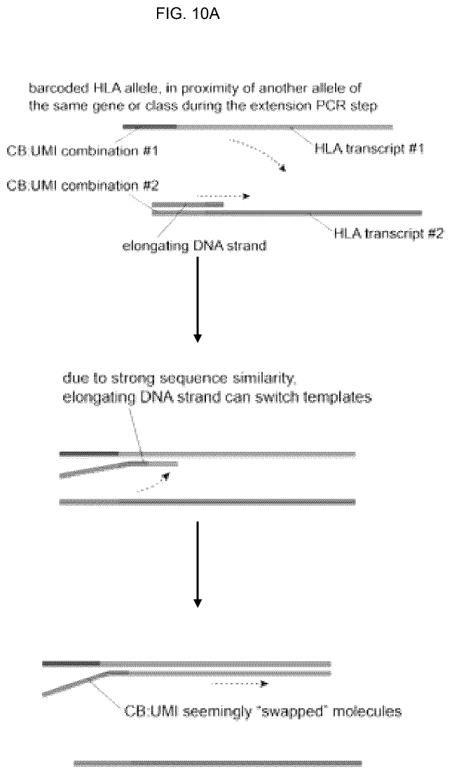

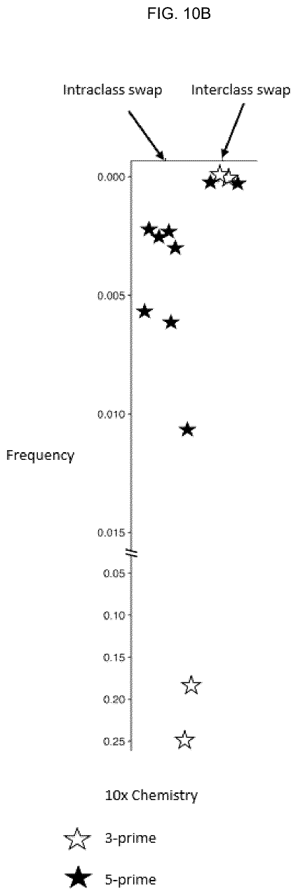

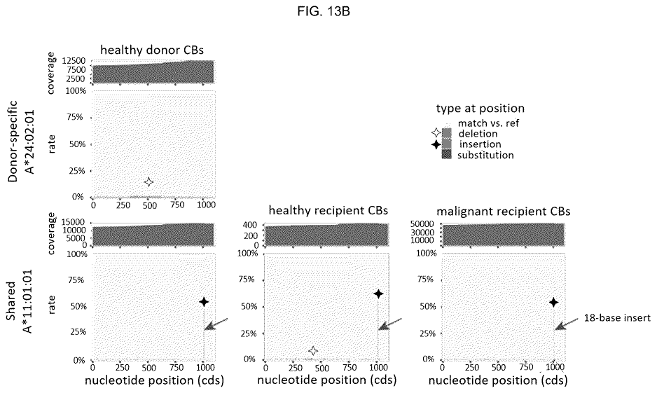

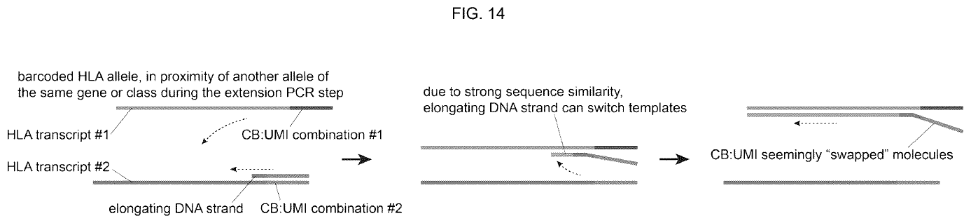

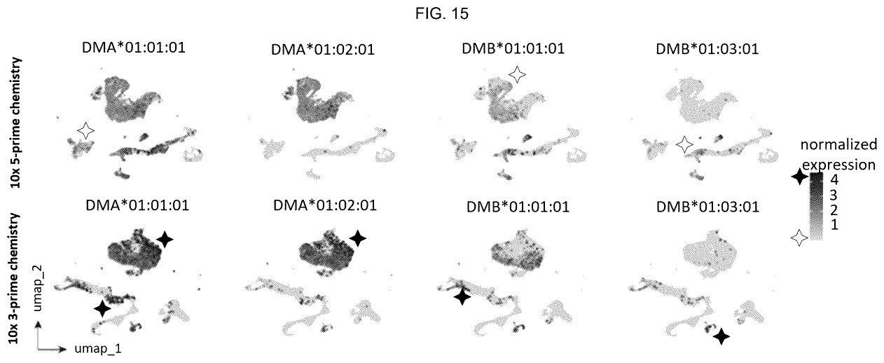

The next question was how per-cell read coverage and erroneous allele calls (i.e., donor-specific alleles in recipient cells and vice-versa, recipient-specific alleles in donor cells) compared between the 2 capture chemistries. It was found that the proportion of cells expressing any HLA allele was higher in the 5′ captures, with intra-class molecular swapping events 45 times smore common in 3′ captures (FIG. 10B) This may be explained by the position of hypervariable HLA regions on the 5′ side (FIG. 13A). Finally, among the only 2 non-shared alleles in an otherwise perfectly HLA-matched donor-to-recipient alloHCT (FIG. 5E; FIG. 15), the false positive rates (i.e., erroneous allele calls) were from 2 to 40-fold higher in 3′ vs. 5′ (FIG. 5E).

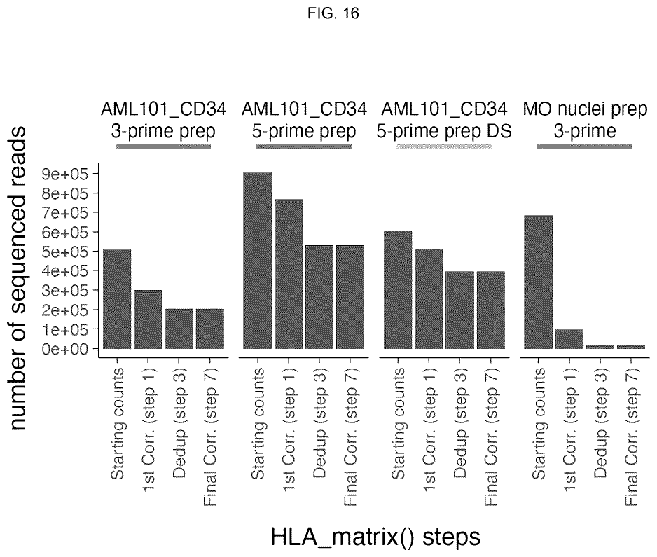

To broaden the potential usage of scrHLA-typing onto single nuclei preparations, experimentation was performed on cDNA from a sample of human T cells where others had captured in a co-assay of single-cell RNA and ATAC (Jaeger-Ruckstuhl, et al., Immunity, 57, 287-302.e12 (2024)), in which cells were lysed/permeabilized to allow nucleus tagmentation and barcoding while simultaneously barcoding RNA at the 3′ end. These nuclei captures had a dearth of HLA transcripts and very few reads passed quality control (FIG. 16). Given the superiority of pairing scrHLA-typing with 5′ RNA capture chemistry subsequent experiments using 5′ barcoded samples with intact cells was performed.

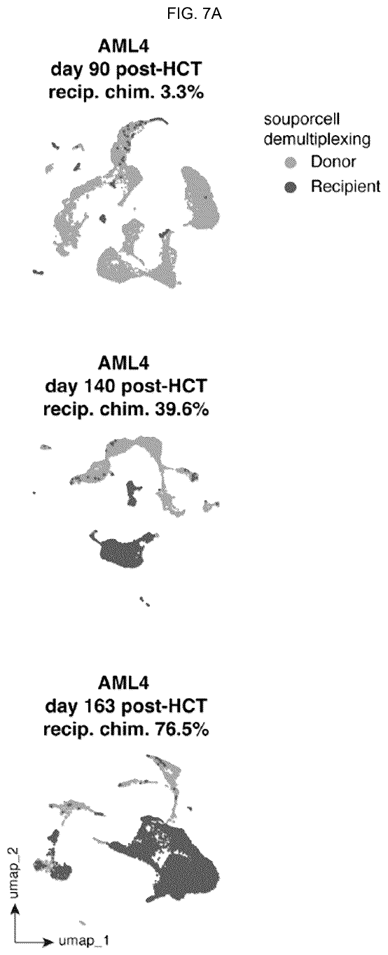

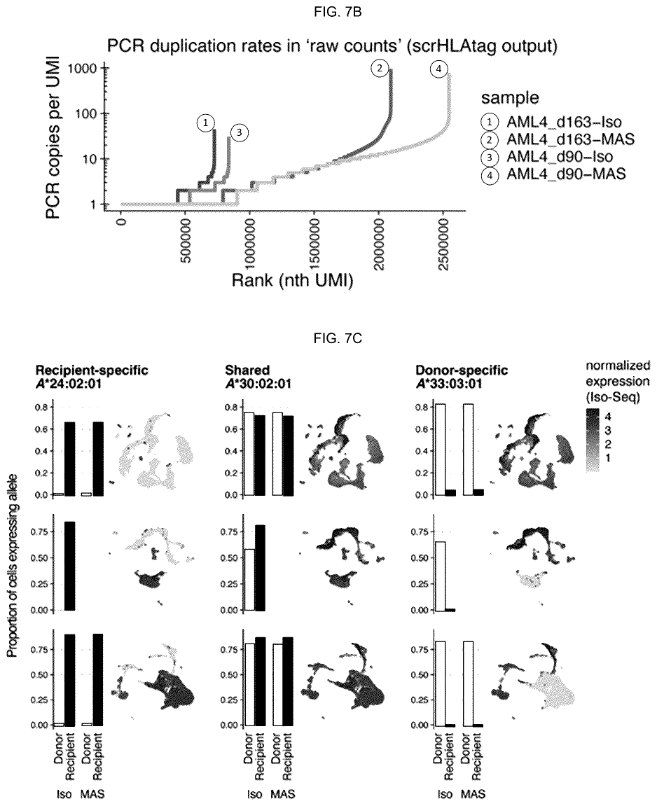

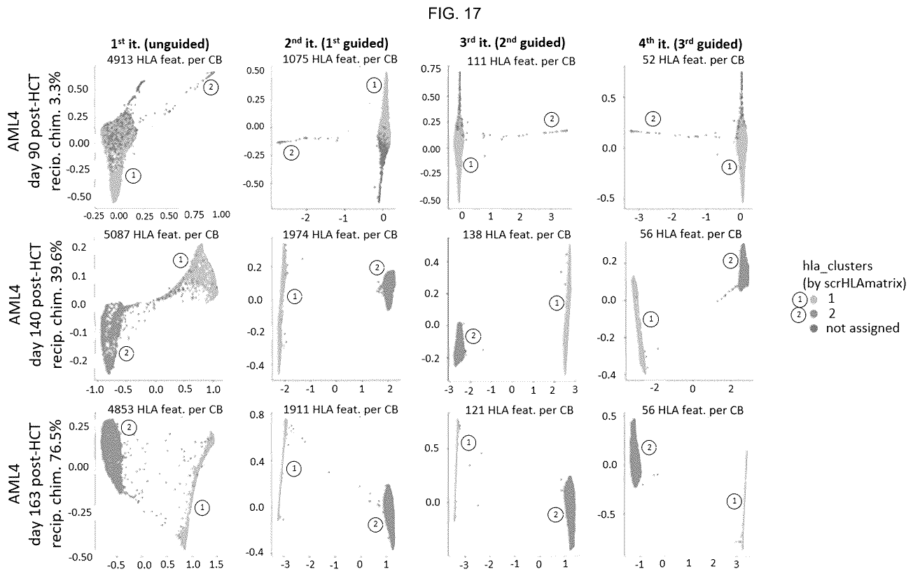

Quantifying HLA in samples with disproportionate or more balanced chimerism levels. To access scrHLA-typing across a range of disease burdens, samples were obtained from a 6-year-old patient with AML (AML4), collected post-haploidentical (donor source: father) myeloablative alloHCT at three timepoints. At day 90, when AML relapse was suspected clinically (0.04% burden by multiparameter flow cytometry), and at days 140 and 163, after over clinical relapse (11.5% and 43%, respectively). Genetic demultiplexing results reflected clinical burden of disease (FIG. 8). Remarkably, low level chimerism had no influence on the number of iterations needed for convergence (FIG. 17 and FIG. 24) Furthermore, scrHLA-typing results had high fidelity to genetic demultiplexing (FIG. 7B).

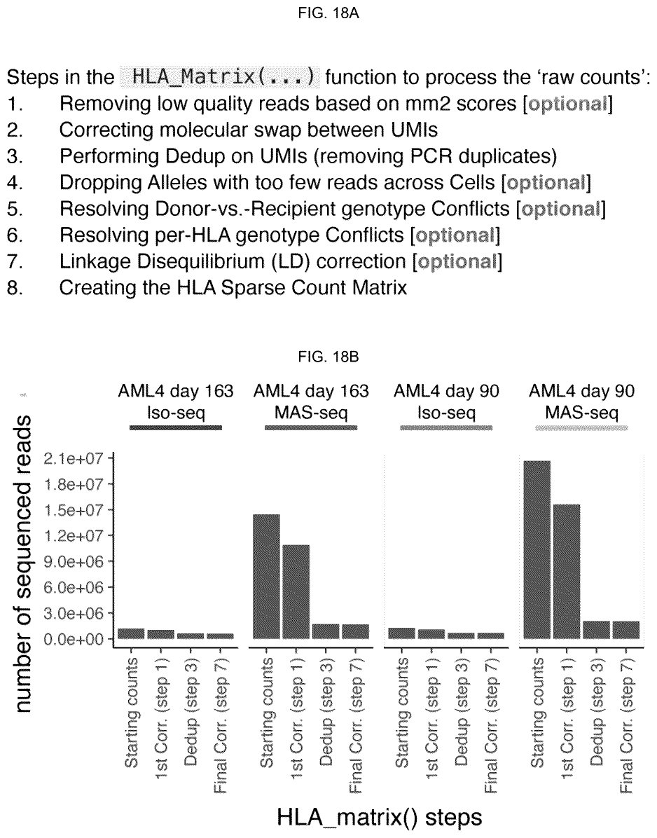

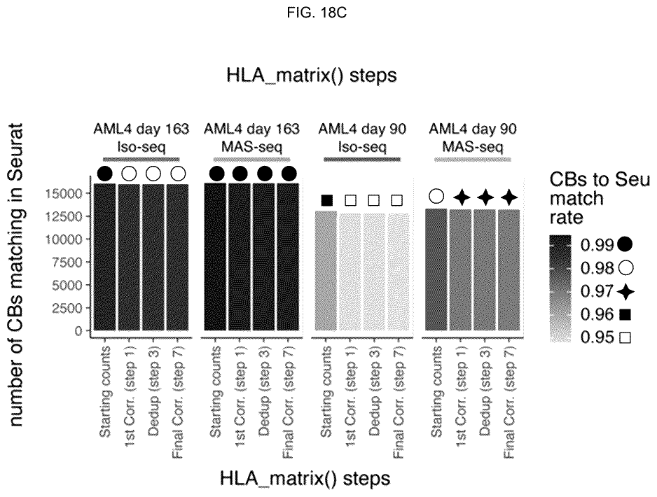

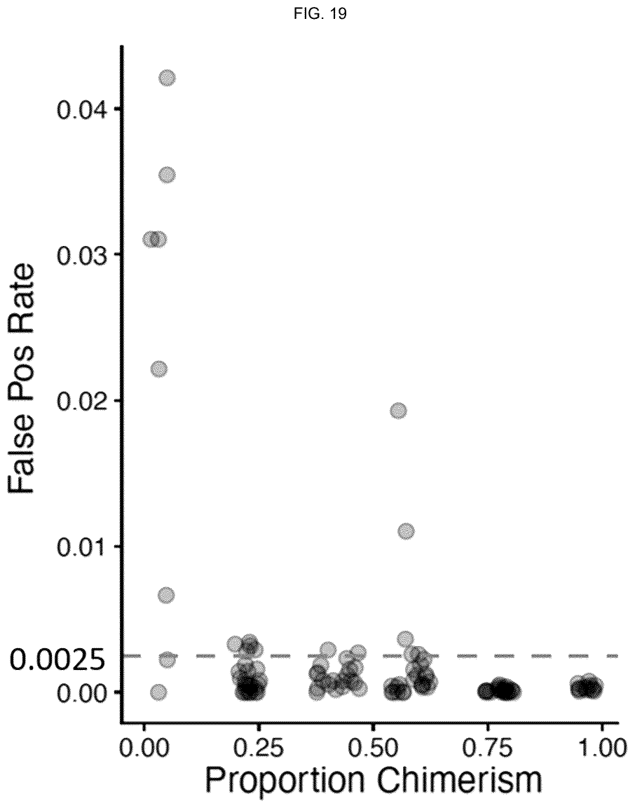

In order to establish whether increasing sequencing depth improved scrHLA-typing sensitivity, “multiplexed arrays isoform sequencing” (MAS-seq) (Liu, et al., Cell, 165, 535-550 (2016)) was employed, in parallel with the standard “Iso-Seq”, on the samples “AML4 day 80” and “AML4 day 163” (FIG. 18B). Although MAS-seq increased raw read output 12-16 fold vs. Iso-Seq, PCR-duplication rates were considerably higher (FIG. 7B). Cell barcode coverage remained comparable with Iso-Seq (FIG. 18C), normalized HLA expression patterns were similar, and the ability to resolve alleles for each allogeneic entity and the resulting erroneous allele calls (false positives) were almost indistinguishable from Iso-Seq (FIG. 7C), with a global average false positive rate at 0.25% for 5″ chemistry captures (FIG. 19). In all, results did not show significant benefit for using the higher throughput of MAS-seq across a range of donor chimerism levels, though it was still be considered for highly multiplexed experiments.

HLA transcript count correspondence with cell surface molecular expression. In an effort to assess whether quantification of HLA transcript correlated with protein level expression, pan HLA-A, -DRA, and -E on the cell surfaces using CITE-Seq (Stoeckius, et al., Nat. Methods, 14, 865-868 (2017)) for “AML4” samples was evaluated.

To account for the well-described effect of cell state (steady vs. in-transition, long-term commitment vs. short-term adaptation, differentiation status, etc.) on mRNA-to-protein correlation (Fan, et al., Cell Rep., 43, 113928 (2024); Abreu, et al., Mol. Biosyst., 5, 1512-1526 (2009)), cell types were considered as a proxy, and found heterogeneity in HLA allelic transcript-to-protein correspondence by cell type (FIG. 9A; FIG. 9B). In general, cell types clustered into 2 groups: exhibiting positive correction (example in FIG. 9C) or exhibiting no significant correlation and sometimes negative correlation suggesting transcript-to-protein “signal delay” or prolonged “persistence” of stable protein without its encoding transcript (FIG. 9B). Notably, in some cell types, for example the recipient-specific late erythroid progenitors for the 3 timepoints (example in FIG. 9C) and the recipient specific CD14+ monocyte-like cells for “AML4 day 90”, transcripts of one allele (e.g., the “shared” allele) had slightly stronger correlation with surface molecule expression vs. the second allele, suggesting a stronger contribution of the former rather than the latter to protein formation. This analysis reveals that the correlation between HLA allelic transcript counts, and corresponding cell surface protein quantities is cell type-dependent, and occasionally allele-dependent. This underscores the complexity of mRNA-to-protein stoichiometry, influenced by cell state and lineage.

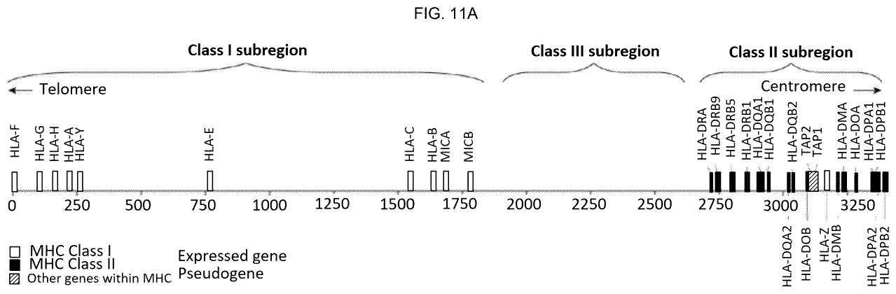

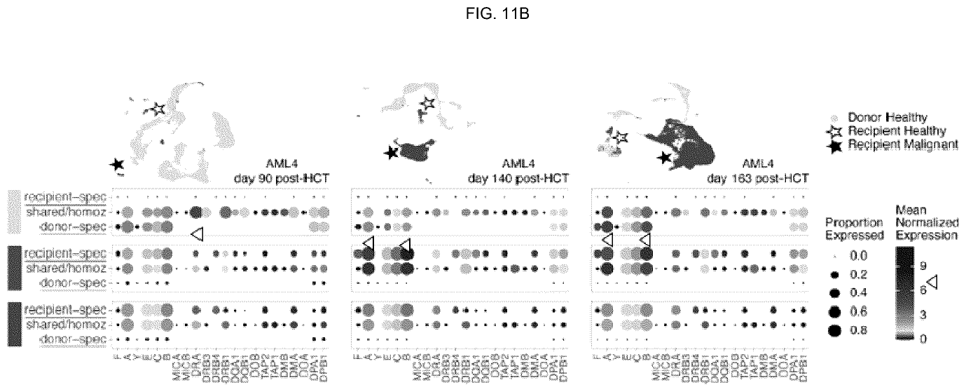

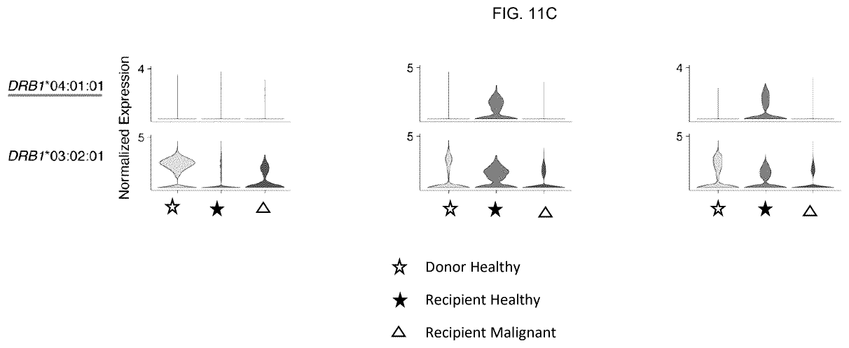

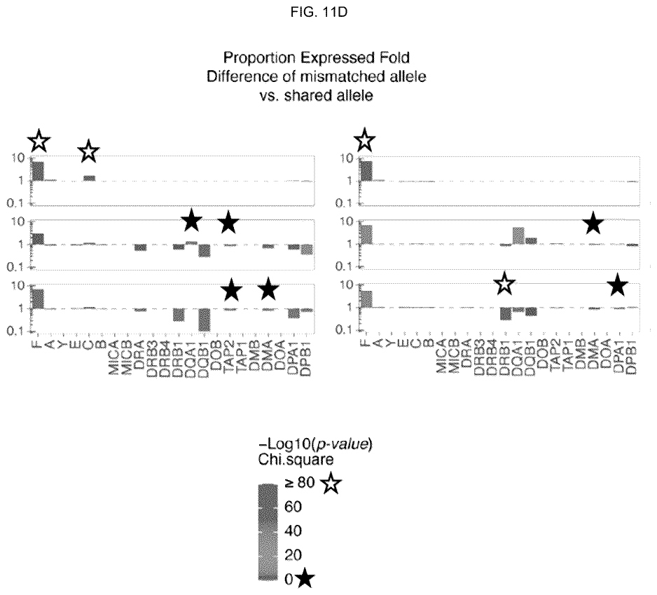

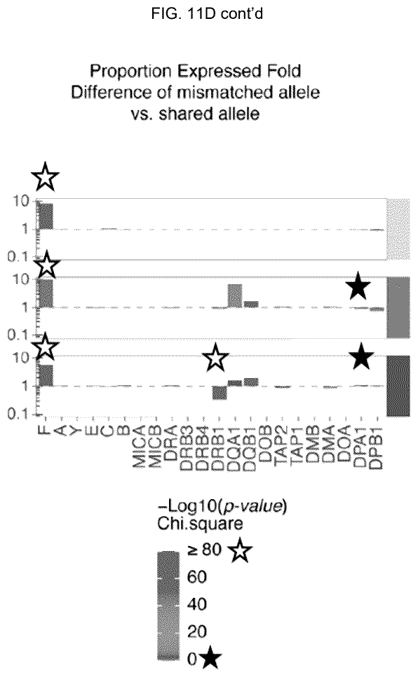

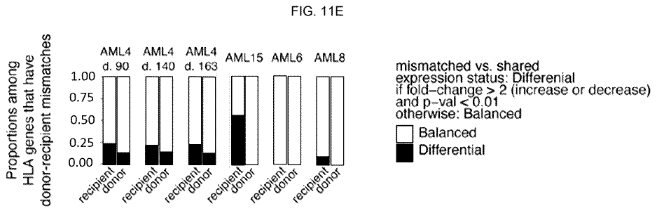

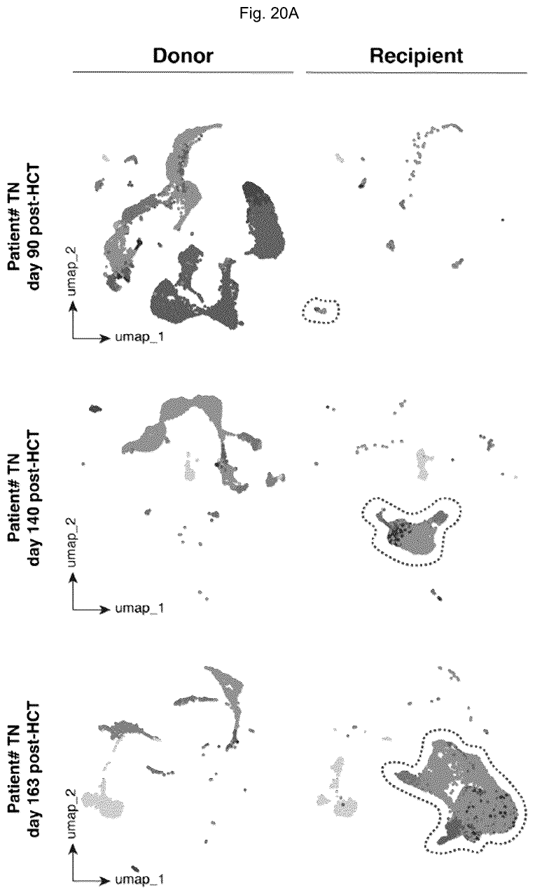

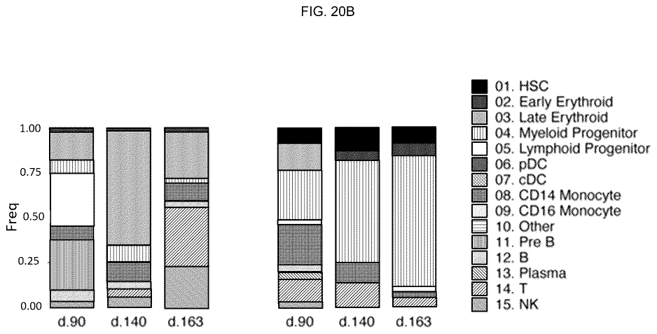

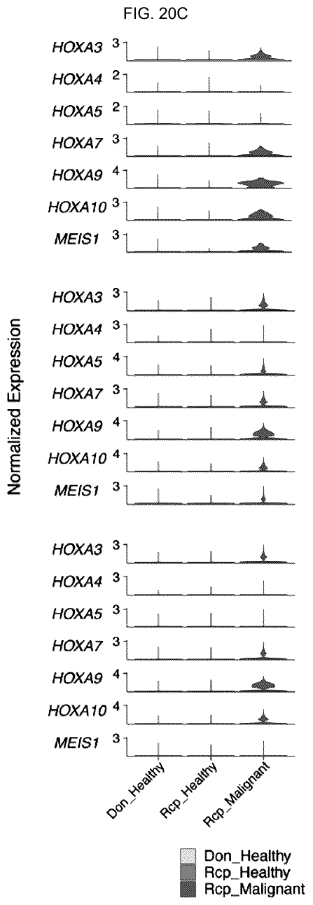

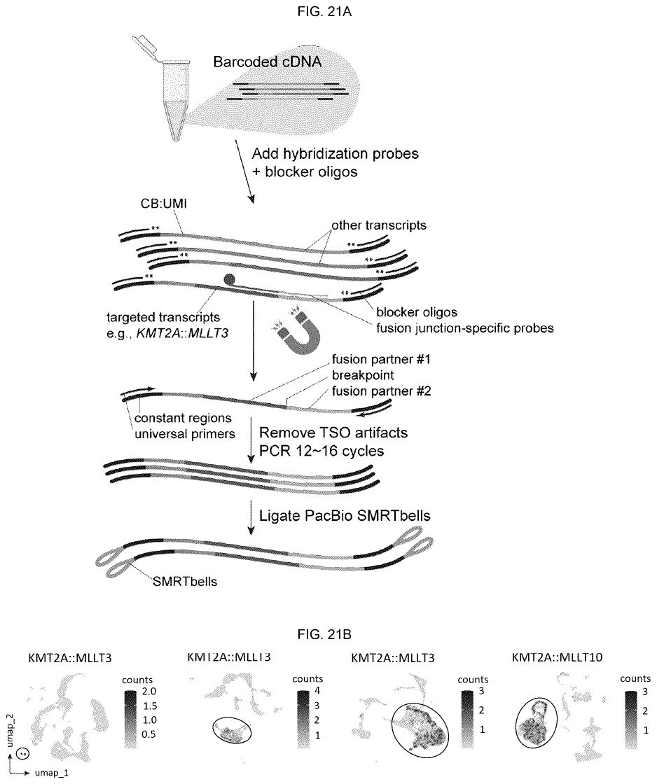

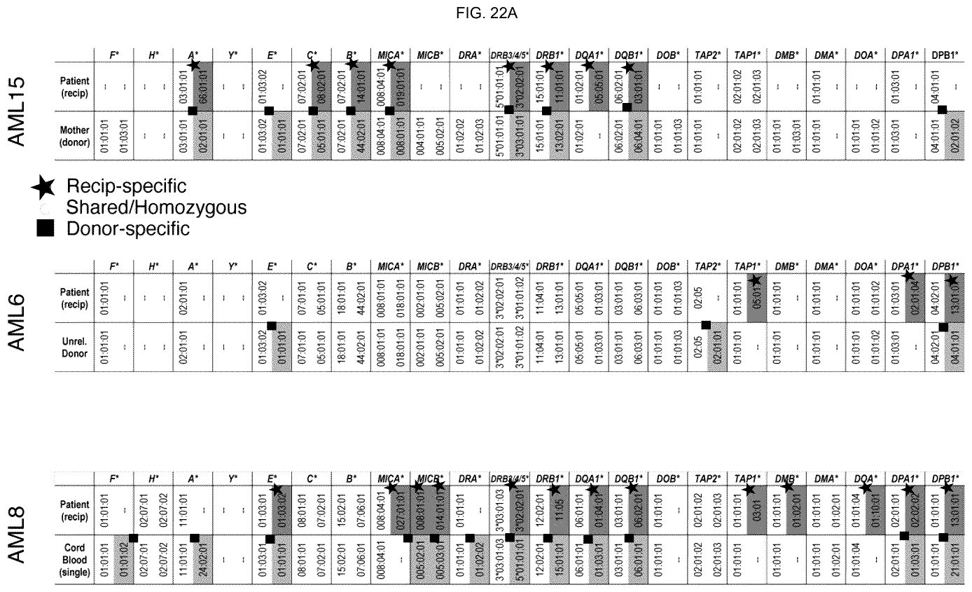

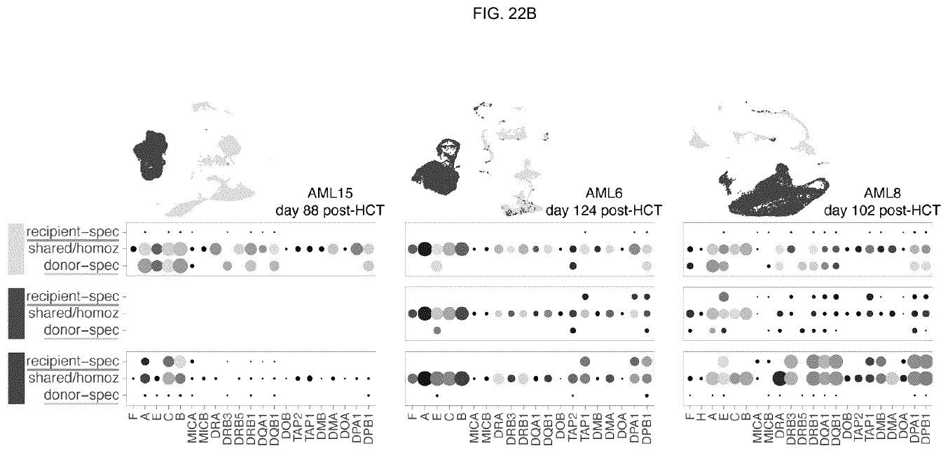

Differential HLA allele expression is present in leukemia cells of some but not all patients post-alloHCT. It was next asked whether mismatched HLA alleles (vs. shared) were differentially expressed longitudinally in “AML4” (FIG. 11A). Given that persistent recipient T cell chimerism was present in “AML4”, the recipient cells were further subset into “malignant” vs. “healthy”. Like in patient “AML1”, putative leukemia cells 1) exhibited evidence of disorganized differentiation (FIG. 20A), 2) expanded in numbers over time (FIG. 20B), 3) expressed genes implicated in cellular immorality (Alharbi, et al., Leukemia, 27, 1000-1008 (2013); FIG. 20C), and 4) expressed the AML-defining KMT2A::MLLT3 fusion (FIG. 21B) clinically detected in this patient (cryptic fusion difficult to detect, negative by karyotype and fluorescence in situ hybridization cytogenetic methods, eventually characterized by the FusionPlex (Chen, et al., J. Mol. Diagn., 25, 932-944 (2023)) and UW-OncoPlex (Pritchard, et al., J. Mol. Diagn., 16, 56-67 (2014); Kuo, et al., Pract. Lab. Med., 19, e00153 (2020)) molecular assays).

In all 3 groups: donor healthy, recipient healthy (mostly lymphocytes), and recipient malignant, class II genes exhibited a greater degree of unbalanced allele-specific expression (ASE) than class I (FIG. 11B; FIG. 11D). Among leukemic cells, some recipient-specific class II alleles were downregulated vs. their shared counterpart. In particular, the recipient-specific DRB1 allele (DRB1*04:01:01) was significantly downregulated compared to the shared (DRB1*03:02:01) in recipient malignant cells but not in recipient healthy cells (more obvious in later timepoints), suggesting a cell type-dependent rather than an allele intrinsic-dependent mechanism (FIG. 11C). Highly statistically significant unbalanced ASE in DRB1 was evident early and remained consistent across the 3 timepoints post-alloHCT (FIG. 11D).

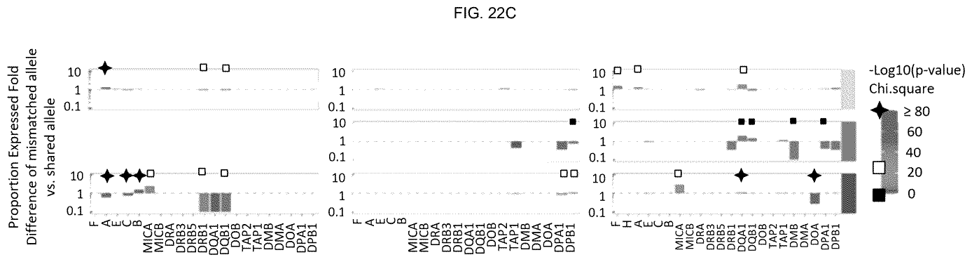

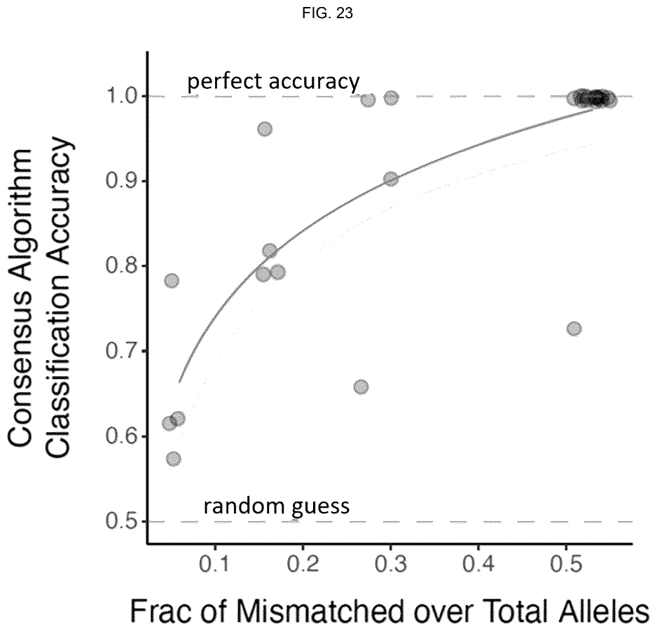

3 additionally post-alloHCT AML patients were tested for mismatched allelic downregulation within leukemic cells (FIG. 22A). Taking a similar approach to determine malignant cells as described above (using fusions if available), one patient, “AML15”, with recurrent disease post-haploidentical transplant (source: mother) had evidence of an erythroblastic leukemia (FIG. 22A) that also exhibited unbalanced ASE (FIG. 22B), strongest among class II alleles (FIG. 22C). In other patients, unbalanced ASE was not appreciable in leukemic cells; patients “AML6” and “AML8” with relapse post-alloHCT (source: matched unrelated and single cord blood, respectively), had little differential allele expression per HLA gene across the haplotypes. For “AML8”, only the recipient-specific intracellular-expressed HLA-DOA was significantly downregulated vs. shared (FIG. 22C).

Moreover, unlike recipient cells, the donor cells of all 4 patients generally maintained “balanced” HLA expression across the haplotypes, in line with the understanding that cells from the donor maintain alloimmunologic pressure after alloHCT (Koster, et al., Transplant. Cell. Ther., 29, 268.e1-268.e10 (2023)). Furthermore, ASE of HLA in setting of donor-recipient mismatch was always more prevalent in recipient cells compared with donor cells (FIG. 11E) using thresholds of an absolute fold-change of 2, and p-value<0.01. Altogether, the assay robustly detected ASE present in HLA genes. Some patients exhibited such differential expression in their leukemia cells, but others did not.

Assessing mismatch patterns in HLA sequencing reads across single cells. The next question was whether scrHLA-typing could identify sequence variation in the coding region of HLA. Reads that mapped correctly to their corresponding IMGT/HLA reference showed a high-fidelity rate, averaging a 99.7% match across all nucleotide positions, loci, and patient samples. Minor mismatches observed were evenly distributed across both loci and patient samples, likely attributable to technical artifacts from reverse transcription, PCR, and sequencing. An illustrative example of this low-level variation at the HLA-A locus in the ‘AML4 day 90’ sample is shown in FIG. 13A.

In contrast, a variant was identified in patient “AML8”, who was HLA-A*11:01:01 homozygous and had received a transplant from an A*11:01:01/A*24:02:01 donor. Specifically, an 18-base insertion at the 3′ end of the A*11:01:01 reads was observed, with a variant allele frequency of 47.4%. The single-cell approach uniquely enabled an evaluation of this insertion across different cell populations; looking across healthy donor, healthy recipient, and malignant recipient cells, the insertion remained consistent at approximately that same frequency, unlikely to be disease-specific (FIG. 13B). The insertion was further identified as a known alternative splice of HLA-A (FIG. 13C), resulting in an in-frame addition of 6 residues (GGEGVK) at position 314 at the cytoplasmic domain start (Cowan, et al., Immunogenetics, 25, 241-250 (1987)). Collectively, these finding underscore the exceptional sensitivity of scrHLA-typing in detecting clinically relevant coding variations withing HLA. Crucially, the current method allows detailed analysis of HLAvariation within specific cellular contexts, providing valuable insights with significant clinical implications for patients experiencing leukemia relapse post-transplant.

Discussion By combining targeted hybridization capture, PacBio HiFi sequencing and an iterative genotyping-quantification pipeline, scrHLA-typing accurately calls and counts alleles including when multiple donor/recipient haplotypes coexist at disproportionate levels—a context where current bulk and short-read methods fail.

In post-alloHCT pediatric AML samples, scrHLA-typing correctly predicted classical class I and II HLA genotypes concordant with clinically-available donor/recipient genotyping and orthogonal genetic demultiplexing. The assay retained its accuracy across extreme levels of chimerism and was able to quantify statistically significant cell type-specific ASE which were prevalent in malignant cells of some, but not all patients. This ability to discriminate meaningful ASE at single-cell resolution enables a new dimension of post-alloHCT monitoring with clinical implications for salvage therapy planning. Finally, the assay enabled high-fidelity detection of genetic variation within HLA exonic regions, though in this small cohort, no variants were identified that could be attributable to patients' relapse.

A major advantage of the present assay is its application of high-fidelity long-read sequencing at the single-cell level to resolve highly complex HLA loci—an advance over previous single-cell HLA studies (Darby, et al., Bioinformatics, 36, 3905-3906 (2020); Solomon, et al., Front. Immunol., 14 (2023); Kang, et al., Nat. Genet., 55, 2255-2268 (2023)), which, while conceptually elegant were confined to short-read sequencing and limited to 2-field HLA resolution. This is the first study to achieve in RNA three-field HLA genotyping and quantify alleles in complex chimeric mixtures such as those encountered in the post-alloHCT setting. The high base accuracy of the current approach facilitates detection of somatic mutations in HLA, which are known to recur in cancer (Shukla, et al., nat. Biotechnol., 33, 1152-1158 (2015)). Such mutations can result from errors during DNA repair or replication (Vijg & Dong, Cell, 182, 12-23 (2020); Caldecott, Trends Biochem. Sci., 49, 68-78 (2024); Chang, et al., Nat. Rev. Mol. Cell Biol., 18, 495-506 (2017); Zhao, et al., Nat. Rev. Mol. Cell Biol., 21, 765-781 (2020)), but to be detectable, should involve a relatively small number of nucleotides.

It is anticipated that scrHLA-typing could have immediate clinical utility in risk-stratifying relapsed alloHCT patients prior to salvage therapy, by identifying likely mechanism of immune escape and guiding therapeutic decisions. Critically, the assay can provide such insight even at low disease burden, when targeted interventions are most likely to succeed. Looking forward, assessing variability in HLA expression within leukemic cell clusters at diagnosis may also inform donor selection—prioritizing mismatches in alleles with consistently high and stable expression over those with lower or more variable expression—thereby optimizing graft-versus-leukemia potential from the outset.

Methods. Specimen collection and processing. Bone marrow aspirates (BMA) were obtained from the pelvic bone of patients and were processed using density gradient centrifugation to isolate mononuclear cells. Any remaining red blood cells (RBCs) were depleted by incubating the mononuclear cells in an RBC-lysis buffer (NH4CI 8.3 g/L; NaHCO3 1 g/L; 0.04 g/L EDTA; filtered 0.22 μm) for 10 min at room temperature, then washed in 1×PBS with 2% FBS. Cells could be viably cryopreserved in fetal bovine serum with 10% DMSO stored in liquid nitrogen for later thaw and use, or immediately used in the next processing steps. Cells were counted on a Nexcelom instrument to determine numbers and viability using ViaStain AOPI Staining (Nexcelom #CS2-0106-25 mL). When viability<80%, live cells were enriched using magnetic removal of dead cells (Miltenyi Biotec 130-090-101). When applicable, at most 10 million cells were enriched for cells with CD34 cell surface markers using either Miltenyi magnetic CD34 Microbead kit (Miltenyi Biotec #130-097-047) or EasySep human CD34 Positive Selection kit (Stemcell #17856) with an expected yield of 500,000 CD34-enriched cells. For patient ‘AML15’, instead of a CD34+ enrichment, cells with CD117 markers were enriched using Miltenyi magnetic CD117 Microbead kit (Miltenyi Biotec #130-091-332) in a decision based on the patient's leukemia characteristics.

Feature barcode cell staining. To profile protein expression at the single cell level, DNAbarcoded antibody staining was performed (Feature Barcodes). Compatible with the 10× Genomics Next GEM Single Cell 5′ v2 with Feature Barcode chemistry, the TotalSeq™-C Human Universal Cocktail V1.0 (BioLegend #399905) including 137 antibodies recognizing 130 unique cell surface/principal lineage antigens and 7 isotype controls (list of antibodies: https://www.biolegend.com/Files/Images/BioLegend/totalseq/TotalSeq_C_Human_Universal_Cocktail_vi_137_Antibodies_399905_Barcodes.xlsx) was used when applicable. The universal cocktail was designed for the multiomic characterization of peripheral blood-derived immune cells. For optimal characterization of BMA-derived cells, the antibodies CD15 (clone W6D3), CD34 (clone 581), and CD117 (clone 104D2) (BioLegend TotalSeq™-C chemistry #C0392, C0054, and C0061, respectively) were spiked in. Briefly, 1 million cells were resuspended into 20 μL 1×PBS with 2% FBS. Lyophilized TotalSeq™ panel was equilibrated at room temperature for 5 min, spun down at 10,000 g for 30 s, resuspended in 27.5 μL 1×PBS with 2% FBS, and spun down again at 20,000 g for 5 min at 4° C. To the cell suspension, 5 μL of Fc blocking reagent was added (FcX; BioLegend #422301) followed by 5 min incubation. 25 μL of the resuspended TotalSeq™ panel was added, then 1 μL of each of the spike-in antibodies were added for a final volume of 50 μL and incubated for 20 min at 4° C. The cells were then washed 3 times (400× g for 6 min) and counted for quality and quantity assessment using ViaStain AOPI Staining (Nexcelom #CS2-0106-25 mL).

Single cell RNA and Feature Barcode capture. While the focus was on HLA pulldown from cDNA pools barcoded using 10× Genomics 5′ chemistry, the pulldowns and generated data from cDNA captured using 10× Genomics 3′ chemistry were also collected, in order to compare both capturing chemistries. Therefore, single cell whole RNA capture and DNA-barcoded cell surface proteins capture (when applicable) were performed using either the 10× Genomics Next GEM Single Cell 3′ v3.1 with Feature Barcode chemistry or the 10× Genomics Next GEM Single Cell 5′ v2 with Feature Barcode chemistry. For the generation of cell-barcoded complimentary DNA (cDNA) molecules, the appropriate 10× Genomics User Guide were followed (document #CG000317_Rev_B for the 3′ chemistry; #CG000330_Rev_A for the 5′ chemistry). Briefly, cells were prepared for capture in 1×PBS with 2% FBS at 1,000 cells/uL. 10,000 cells were submitted for capture using the 10× Chromium Controller. Following reverse transcription and cell barcoding in droplets, emulsions were broken and cDNA was purified using Dynal MyOne SILANE (ThermoFisher Scientific #37002D) followed by a PCR amplification (3′ v3.1 chemistry: 98° C. for 3 min; 12 cycles of 98° C. for 15 s, 63° C. for 20 s, and 72° C. for 1 min; 5′v2 chemistry: 98° C. for 45 s; 13 cycles of 98° C. for 20 s, 63° C. for 30 s, and 72° C. for 1 min). Size selection was used to separate the amplified cDNA molecules (>800 bp) and the DNA from cell surface protein Feature Barcode (<800 bp) for separate downstream applications, using SPRIselect reagent (Beckman Coulter #B23318). The majority of cDNA molecules in the final product had expected sizes (600-2000 bp).

Cell surface protein library construction and sequencing. Amplified DNA from cell surface protein Feature Barcodes was used for library construction. Illumina adaptors and TruSeq/Nextera sample indexes (P5, P7, i7 and i5) were added via PCR (98° C. for 45 s; 5-7 cycles of 98° C. for 20s, 54° C. for 30 s, and 72° C. for 20 sec). Amplified DNA libraries were size selected and purified with an expected Agilent Bioanalyzer trace between 200-250 bp. Libraries were sequenced on an Illumina NextSeq2000 sequencer using a pair-ended dual indexing configuration with 28 bp (Read1), 10 bp (i7 Index), 10 bp (i5 Index), 90 bp (Read2) for3′ capture chemistry, and 26 bp (Read1), 10 bp (i7 Index), 10 bp (i5 Index), 90 bp (Read2) for 5′ capture chemistry, for a sequencing depth of >5,000 read pairs per cell.

Gene expression library construction and sequencing. For gene expression library construction, 50 ng (5′ capture chemistry) or between 25-150 ng (3′ capture chemistry) of amplified cDNA were enzymatically fragmented, end-repaired, A-tailed, and ligated to a partial TruSeq adaptor. Additional Illumina adaptors and TruSeq/TruSeq sample indexes (P5, P7, i7 and i5) were added via PCR (98° C. for 45 s; 14 cycles of 98° C. for 20 s, 54° C. for 30 s, and 72° C. for 20 sec). Amplified DNA was size selected and purified with an expected size range between 300-600 bp. Libraries were sequenced on an Illumina NextSeq2000 sequencer using a pair-ended dual indexing configuration with 28 bp (Read1), 10 bp (i7 Index), 10 bp (i5 Index), 90 bp (Read2) for 3′ capture chemistry, and 26 bp (Read1), 10 bp (i7 Index), 10 bp (i5 Index), 90 bp (Read2) for 5′ capture chemistry, for a sequencing depth of >20,000 read pairs per cell.

Targeted hybridization and magnetic capture of HLA from single cell-barcoded whole transcriptome and long-read sequencing. The scrHLA-typing workflow was designed for in-depth targeted sequencing of HLA genes from a single-cell barcoded whole transcriptome. The 5′-biotinylated hybridization probes were designed to recognize almost all known classical class I and II and non-classical (including pseudogenes) HLA alleles belonging to 40 genes in the IMGT/HLA database3, specifically: HLA-A, -B, -C, -E, -F, -G, -H, -J, -K, -L, -T, -V, -W, -Y, -DMA, -DMB, -DOA, -DOB, -DPA1, -DPA2, -DPB1, -DPB2, -DQA1, -DQA2, -DQB1, -DRA, -DRB1, -DRB2, -DRB3, -DRB4, -DRB5, -DRB6, -DRB7, -DRB8, -DRB9, as well as HFE, MICA, MICB, TAP1, and TAP2. The workflow can be adapted to any platform capturing single cells and barcoding their transcriptome, including 10× Genomics as described here. The starting material is 10× Genomics-generated full-length cDNA, leftover from what is used to create the Illumina indexed libraries in downstream steps.

Optimal reamplification. To capture the gene transcripts encoding the HLA molecules, an enrichment was performed using xGen™ Hybridization and Wash v2 kit reagents (IDT #10010351), with modifications. Briefly, 500 ng of barcoded cDNA was needed to start. When not enough cDNA mass was available (<500 ng), additional cDNA mass was generated by mixing leftover cDNA with KAPA HiFi 2× ReadyMix (Roche #KK2602) and both primers from the 10× cDNA amplification step (10× Genomics #PN-2000089; Table 3) at final concentration of 300 nM each, followed by a PCR (98° C. for 45 s; 3-to-5 cycles of 98° C. for 20 s, 62° C. for 20 s, and 72° C. for 3 min) and a SPRIselect cleanup (0.6× bead-to-DNA solution ratio).

Hybridization and magnetic pulldown. A volume containing 500 ng of cDNA was mixed with 7.5 μL of human Cot1-DNA (supplied in the xGen™ kit) and concentrated on SPRIselect beads using a 1.8× ratio of SPRIselect bead-to-DNA solution suspension. The cDNA was eluted into 19 μL of hybridization mix. In addition to the 2× Hybridization Buffer and the 6.3× Hybridization Enhancer (supplied in the xGen™ kit), the hybridization mix included two blocking oligonucleotides customdesigned to cover the regions at both ends of one of the two strands of a captured cDNA molecule, to block unwanted amplification artifacts. Specifically, where the constant Illumina TruSeq Read1, TSO, and Poly(dT) sequences were located, but also where the highly variable cell barcode and/or unique molecular identifier (CB:UMI) were located. The oligos were modified at their 3′ end by adding a C3 phosphoramidite spacer to prevent them from extending (i.e., acting as primers) in subsequent PCRs (Table 2). The blocking oligos' final concertation in the hybridization mix was 1.6 μM. A mix of biotinylated 97-base long probes designed to be highly specific of the HLA transcripts, was also added to the hybridization mix (Table 1). The probe mixture final concentration in the hybridization mix was 189.5 nM. The hybridization mix was heated at 95° C. for 30 s followed by a rapid ramp down to 65° C. which was held for 4 hours allowing the biotinylated probes to bind to the target cDNA. Meanwhile, wash buffers (WB) I, II, and Ill, and the stringent wash buffer (supplied in the xGen™ kit) were prepared from 10× concentrates with enough volume for two 100 μL washes with WB I, two 100 μL washes with the stringent WB, and one 100 μL wash with each of WB II and Ill. Streptavidin beads were used to capture the probes bound to their targets. 50 μL of Dynabeads M-270 Streptavidin (ThermoFisher Scientific #65305) were washed two times in a Beads Wash buffer (prepared from the 2× concentrate supplied in the xGen™ kit) and resuspended in the hybridization mix at the end of the 4-hour incubation, immediately followed by an incubation at 65° C. for an additional 45 min with frequent gentle hand mixing every 10-15 min. At the end of the incubation, the beads were washed on a magnetic rack one time with WB I pre-heated at 65° C. and two times with stringent WB pre-heated at 65° C. followed by a series of one-time washes with WB I, II, and Ill, at room temperature. At the end of the last wash, the beads were removed from the magnet and resuspended in EB buffer (Qiagen #19086).

Amplification of hybridization product. Following hybridization capture of HLA molecules, an on-bead PCR was performed to amplify captured molecules. The product (beads+captured cDNA) was mixed with KAPA HiFi 2× ReadyMix (Roche #KK2602) and both primers from the 10× cDNA amplification step (10× Genomics #PN-2000089; Table 3) at final concentration of 300 nM each, followed by a PCR (98° C. for 45 s; 10-14 cycles of 98° C. for 20 s, 62° C. for 20 s, and 72° C. for 3 min), and a SPRIselect cleanup (0.8× bead-to-DNA solution ratio).

TSO artifact removal. A significant fraction of the cDNA product from a 10× Genomics single cell cDNA preparation (3′ or 5′ chemistry) may contain a template switch oligo (TSO) priming artifact (i.e., TSO sequence at both ends of the cDNA molecule) instead of the correct structure. TSO artifact depletion is recommended for long-read sequencing on platforms such as Pacific Biosciences. Following hybridization capture and PCR-amplification of the pulldown product, TSO artifact depletion was performed (AL'Khafaji, et al., Nat. Biotechnol., 42, 582-586 (2024)) by first mixing the amplified pulldown product with KAPA HIFI 2× Uracil+(Roche #KK2802), a biotinylated forward primer with two uracil additions at the 5′ end, and a reverse primer. Final primer concentration was 500 nM. The biotinylated uracil primer captured the constant region on the opposite end of where the CB:UMI and TSO sequences are located on the cDNA molecule while the regular primer captured the constant region that encompassed the CB:UMI and TSO sequences (Table 3). The mix was amplified by PCR (98° C. for 45 s; 4 [if cDNA input 250 ng] to 6 [if cDNA input 25 ng]cycles of 98° C. for 20 s, 65° C. for 30 s, and 72° C. for 4 min) followed by a SPRIselect cleanup (0.8× bead-to-DNA solution ratio). cDNA molecules with the correct structure were then captured and purified using 10 μL (100 ug) Dynabeads™ kilobaseBINDER™ streptavidin beads (ThermoFisher #60101) in 40 μL of provided Binding Solution, incubated 15 min at room temperature, washed on magnet 2 times by provided Washing Solution and 1 time by distilled water, and finally reconstituted in 40 μL EB. After streptavidin binding, 2 μL of USER enzyme (NEB #M5505L) was added to the bead/cDNA mixture and incubated at 37° C. for 30 min to uncouple the cDNA from the beads. After magnetic separation, the supernatant containing the cDNA was purified by a SPRIselect cleanup (0.8× bead-to-DNA solution ratio) and quantified.

TSO-depleted reamplification for blunt-end product. The sample was reamplified after TSO depletion, to get back regular blunt ends on the product, by mixing it with KAPA HiFi 2× ReadyMix (Roche #KK2602) and both primers from the 10× cDNA amplification step (10× Genomics #PN-2000089; Table 3) at final concentration of 300 nM each, followed by a PCR (98° C. for 45s; 4 cycles [if 125 ng input cDNA], 3 cycles [if 125-500 ng input cDNA], or 2 cycles [if 500 ng input cDNA] of 98° C. for 20 s, 62° C. for 20 s, and 72° C. for 3 min) and a SPRIselect cleanup (0.8× bead-to-DNA solution ratio).

Constructing PacBio SMRTbells. The standard long-read sequencing approach was to multiplex up to 4 hybridization capture products on a PacBio ‘single molecule real-time’ (SMRT) Cell 8M, using the ‘Iso-Seq’ method. Following TSO-depletion of the pulldown captured cDNA, the product was DNA-repaired, A-tailed, SMRTbell overhang adaptor-ligated (each sample with one of the barcoded Overhang Adaptors 8A/8B [PacBio #101-628-400/500] when multiplexing samples on a single SMRT Cell), nuclease-treated, and SMRTbell-cleanup purified (1× bead-to-DNA solution ratio) using the PacBio SMRTbell Prep kit 3.0 (PacBio #102-141-700), as per manufacturer's protocol (PacBio Procedure & Checklist #102-359-000 REV03 DEC2023). Libraries were sequenced on a PacBio Sequel Ile with 2-hour pre-extension time and 30-hour movie time.

Augmenting long-read throughput by multiplexed arrays isoform sequencing (MAS-seq). When augmenting throughput by MAS-seq was desired, the following intervention to the hybridization pulldown workflow was performed. Following TSO-depletion and right before the blunt-end reamplification, the TSO-depleted product was brought through the MAS-PCR step of the PacBio Procedure & Checklist for MAS-seq library prep (PacBio #102-678-600 REV03 MAR2023), inspired by Al'Kafaji et al.'s design AL'Khafaji, et al., Nat. Biotechnol., 42, 582-586 (2024)). Briefly, after TSO depletion, 50 ng of the TSOdepleted product in 45 μL of buffer was mixed with 125 μL of nuclease-free water and 212.5 μL of MAS-PCR mix (PacBio #102-692-800) or KAPA HIFI 2× Uracil+(Roche #KK2802), was split into 16 tubes (22.5 μL per tube) to each of which 2.5 μL of MAS primers premixes ‘A’ through ‘P’ (provided in PacBio's MAS-seq kit #102-659-600) was added, and PCR-amplified (98° C. for 3 min; 9 cycles of 98° C. for 20 s, 68° C. for 30 s, and 72° C. for 4 min). Products from the 16 reactions were pooled and cleaned up using either SMRTbell cleanup beads (PacBio #102-158-300) or ProNex Size Selection Chemistry beads (Promega #NG103B) using a 1.5× bead-to-DNA solution ratio. The rest of the USER enzyme (NEB #M5505L) digestion, ligation of pooled product into arrays sealed on both their ends with MAS adaptors, DNA damage repair and nuclease treatment of the arrays, was conducted according to manufacturer's protocol (PacBio #102-678-600 REV03 MAR2023). Arrays were sequenced on a PacBio Sequel lie with 2-hour pre-extension time and 30-hour movie time.

Targeted hybridization and magnetic capture of specific mutated transcripts from single cell-barcoded whole transcriptome and long-read sequencing for gene-fusion detection or short-read sequencing for SNV detection. A high adaptable workflow for targeted hybridization capture and amplification of gene transcripts with known mutations or fusions from the whole amplified transcript pool (cDNA) was designed. For patients ‘AML1’, ‘AML4’ and ‘AML6’, mutations of the single nucleotide variants (SNVs) type and/or gene fusions were identified elsewhere, through clinically validated commercial assays. Once identified, 5′-biotinylated hybridization oligonucleotide probes appropriately specific to the gene(s) carrying the mutation(s) were designed (Table 4). If destined for short-read sequencing, reverse PCR-primer oligonucleotide(s) positioned a few nucleotides downstream each SNV were designed. The fusion-specific and long-read sequencing experimental protocol was the same as for HLA targeted capture (see above) except that the biotinylated capture probes added to the hybridization mix were specific to the fusion breakpoints (FIG. 21A).

The SNV-specific and short-read sequencing experimental protocol followed the same first steps as for the HLA targeted capture (see above), with a starting material at 500 ng of cell-barcoded full-length cDNA (here, generated by the 10× Genomics platform), and the ‘optional reamplification’ and ‘hybridization and magnetic pulldown’ steps. Following hybridization capture, an on-bead targeted PCR was performed to amplify regions that included the mutations of interest. The forward primer universally recognized 10× Genomics-generated cDNA molecules and started at the CB:UMI location, while the reverse primer(s) were designed to specifically bind to a region 40-70 bp downstream the mutation on the captured transcripts. The PCR mix consisted of the KAPA HiFi 2× ReadyMix (Roche #KK2602), the captured cDNA molecules on their beads, a universal 10× Genomics compatible forward primer, and as many custom-designed reverse primers as there were mutations to resolve. Each primer was at a final concentration of 500 nM. A PCR was then carried out (98° C. for 45 s; 14-18 cycles of 98° C. for 20 s, 62° C. for 20, and 72° C. for 3 min), followed by a SPRIselect cleanup step while carefully choosing a size selection ratio compatible with the expected size of the smallest amplicon in the PCR product. As a quality control measure, the product size was assessed and the number of peaks in the trace profile corresponded to the number of expected amplicons in the PCR product.

Following targeted PCR, the amplicons were end-repaired, A-tailed, and adaptor-ligated using the NEBNext® Ultra™ II End Repair/dA-Tailing and Ligation Module reagents (NEB #E7546S/L and #E7595S/L). 50 μL of captured and target-amplified cDNA was mixed with 7 μL of End Prep buffer and 3 μL of End Prep enzyme, and incubated 30 min at 20° C. then 30 min at 65° C. After incubation, 30 μL of Ligation Mix, 1 μL of Ligation Enhancer, and 7 μL of 10× Adaptor Oligo (10× Genomics #PN-2000094) were added to the previous mix and incubated 20 min at 20° C. A SPRIselect cleanup followed, and the eluted product underwent an indexing PCR (98° C. for 45 s; 7 cycles of 98° C. for 20 s, 54° C. for 30 s, and 72° C. for 3 min) to add Illumina compatible TruSeq/TruSeq P5, P7, i7, and i5 adaptors using the 10× Genomics Dual Index Plate TT Set A (10× Genomics #PN-3000431) mixed with the KAPA HiFi 2× ReadyMix. Libraries were sequenced on an Illumina NextSeq2000 sequencer using a pair-ended dual indexing configuration with 26 bp (Read1), 10 bp (i7 Index), 10 bp (i5 Index), 90 bp (Read2), for a sequencing depth between 500-2,000 read pairs per cell.

Single cell gene expression and cell surface protein data processing with Seurat. Illumina BCL files were demultiplexed and converted to FASTQ format using ‘bcl2fastq’ version 2.20.0 (Illumina, Inc.) through the cellranger-mkfastq function (10× Genomics, Inc.). Resulting FASTQ files were processed with ‘cellranger’ (version 6.0.2) to identify and count Feature Barcodes, and to align to the hg38 reference genome and count Gene Expression data. An output filtered feature barcode matrix file was then read on R (Edfors, et al., Mol. Syst. Biol., 12, 883 (2016)) (version 4.2.0) and converted into a ‘Seurat’ object (Hao, et al., Cell, 184, 3573-3587.e29 (2021)) (version 5.0.1). Initial quality control was used to subset out cells with too many (>50,000) or too few (<1,000) RNA counts and too many mitochondrial RNA counts (>20% of a cell's total RNA counts). Counts were then normalized assuming proportionality to the sum of counts of every feature per CB (‘LogNormalize’ method, scale factor: 10,000), variable features were selected (“vst” selection method), features were centered and scaled (“ScaleDatao” function), principal component analysis on the variable features was run, and UMAP low dimensional embedding (Becht, et al., Nat. Biotechnol., 37, 38-44 (2019)) and clustering using k-nearest neighbor algorithm was performed on data.

Automatic cell type labeling using viewmastR. The cell types were automatically labeled usingthe ‘viewmastR’ package (Krakow, et al., Blood, 144, 1069-1082 (2024); Furlan, Automated single-cell genomic cell type assignment) (version 0.2.1), a machine-learning platform performing unsupervised classification of single cells between the query dataset and a well-annotated reference dataset using multinomial logistic regression, relying on the top 5,000 genes commonly variable across the datasets. In the present study of BMMCs from bone marrow aspirates, previously annotated reference dataset of healthy marrow and peripheral blood mononuclear cells (Granja, et al., Nat. Biotechnol., 37, 1458-1465 (2019)) were used.

Genetic demultiplexing of allogeneic entities with soupourcell. To perform variant calling and distinguish donor vs. recipient cells of bone marrow samples post-alloHCT, first the raw sequence data aligned to hg38 files (the BAM files) output from cellranger were merged using ‘mergebams’ (version 0.3), a custom script that preserves sample tags onto CBs (Furlan-Lab/Mergebams (2022)). Then, ‘souporcell’ (Heaton, et al., Nat. Methods, 17, 615-620 (2020)) (version>2.0; ‘singularity’ version 3.5.3) was used on the merged BAM files as input, the cell-ranger output sample filtered cell barcodes as the barcode file, the hg38 reference sequence as the reference file, a variants file (VCF format) from the 1000Genomes project as the common variants file, an integer number “k” as the number of expected genotypes in the sample, and invoking the “ignore” and “skip_remap” options. Sparse mixture model output from souporcell was log-normalized and colored by the genotype assignment.

IsoSeq computational processing of long-read HLA pulldown sequencing data. Error-corrected sequencing reads were generated by the Circular Consensus Sequencing (CCS) workflow, on-board the PacBio Sequel lIle instrument (Pacific Biosciences of California, Inc.), generating a high fidelity “HiFi” reads BAM file. When HiFi reads were concatenated (i.e., from MAS-seq), an additional segmentation step was performed by providing the list of MAS adaptors and running the ‘split’ program in the PacBio concatenated read splitter ‘Skera’ package. HiFi or segmented HiFi reads were then processed in the ‘IsoSeq’ suite of computational tools (version 3.8.2) by following those programs in order: 1) ‘lima’ (version 2.7.1) was used to remove sequencing primers, CTACACGACGCTCTTCCGATCT (SEQ ID NO: 71) (5p) and GTACTCTGCGTTGATACCACTGCTT (3p) for the 10×5′ chemistry reads, and AAGCAGTGGTATCAACGCAGAGTACATGGG (SEQ ID NO: 70) (5p) and AGATCGGAAGAGCGTCGTGTAG 3p) for the 10×3′ chemistry reads, 2) ‘tag’ was used to separate the UMI and CB from the rest of the transcript using the ‘16B-10U-T’design (10×5′ chemistry) or the ‘T-12U-16B’ design (10×3′ chemistry), 3) ‘refine’ was used to trim the polyA sequence and remove unintended concatemers (if not already done by Skera), and 4) ‘correct’ was used to identify cell barcode errors and correct them against the 10×5′ chemistry CB whitelist or the 10×3′ chemistry reverse complemented CB whitelist. All of the IsoSeq pipeline steps were used with default settings.

scrHLAtag to align long-read HLA pulldown sequencing data to the IMGT/HLA reference. This experiment involved developing ‘scrHLAtag’ (cersion 0.1.7), which is a command line tool written in Rust for processing full-length single cell-barcoded HLA transcript-enriched PacBio long-read sequencing data from 10z Genomics. scrHLAtag was designed to use ‘minimap2’ (version 2.24), primarily designed for fast and high-accurate alignment of long-reads (Li, Bioinformatics, 24, 3094-3100 (2018)), to align sequences to the mRNA/cDNA reference library (exons only) and to the genomic reference library (exons, introns and/or UTRs) of all known HLA alleles available from the IMGT/HLA database (Barker, et al., Nucleic Acids Res., 51, D1053-D1060 (2023)) (version: 3.60.0-2025-04).

Because scrHLA-typing is based on single cell RNA-seq data, HLA alleles differing in non-coding regions cannot be resolved by the present assay. Therefore, only up to 3-field HLA allele names were used. HLA alleles which only varied in the 4th field from each other (e.g., A*30:02:01:01, A*30:02:01:02, A*30:02:01:03, etc.) were reduced to one 3-field allele in the references, where the sequence of only the corresponding XX:XX:XX:01 allele was retained. The 42,579 cDNA alleles and the 24,355 genomic alleles known from IMGT/HLA version 3.60.0 became 31,141 and 16,916 alleles (respectively) after reduction to 3 fields.

The IsoSeq “corrected” BAM files were used as input to scrHLAtag. The output of scrHLAtag included BAM files aligned to the IMGT/HLA transcriptome and/or genome indexed and sorted by read name (‘SAMtools’; version 1.16.1), file(s) summarizing read counts per CB:UMI, FASTA references used by minimap2, and molecule information files with each line representing one read count and containing the sequencing read with its associated CB and UMI, the allele to which it was mapped, and other minimap2 alignment metrics.

Running unsupervised (i.e., without providing a predetermined list of HLA alleles), scrHLAtag aligns the “corrected” BAM to the entire IMGT/HLA reference library. This typically yields count files with a few thousands unique HLA alleles mapped to the reference (between 4853 and 7828 unique alleles in the present study; FIG. 24), a number considered too high given that the target genes are 40, but not surprising given the high level of homology between the tens of thousands of alleles. scrHLAtag can also run “supervised” or “guided” if provided with a short list of HLA alleles. This list typically contains names of the top HLA candidates progressively refined via scrHLAmatrix (see below) after multiple scrHLAtag-scrHLAmatrix iterations using the same “corrected” BAM alongside the newly refined list as input. The convergence of the HLA candidate list to the putative HLA genotypes of each allogeneic entity in a given sample is known when such list stops updating after additional iterations.

scrHLAmatrix to predict HLA genotypes, perform deduplication, and summarize counts from scrHLAtag output. To analyze scrHLAtag output, ‘scrHLAmatrix’ (version 0.18.1) was developed, which is a package written in R to process scrHLAtag molecule information read count files to predict HLA genotypes in single cells, remove PCR duplicates, and summarize counts into Seurat-compatible (Hao, et al., Cell, 184, 3573-3587.e29 (2021)) matrices (FIG. 5B).

Filtering by read quality. Best quality alignment reads in molecule information count files were identified and retained. scrHLAtag collected from minimap2 a combination of metrics including the “s1” (chaining score), “AS” (dynamic programming alignment score), “NM” (total number of mismatches and gaps in the alignment), and “de” (gap-compressed per-base sequence divergence) tags. Assuming imperfections during alignment, it was hypothesized that the scores in these tags would be unfavorable if a query sequence was mapped to the wrong reference sequence. The availability of ‘Donor’ vs. ‘Recipient’ labeling of sequences by souporcell was exploited as ‘ground truth’ to compare the score values in correct vs. wrong mappings. Score thresholds of good quality mappings were set where the sum of True Positive Rates (donor or recipient mappings correctly validated by souporcell) and True Negative Rates (donor or recipient mismappings as predicted by scores, correctly refuted by souporcell) was maximized at each HLA locus, for each of the tags (FIG. 8). Across the loci and their alleles, on average, thresholds for “s1” and “AS” were such that good quality mappings had a percentage passing score of 81.8 and 80.7, respectively, from the maximum scores of each locus. For “NM” and “de”, good quality mappings had scores at or below 21 and 0.004, respectively, on average across the loci (FIG. 8). Thus, default “s1” and “AS” percent passing scores were set, and default “NM” and “de” thresholds in the scrHLAmatrix functions at 80, 80, 15 (slightly more stringent), and 0.01 (slightly less stringent), respectively.

Classifying cells based on HLA genotype patterns. With the presence of chimeric entities within the captured single cells, it was hypothesized that the distribution of identified HLA alleles among the cells would reveal patterns, mostly driven by the recipient-donor mismatched alleles. To visualize these patterns, molecule information files were first organized into “HLA'CB” count matrices. With the option to match the CBs of the count matrix with those of the associated Seurat object (allowing the analysis of previously quality controlled CBs), the counts of each allele were normalized assuming proportionality to the sum of counts of every HLA allele per CB (normalization by size factor) and log-transformed. Next, a principal component analysis (PCA) was performed to emphasize the patterns. By default, up to the top 50 PCs were retained for downstream clustering and UMAP (uniform manifold approximation and projection) analyses. Increasing or decreasing this number according to the scree plot or “elbow” plot shape, was optional to retain the top PCs accounting for most of the variation and discard the bottom “noisy” PCs. To classify cells into groups with common HLA genotype expression patterns, a graph-based clustering approach was applied. Algorithm choices included a community structure detection method: the Leiden algorithm (Tragg & van Eck, Sci. Rep., 9, 5233 (2019)) (‘leiden’: version 0.4.3), a density-based method: DBSCAN (Ester, et al., Proceedings of the Second International Conference on Knowledge Discovery and Data Mining, 226-231 (1996)) (‘dbscan’: version 1.1.10), a connectivity-based method: hierarchical clustering (agglomerative), a centroid-based method: k-means (both “hclust” and “kmeans” provided by the ‘stats’ package part of R54, version 4.2.0), and a distribution-based method: Gaussian Mixture Model (provided by the ‘mclust’ package (Scrucca, et al., R J., 8, 289-317 (2016)), version 6.0.0). All clustering algorithms were applied to PCA space, except DBSCAN, known for its degraded performance on multidimensional data (Bhattacharjee & Mitra, Front. Comput. Sci., 15, 151308 (2020)). Instead, non-linear manifold-aware dimension reduction was performed of the retained PCs with UMAP (McInnes & Melville, UMAP: Uniform Manifold Approximation and Projection for Dimension Reduction, (2020)) (‘uwot’: version 0.2.2) down to 2 dimensions, onto which ‘dbscan’ was applied.

The UMAP parameters were further tuned to generate denser clumps and cleaner separations between clusters for better DBSCAN performance—specifically, setting “min_dist” and “repulsion_strength” to the low values of 0.001. The number of genotype classes could be fixed a priori and in this study was equal to 2 (“k=2”) representing the donor and recipient entities in the samples, although that number could be greater than 2 (see below regarding in-silico mixing≥3 chimeric entities). The assumptions on which algorithm had the best ability to correctly classify cells into the different chimeric entities, was not set. Instead, clustering “consensus” was extracted by grouping the CBs into the same cluster if they agreed on membership in a majority of clustering algorithms clusters (low confidence classifications were set as “not assigned”). The consensus algorithm's performance in correctly predicting the chimeric entities depended on the number of donor-vs.-recipient mismatched alleles among total alleles, with greater proportion of mismatches leading to classification accuracy close to 100% (FIG. 24; and FIG. 17 to see examples of HLA pattern-based classification vs. souporcell classification in a patient with 3 samples at various levels of chimerism).

Predicting HLA genotypes. A “per-single-cell” prediction algorithm was performed, consisting first of summarizing counts into “HLA×CB” matrices for each identified cluster of cells, separately. Then, an account of the top 2 most numerous alleles per loci and per CB was performed. The “top 2 alleles” was based on the assumption that a cell would normally have at most 2 alleles per locus. Another assumption was that correctly aligned alleles would be more abundant than misaligned or slightly misaligned alleles, thus more likely to be retained in a per-cell tally. Ties where ≥3 alleles were equally abundant or where ≥2 alleles were equally abundant at the 2nd place, were resolved by eliminating tied alleles due to undecidability. The resulting genotypes per loci per CB, either apparent heterozygous (i.e., “Allele1_Allele2”) or apparent homozygous (i.e., “Allele1_blank”) were then ranked from most to least abundant for each chimeric cluster. The highest-ranking genotypes cumulatively accounting for a majority of total counts for a locus (by default 85% of the total) were retained in the final list of candidate alleles. When scrHLAtag runs returned several thousand uniquely mapped alleles (most commonly during the 1st “unsupervised” runs), the “HLA×CB” count matrices would become too sparse, and the per-single-cell prediction algorithm would no longer yield accurate genotype predictions. By default, when the number of unique HLA alleles was >3000, a simpler “pseudo-bulk” prediction algorithm was used. The algorithm handled the molecule information count files as bulk data frames. For each identified cluster of cells, the highest-ranking mapped alleles in terms of bulk counts, cumulatively accounting for a majority of total counts for a locus (by default 85% of the total) were retained in the final list of candidate alleles. The list of candidate alleles was then ready to be supplied to the next run of scrHLAtag.

Note that among the 31,141 reference entries of the mRNA/cDNA 3-field reduced IMGT/HLA library, 6 of them had an extra 24 to 99 validated nucleotides downstream the 3′ furthest most common sequencing starting position in the reference library. The alleles were: DPA1*02:38Q, A*03:437Q, B*13:123Q, C*02:205Q, C*04:09L (previously named C*04:09N), and C*04:61N, and considered rare in the populations. When the PacBio long-read RNA sequences—often longer than typically available reference sequence length—of the more common alleles DPA1*02:02:02, A*03:01:01, B*13:02:01, C*02:02:02, C*04:01:01, and again C*04:01:01 were aligned to the reference library, minimap2 of scrHLAtag preferentially matched them, respectively, to those erroneous references, despite incurring a 1 to 2 nucleotide(s) mismatch penalty (note: between C*04:09L and C*04:61N, a C*04:01:01 sequence would always preferentially misalign to the former, because it would incur fewer penalties vs. the latter). To mitigate this, an option was made available to adjust the final list of candidate alleles accounting for those 6 known misalignments.

Post-alignment PCR-deduplication. Algorithms to remove PCR duplicates based on a “consensus quality value” guided approach to generate one consensus sequence per founder (such as the “Dedup” program in IsoSeq) were avoided. Because mismatches and shifts “naturally” occur in HLA, it is suspected that these types of deduplication might create consensus amalgams not fully aligning to any allele, thus not appropriate for HLA sequencing where the difference between alleles is sometimes a single nucleotide mismatch. Moreover, because of the high degree of nucleotide homology between alleles of the same HLA gene or different genes but of the same HLA class, the template switching PCR artifact (i.e., molecular swap, noted in some high quality read clusters with the same CB:UMI) was prevalent. To account for this and also to avoid ‘chimeric’ consensuses, an enumeration-based, post-alignment (with minimap2 of scrHLAtag) deduplication strategy was adopted, where among reads belonging to the same CB:UMI, the allele with the most counts prevailed. When ties were present with the most numerous aligned allele, the entire CB:UMI was dropped due to undecidability.

Summarizing counts in single cells in Seurat-compatible matrices. After running scrHLAtag for the final time (i.e., with the most curated list of candidate alleles), reads were filtered by minimap2 quality read tags (see above) and PCR-deduplicated (see above). Additional options for read filtering were available (FIG. 18A). These included resolving donor-vs.-recipient genotype conflict (assuming a cell cannot have both recipient and donor-origin HLA allele) by keeping either donor-specific or recipient specific HLA-associated UMIs (when known), based on their count difference per cell. Another option was resolving per-HLA genotype conflicts, assuming each cell had a maximum of 2 genotypes per HLA gene and keeping those with the most counts. A third option was correcting linkage disequilibrium (LD) in the HLA-DR subregion, assuming well established strong LD in the DR1 haplotype (DRB1*01 and *10 in LD with DRB6 and DRB9), the DR51 haplotype (DRB1*15 and *16 in LD with DRB6, DRB5, and DRB9), the DR52 haplotype (DRB1*03, *11, *12, *13, and *14 in LD with DRB2, DRB3, and DRB9), the DR8 haplotype (DRB1*08 in LD with DRB9), and the DR53 haplotype (DRB1*04, *07, and *09 in LD with DRB7, DRB8, DRB4, and DRB9), and removing reads associated with genes that are in conflict with the LD rules of the abovementioned DRB1 allele families. In practice, these additional options allowed the fine-graining of the reads count files without dramatically changing the counts (FIG. 18C). Finally, the reads were summarized in “HLA'CB” sparce numeric matrices.

Assessing mismatch patterns in HLA sequencing reads across single cells. Bioconductor tools were used to summarize and visualize mismatch patterns in HLA sequencing reads across single cells. The 3-field reduced HLA mRNA reference from scrHLAtag was forged into a ‘BSgenome’ class object (Kanaan, BSgenome.Hsapiens, (2025)). Then, the long-read HLA-aligned BAM files from each CD34-sorted and unsorted bone marrow specimens of each sample were first merged then subset into distinct groups of interest by CB (i.e., healthy donor, healthy recipient, or malignant recipient cells) using ‘mergbamsR’ version 0.0.5, and R package that was designed for the purpose (Furlan, mergebamsR: margining bams with R. R package version 0.0.5 (2024)). Finally, using ‘Rsamtools’ version 2.14.0 from Bioconductor (Morgan & Hayden, Rsamtools: Binary alignment (BAM) version 2.14.0, (2022)), alignment statistics were extracted and summarized.

In-silico mixes with 3 or more chimeric entities to test the HLA genotype-based cell classifier. In-silico mixes were necessary because samples naturally consisting of 3 or more chimeric entities were not available in this study (the closest clinically available candidates would have been a sample from a patient post-double cord blood transplant or a from a patient with a second or third transplant; however, even in those cases, one donor usually prevails69). CBs from the Seurat objects of AML4_d90, AML8, AML6, and AML1 were extracted based on whether their labels were “Donor” or “Recipient” (by souporcell). The “corrected” BAM files (output of PacBio's IsoSeq Correct program) were subset using SAMtools to only retain the reads associated the extracted CBs. Using mergebams, mixtures of 3, 4, 5, and 6 chimeric entities were created, making sure to tag sample labels to their corresponding CBs. Post-merge BAMs were then input to scrHLAtag, in an unsupervised way initially, and subsequently into multiple rounds of scrHLAtag guided by a gradually converging list of HLA candidates predicted by scrHLAmatrix. Given a value “k” of “3”, “4”, “5”, or “6” respectively corresponding to each of the 4 in-silico mixes, the scrHLAmatrix classifier grouped cells into 3, 4, 5, or 6 clusters based on HLA expression patterns. The classification accuracy, calculated as the percentage of cells within a given classification corresponding to the same majority label (known a priori), ranged from 98.2% to 100% (FIG. 12A; FIG. 12B).

Mutational short-read computational data processing. Illumina BCL files were demultiplexed and converted to FASTQ format using bcl2fastq through the cellranger-mkfastq function, as the mutational targeted sequencing and hybridization capture workflow was adapted to the 10× Genomics workflow. Resulting FASTQ files were then processed with ‘mutCaller’ (version 0.7.0), a command line tool written in Rust was developed to call mutations in sequencing data70. To align sequences to the hg38 reference genome, mutCaller used minimap2 by default when provided with unaligned FASTQs. The other option was ‘STAR’ (Dobin, et al., Bioinformatics, 29, 15-21 (2013)) (version 2.7.2a). mütCaller then counted CB:UMIs with reference nucleotides and with alternative nucleotides in the alignment BAM files based on a list of a priori reference vs. alternative (or query) nucleotides at a given chromosomal location. A given UMI for a given CB was considered “reference” genotype if reference counts for the CB+UMI combination was greater than alternative counts, and vice versa. Then “reference” and/or “alternative” UMIs for a given CB were summarized and such CB was called as expressing the “reference” (i.e., wildtype) transcript, the “alternative” (i.e., mutant) transcript, or both (i.e., biallelic).

Fusion-gene long-read computational data processing. First, the generation of “HiFi” reads BAM files from CCS reads was performed. Then the following IsoSeq pipeline steps (with default settings) were performed in the following order: ‘lima’, ‘tag’, ‘refine’, ‘correct’, and ‘groupdedup’. The resulting PCR-duplicate-corrected BAM files were aligned to the hg38 reference genome as recommended by PacBio (Echols & Rowell, W. PacificBiosciences/reference_genomes) using ‘pbmm2’ version 1.13.1 (Pacific Biosciences of California, Inc.) with program ‘align’ and options ‘--sort’ and ‘--preset ISOSEQ’. Gene fusions were then counted and summarized into Paired-End BED files using ‘pbfusion’ version 0.5.0 (Pacific Biosciences of California, Inc.) with program ‘discover’ using a serialized GTF (general transfer format) file and the option ‘--min-fusion-quality MEDIUM’.

Prophetic Example. Interrogating HLA perturbations in Acute Myeloid Leukemia. Acute Myeloid Leukemia (AML) is an aggressive blood cancer that often requires personalized treatment strategies based on risk stratification and disease characteristics. Patients with persistent residual disease after initial chemotherapy and those with specific high-risk leukemia-associated gene variants (Samra et al., J. Hematol. Oncol.J Hematol Oncol 13, 70 (2020); Oliansky, et al., J. Am. Soc. Blood Marrow Transplant. 18, 505-522 (2012); Estey, Am. J. Hematol. 93, 1267-1291 (2018); Elgarten & Aplenc, Curr. Opin. Pediatr. 32, 57-66 (2020)) are considered for allogeneic hematopoietic cell transplantation (alloHCT) (Della Starza et al., Front. Oncol. 9, 726 (2019); Heuser et al., Blood 138, 2753-2767 (2021)). However, alloHCT poses significant risks of transplant-related mortality or devastating life-long complications making it unsuitable for every patient. When alloHCT fails to prevent relapse, salvage interventions include donor-lymphocyte infusion (DLI), hypomethylating agents (e.g., azacytidine), immune checkpoint inhibitors, bispecific T-cell engagers (Tian et al., J. Hematol. Oncol. J Hematol Oncol 14, 75 (2021)), chimeric antigen receptors, or a second transplant. Yet, it remains significantly challenging to identify which therapy (often associated with life-threatening secondary effects) to benefit which patient and when. For example, DLI has value in relapse (Schmid et al., J. Clin. Oncol. Off. J. Am. Soc. Clin. Oncol. 25, 4938-4945 (2007)) but is completely ineffective when adaptive loss of an HLA haplotype occurs in the patient's leukemia (Schmid et al., Bone Marrow Transplant. 57, 215-223 (2022)). Drugs such as azacytidine have shown promise when disease levels are low (Platzbecker et al., Leukemia 26, 381-389 (2012)) and are thought to work via increasing expression of HLA. These examples underscore the urgent clinical need to refine disease characterization to guide optimal treatment strategies, particularly as personalized medicine becomes more prominent and the range of available therapeutic interventions grows.

Human Leukocyte Antigens (HLA), the most polymorphic genes in the human genome (Horton et al., Nat. Rev. Genet. 5, 889-899 (2004)), play a central role in immune recognition and Graft-vs.-Leukemia (GVL) effects of alloHCT (Nowak et al., Bone Marrow Transplant. 42 Suppl 2, S71-76 (2008)). A frequent mechanism of immune escape and relapse after alloHCT involves the downregulation of the allele-specific loss of HLA surface expression on leukemia blasts (Toffalori et al., nat. Med. 25, 603-611 (2019): Rovatti et al., Front. Immunol. 11, 147 (2020)). These alterations arise from diverse genetic mechanisms (Toffalori et al., Nat. Med. 25, 603-611 (2019); Christopher et al., N. Engl. J. Med. 379, 2330-2341 (2018): Vago Luca et al., N. Engl. J. Med. 361, 478-488 (2009); Pagliuca et al., Nat. Commun. 14, 3153 (2023)), making it crital to identify the specific cause and affected allele to guide effective treatment options.

To address this critical need, it is essential to identify HLA alleles while simultaneously quantifying their expression. As HLA-mediated immune escape is common after transplant, it is vital to disambiguate the various ways HLA is dysregulated in leukemia to tailor treatment appropriately (FIG. 1B).

Without being bound by theory, it is hypothesized that unbalanced allelic expression of HLA is present at AML diagnosis and contributes to disease relapse. To confirm this hypothesis, scrHLA-typing will be used to probe allelic imbalance retrospectively in AML patients, some of whom went on to relapse and some of whom who did not. In this Example, response to treatment will be defined as patients who experienced 5-year relapse-free survival. Failure will be defined as any patient who relapsed within 5 years. Receipt of alloHCT will not be factored into the analysis.

scrHLA-typing will begin with unfragmented cell-barcoded (CB) cDNA from 5-prime single-cell RNA-seq libraries. The 10× genomics platform will be used for scrHLA-typing, but there are no specific requirements that this platform be used if an alternative produces barcoded full-length transcripts. Briefly, cDNA will be incubated with 5-prime biotinylated hybridization probes HLA-specific and blocking oligos to prevent PCR artifacts. After incubation, the biotinylated probes will be bound to streptavidin magnetic beads, allowing for the enrichment of HLA target sequences. These molecules will then be PCR-amplified and long-read sequenced (FIG. 2E). Software to iteratively align reads, predict genotypes, filter errors, and summarize HLA counts will be used. Cells will be annotated using viewmastR, a machine-learning framework developed to classify cells in the query dataset against a known reference (furlan-lab.github.io/viewmastR/index.html), a well annotated atlas of healthy bone marrow mononuclear cells (Granja et al., Nat. Biotechnol. 37, 1458-1465 (2019)). viewmastR will help identify leukemic cells from putative “healthy” cells. Allelic imbalance will be measured using a standard differential expression measure that fits a regression model to each gene using negative binomial distribution. Patients exhibiting allelic imbalance will then be enumerated and cohorts will be compared using a chi-square test. The single-cell nature of these data will also allow examination of allelic imbalance heterogeneity within patients and correlate this with outcome.

### (x) Closing Paragraphs

Functional variants of sequences disclosed herein (e.g., probes and blocking primers) retain sufficient sequence similarity to hybridize to target sequences of interest under hybridization conditions disclosed within the Experimental Examples section of this disclosure. Further, the disclosure is not limited to the particularly disclosed sequences but instead also encompasses sequences including 40% sequence identity; 45% sequence identity; 50% sequence identity; 55% sequence identity; 60% sequence identity; 65% sequence identity; 70% sequence identity; 75% sequence identity; 80% sequence identity; 81% sequence identity; 82% sequence identity; 83% sequence identity; 84% sequence identity; 85% sequence identity; 86% sequence identity; 87% sequence identity; 88% sequence identity; 89% sequence identity; 90% sequence identity; 91% sequence identity; 92% sequence identity; 93% sequence identity; 94% sequence identity; 95% sequence identity; 96% sequence identity; 97% sequence identity; 98% sequence identity or 99% sequence identity to a sequence referenced herein.

“% sequence identity” refers to a relationship between two or more sequences, as determined by comparing the sequences. In the art, “identity” also means the degree of sequence relatedness between sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine sequence identity are designed to give the best match between the sequences tested. Methods to determine sequence identity and similarity can be found in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.](1994), Meeting Date 1992, 111-20. Editor(s): Suhai, Sandor. Publisher: Plenum, New York, N.Y. Within the context of this disclosure, it will be understood that where sequence analysis software is used for analysis, the results of the analysis are based on the “default values” of the program referenced. “Default values” mean any set of values or parameters which originally load with the software when first initialized.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” As used herein, the transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. As used herein, a material effect would cause a statistically-significant reduction in ability to distinguish HLA variants or expression levels.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that can vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose alimitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that can be employed are within the scope of the invention. Thus, by way of example, but not of limitation, alternative configurations of the present invention can be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention can be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3^{rd} Edition or a dictionary known to those of ordinary skill in the art, such as the Oxford Dictionary of Biochemistry and Molecular Biology (Ed. Anthony Smith, Oxford University Press, Oxford, 2004).

What is claimed is:

1.-80. (canceled)

81. A panel of hybridization probes that hybridize to HLA alleles in HLA regions HLA-A, -B, -C, -E, -F, -G, -H, -J, -K, -L, -T, -V, -W, -Y, -DMA, -DMB, -DOA, -DOB, -DPA1, -DPA2, -DPB1, -DPB2, -DQA1, -DQA2, -DQB1, -DRA, -DRB1, -DRB2, -DRB3, -DRB4, -DRB5, -DRB6, -DRB7, -DRB8, -DRB9, HFE, MICA, MICB, TAP1, and TAP,
wherein the panel of hybridization probes comprises at least five probes selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, and SEQ ID NO: 33
or sequences having at least 99% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, or SEQ ID NO: 33,
wherein R=A/G, Y=C/T, M=A/C, K=G/T, S=C/G, W=A/T, B=C/G/T, D=A/G/T, H=A/C/T, V=A/C/G, and N=A/C/G/T.

82. The panel of claim 81, comprising SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, and SEQ ID NO: 33
or a sequence having at least 99% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, or SEQ ID NO: 33,
wherein R=A/G, Y=C/T, M=A/C, K=G/T, S=C/G, W=A/T, B=C/G/T, D=A/G/T, H=A/C/T, V=A/C/G, and N=A/C/G/T.

83. The panel of claim 81, comprising SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, and SEQ ID NO: 33
wherein R=A/G, Y=C/T, M=A/C, K=G/T, S=C/G, W=A/T, B=C/G/T, D=A/G/T, H=A/C/T, V=A/C/G, and N=A/C/G/T

84. The panel of claim 81, further comprising SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, or SEQ ID NO: 69
or sequences having at least 99% sequence identity to SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, or SEQ ID NO: 69
wherein R=A/G, Y=C/T, M=A/C, K=G/T, S=C/G, W=A/T, B=C/G/T, D=A/G/T, H=A/C/T, V=A/C/G, and N=A/C/G/T.

85. The panel of claim 81, further comprising SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID Page 3 of 6 NO: 62, SEQ ID NO: 63, SEQ ID NO: 64, SEQ ID NO: 65, SEQ ID NO: 66, SEQ ID NO: 67, SEQ ID NO: 68, and SEQ ID NO: 69
or sequences having at least 99% sequence identity to SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, and SEQ ID NO: 69
wherein R=A/G, Y=C/T, M=A/C, K=G/T, S=C/G, W=A/T, B=C/G/T, D=A/G/T, H=A/C/T, V=A/C/G, and N=A/C/G/T.

86. The panel of claim 81, further comprising SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, and SEQ ID NO: 69
wherein R=A/G, Y=C/T, M=A/C, K=G/T, S=C/G, W=A/T, B=C/G/T, D=A/G/T, H=A/C/T, V=A/C/G, and N=A/C/G/T.

87. The panel of claim 81, further comprising a blocking oligo comprising SEQ ID NO: 34, SEQ ID NO: 35, SEQ ID NO: 70, or SEQ ID NO: 36 or a sequence having at least 99% sequence identity to SEQ ID NO: 34, SEQ ID NO: 35, SEQ ID NO: 70, or SEQ ID NO: 36.

88. The panel of claim 81, further comprising a blocking oligo comprising SEQ ID NO: 34, SEQ ID NO: 35, SEQ ID NO: 70, or SEQ ID NO: 36.

89. The panel of claim 81, wherein the probes are linked to a detectable label.

90. The panel of claim 81, wherein the probes are biotinylated.

91. The panel of claim 81, wherein the probes are linked to a detectable label at the 5′ end of each probe.

92. A method comprising
exposing a cDNA sample to the panel of claim 81, the exposing resulting in captured cDNA;
polymerase chain reaction (PCR) amplifying the captured DNA, the amplifying resulting in amplified DNA.

93. The method of claim 92, wherein the captured cDNA includes a barcode and a unique molecular identifier.

94. The method of claim 92, wherein the PCR amplifying is in the presence of blocking oligos.

95. The method of claim 94, wherein the blocking oligos comprise SEQ ID NO: 34, SEQ ID NO: 35, SEQ ID NO: 70, or SEQ ID NO: 36 or a sequence having at least 99% sequence identity to SEQ ID NO: 34, SEQ ID NO: 35, SEQ ID NO: 70, or SEQ ID NO: 36.

96. The method of claim 92, further comprising sequencing the amplified DNA.

97. The method of claim 96, further identifying an HLA allele expressed by a cell based on the sequencing.

98. The method of claim 97, wherein the cell is a cancer cell.

99. The method of claim 98, wherein the cancer cell is a leukemia cell.

100. The method of claim 99, wherein the leukemia cell is an acute myeloid leukemia (AML) cell, acute lymphoblastic leukemia (ALL) cell, chronic myelogenous leukemia (CML) cell, chronic myelomonocytic leukemia (CML) cell, mast cell leukemia cell, myelodysplastic syndrome (MDS) cell, B-cell acute lymphoblastic leukemia (B-ALL) cell, T-cell acute lymphoblastic leukemia (T-ALL) cell, or megakaryocytic leukemia cell.


SEQUENCE LISTING (82 sequences)

SEQ ID NO: 1 - DNA, 97 bp, synthetic construct
  source 1..97: mol_type = other DNA
accaggcctgcaggggatggaaccttccagaagtgggcgkctgtggtggtrccttctggasaggagcagagatacacctgccatgtgcagcatgagg

SEQ ID NO: 2 - DNA, 99 bp, synthetic construct
  source 1..99: mol_type = other DNA
gtytscccaagcccctcaccctgagatgggagcygtcttcccagcccaccatccccatcgtgggcatcattgctggcctggttctcyttggagctgtgw

SEQ ID NO: 3 - DNA, 92 bp, synthetic construct
  source 1..92: mol_type = other DNA
ggcagctgtggtggtgccttctggagaagagcagagatacacatgccatgtacagcatgaggggctgccraagcccctcaccctgagatggg

SEQ ID NO: 4 - DNA, 93 bp, synthetic construct
  source 1..93: mol_type = other DNA
acytggggtccgacggwcgcctcmtccgcgggtatgaacagtatgcctacgatggcaaggattacctcgccctgaacgaggacctgcgctcct

SEQ ID NO: 5 - DNA, 98 bp, synthetic construct
  source 1..98: mol_type = other DNA
gcgagtggatgatccagcaaatagggccaaaacttgatgggaaaatcccggtgtccagagggtttcctatcgctgaagtgttcacgctgaagcccctg

SEQ ID NO: 6 - DNA, 98 bp, synthetic construct
  source 1..98: mol_type = other DNA
gagtttggcaagcccaacactttggtctgttttgtcagtaatctcttcccacccatgctgacagtgaactggcagcatcattccrtccctgtggaagg

SEQ ID NO: 7 - DNA, 89 bp, synthetic construct
  source 1..89: mol_type = other DNA
cccctcttacggggacacttacacctgtgtggtagagcacaytggggctcctgagcccatccttcgggactggacacctgggctgtccc

SEQ ID NO: 8 - DNA, 96 bp, synthetic construct
  source 1..96: mol_type = other DNA
ccatgcagaccctgaaggtttctgtgtctgcagtgactctgggcctgggcctcatcatcttctctcttggtgtgatcagctggcggagagctggcc

SEQ ID NO: 9 - DNA, 98 bp, synthetic construct
  source 1..98: mol_type = other DNA
gggccagcccaacatcctcatctgcatcgtggacaacatcttcccccctgtgatcaatatcacctggctrcgcaacggccaaactgtcactgagggag

SEQ ID NO: 10 - DNA, 97 bp, synthetic construct
  source 1..97: mol_type = other DNA
tggcccagaccagcttctattcccagcctgaccatttgttccgcaagttccactacctgcccttcgtgccctcagccgaggacgtctatgactgcca

SEQ ID NO: 11 - DNA, 94 bp, synthetic construct
  source 1..94: mol_type = other DNA
gggatgtttgtggcattgaccaagctggggcagccagatgctgagcagtggaacagccggctggatctcttggaraggagcagacaggccgtgg

SEQ ID NO: 12 - DNA, 94 bp, synthetic construct
  source 1..94: mol_type = other DNA
atggggtctgtagacacaactacaggctgggcgcacccttcactgtggggagaaaagtgcaaccagaggtgacagtgtacccagagaggacccc

SEQ ID NO: 13 - DNA, 91 bp, synthetic construct
  source 1..91: mol_type = other DNA
ccagtgctcaacgtcacrtggctgtgcaayggggagcyggtcactgagggtgtcgctgagagcctcttcctgcccagaacagattacagct

SEQ ID NO: 14 - DNA, 91 bp, synthetic construct
  source 1..91: mol_type = other DNA
tccacaagttccattacctgacctttgtgccctcagcagaggacktctatgactgcagggtggagcactggggcttggaccagccgctcct

SEQ ID NO: 15 - DNA, 95 bp, synthetic construct
  source 1..95: mol_type = other DNA
aggtggagcactggggcctggaysagcctcttctgaaacactgggagcctgagattccagcmcctatgtcagagctcacagagactktggtctgy

SEQ ID NO: 16 - DNA, 96 bp, synthetic construct
  source 1..96: mol_type = other DNA
rccctgggrttgtctgtgggcctcrtgggcattgtggtgggcactgtcttcatcatccraggcctgcgttcagttggtgcttccagacaccarggg

SEQ ID NO: 17 - DNA, 91 bp, synthetic construct
  source 1..91: mol_type = other DNA
gggcccytgcagcaccacaacctgcttgtctgccacgtgacrgatttctacccaggcagcattcaagtccgatggttcctgaatggacagg

SEQ ID NO: 18 - DNA, 91 bp, synthetic construct
  source 1..91: mol_type = other DNA
aggaaacagctggggtcgtgtccaccaacctgatccgtaatggagactggaccttccagatcctggtgatgctggaaatgaccccccagca

SEQ ID NO: 19 - DNA, 96 bp, synthetic construct
  source 1..96: mol_type = other DNA
tccccatccaggacagaggccctcaaccaccacaacctgctgrtctgctcrgtgacagatttctatccagsccagatcaaagtccggtggtttcgg

SEQ ID NO: 20 - DNA, 98 bp, synthetic construct
  source 1..98: mol_type = other DNA
aatgrycaggaggagacarcyggcgttgtgtccaccccccttattaggaayggtgactggaccttccagatcctggtgatgctggaaatgactcccca

SEQ ID NO: 21 - DNA, 93 bp, synthetic construct
  source 1..93: mol_type = other DNA
gggaagaccaccttttccgcaagttccactatctccccttcctgccctcaactgaggacgtttacgactgcagggtggagcactggggcttgg

SEQ ID NO: 22 - DNA, 94 bp, synthetic construct
  source 1..94: mol_type = other DNA
atgagcctcttctcaagcactgggagtttgatgctccaagccctctcccagagactacagagaaygtggtgtgtgccctgggcctgactgtggg

SEQ ID NO: 23 - DNA, 94 bp, synthetic construct
  source 1..94: mol_type = other DNA
tkcaaagacccagcccctgcagcaccacaacctcctggtctgytctgtgartggtttctatccaggcagcattgaagtcaggtggttcckgaay

SEQ ID NO: 24 - DNA, 96 bp, synthetic construct
  source 1..96: mol_type = other DNA
rgccaggaagagaagrctggggtggtgtccacrggcctgatccasaatggagactggaccttccagaccctggtgatgctggaaacagttcctcgg

SEQ ID NO: 25 - DNA, 93 bp, synthetic construct
  source 1..93: mol_type = other DNA
gcggagctggagcggagaagagtccaggaatrcaaacagccagaagaacctcttgggctgcttgcggggtctgttggacacctactgcagaca

SEQ ID NO: 26 - DNA, 95 bp, synthetic construct
  source 1..95: mol_type = other DNA
gccctgacacactggattggagagcagcagaacccagggcctggcccaccaagctggagtgggaaaggcacaagattcgggccaggcagaacagg

SEQ ID NO: 27 - DNA, 97 bp, synthetic construct
  source 1..97: mol_type = other DNA
gcctacctggagagggactgccctgcacagctgcagcagttgctggagctggggagaggtgttttggaccaacaagtgcctcctttggtgaaggtga

SEQ ID NO: 28 - DNA, 73 bp, synthetic construct
  source 1..73: mol_type = other DNA
agtcagggtttctyrctgaggtacatctggatggtcagcccttcctgcgctrtgacaggcagaaaygcagggc

SEQ ID NO: 29 - DNA, 77 bp, synthetic construct
  source 1..77: mol_type = other DNA
aaagccccagggacagtgggcagaagatgtcctgggaaataagacatgggacagagagaccagrgacttgacaggga

SEQ ID NO: 30 - DNA, 94 bp, synthetic construct
  source 1..94: mol_type = other DNA
tggggatcagtgtccctcaccatggtcaccctgrtcaccctgcctctgcttttccttctgcccaagaaggtgggaaaatggtaccagttgctgg

SEQ ID NO: 31 - DNA, 96 bp, synthetic construct
  source 1..96: mol_type = other DNA
aagtgcaggtgcgggaatctctggcaaagtccagccaggtggccattgaggyyctgtcggccatgcctacagttcgaagctttgccaacgaggagg

SEQ ID NO: 32 - DNA, 91 bp, synthetic construct
  source 1..91: mol_type = other DNA
gccgttggagggctgcagaccgttcgcagttttggggccgaggagcatgaagtctgtcgctataaagaggcccttgaacaatgtcggcagc

SEQ ID NO: 33 - DNA, 91 bp, synthetic construct
  source 1..91: mol_type = other DNA
tgtattggcggagagacctggaacgcgccttgtacctgctcrtaaggagggtgctgcacttgggkgtgcagatgctgatgctgagctgtgg

SEQ ID NO: 34 - DNA, 61 bp, synthetic construct
  source 1..61: mol_type = other DNA
  modified_base 23..48: mod_base = i
ctacacgacgctcttccgatctnnnnnnnnnnnnnnnnnnnnnnnnnntttcttatatggg

SEQ ID NO: 35 - DNA, 45 bp, synthetic construct
  source 1..45: mol_type = other DNA
aaaaaaaaaaaaaaaaaaaagtactctgcgttgataccactgctt

SEQ ID NO: 36 - DNA, 70 bp, synthetic construct
  source 1..70: mol_type = other DNA
  modified_base 21..48: mod_base = i
aaaaaaaaaaaaaaaaaaaannnnnnnnnnnnnnnnnnnnnnnnnnnnagatcggaagagcgtcgtgtag

SEQ ID NO: 37 - DNA, 101 bp, synthetic construct
  source 1..101: mol_type = other DNA
accaggcctgcaggggatggaaccttccagaagtgggcgkctgtggtggtrccttctggasaggagcagagatacacctgccatgtgcagcatgaggaaaa

SEQ ID NO: 38 - DNA, 103 bp, synthetic construct
  source 1..103: mol_type = other DNA
gtytscccaagcccctcaccctgagatgggagcygtcttcccagcccaccatccccatcgtgggcatcattgctggcctggttctcyttggagctgtgwaaaa

SEQ ID NO: 39 - DNA, 96 bp, synthetic construct
  source 1..96: mol_type = other DNA
ggcagctgtggtggtgccttctggagaagagcagagatacacatgccatgtacagcatgaggggctgccraagcccctcaccctgagatgggaaaa

SEQ ID NO: 40 - DNA, 97 bp, synthetic construct
  source 1..97: mol_type = other DNA
acytggggtccgacggwcgcctcmtccgcgggtatgaacagtatgcctacgatggcaaggattacctcgccctgaacgaggacctgcgctcctaaaa

SEQ ID NO: 41 - DNA, 102 bp, synthetic construct
  source 1..102: mol_type = other DNA
gcgagtggatgatccagcaaatagggccaaaacttgatgggaaaatcccggtgtccagagggtttcctatcgctgaagtgttcacgctgaagcccctgaaaa

SEQ ID NO: 42 - DNA, 102 bp, synthetic construct
  source 1..102: mol_type = other DNA
gagtttggcaagcccaacactttggtctgttttgtcagtaatctcttcccacccatgctgacagtgaactggcagcatcattccrtccctgtggaaggaaaa

SEQ ID NO: 43 - DNA, 93 bp, synthetic construct
  source 1..93: mol_type = other DNA
cccctcttacggggacacttacacctgtgtggtagagcacaytggggctcctgagcccatccttcgggactggacacctgggctgtcccaaaa

SEQ ID NO: 44 - DNA, 100 bp, synthetic construct
  source 1..100: mol_type = other DNA
ccatgcagaccctgaaggtttctgtgtctgcagtgactctgggcctgggcctcatcatcttctctcttggtgtgatcagctggcggagagctggccaaaa

SEQ ID NO: 45 - DNA, 102 bp, synthetic construct
  source 1..102: mol_type = other DNA
gggccagcccaacatcctcatctgcatcgtggacaacatcttcccccctgtgatcaatatcacctggctrcgcaacggccaaactgtcactgagggagaaaa

SEQ ID NO: 46 - DNA, 101 bp, synthetic construct
  source 1..101: mol_type = other DNA
tggcccagaccagcttctattcccagcctgaccatttgttccgcaagttccactacctgcccttcgtgccctcagccgaggacgtctatgactgccaaaaa

SEQ ID NO: 47 - DNA, 98 bp, synthetic construct
  source 1..98: mol_type = other DNA
gggatgtttgtggcattgaccaagctggggcagccagatgctgagcagtggaacagccggctggatctcttggaraggagcagacaggccgtggaaaa

SEQ ID NO: 48 - DNA, 98 bp, synthetic construct
  source 1..98: mol_type = other DNA
atggggtctgtagacacaactacaggctgggcgcacccttcactgtggggagaaaagtgcaaccagaggtgacagtgtacccagagaggaccccaaaa

SEQ ID NO: 49 - DNA, 95 bp, synthetic construct
  source 1..95: mol_type = other DNA
ccagtgctcaacgtcacrtggctgtgcaayggggagcyggtcactgagggtgtcgctgagagcctcttcctgcccagaacagattacagctaaaa

SEQ ID NO: 50 - DNA, 95 bp, synthetic construct
  source 1..95: mol_type = other DNA
tccacaagttccattacctgacctttgtgccctcagcagaggacktctatgactgcagggtggagcactggggcttggaccagccgctcctaaaa

SEQ ID NO: 51 - DNA, 99 bp, synthetic construct
  source 1..99: mol_type = other DNA
aggtggagcactggggcctggaysagcctcttctgaaacactgggagcctgagattccagcmcctatgtcagagctcacagagactktggtctgyaaaa

SEQ ID NO: 52 - DNA, 100 bp, synthetic construct
  source 1..100: mol_type = other DNA
rccctgggrttgtctgtgggcctcrtgggcattgtggtgggcactgtcttcatcatccraggcctgcgttcagttggtgcttccagacaccargggaaaa

SEQ ID NO: 53 - DNA, 95 bp, synthetic construct
  source 1..95: mol_type = other DNA
gggcccytgcagcaccacaacctgcttgtctgccacgtgacrgatttctacccaggcagcattcaagtccgatggttcctgaatggacaggaaaa

SEQ ID NO: 54 - DNA, 95 bp, synthetic construct
  source 1..95: mol_type = other DNA
aggaaacagctggggtcgtgtccaccaacctgatccgtaatggagactggaccttccagatcctggtgatgctggaaatgaccccccagcaaaaa

SEQ ID NO: 55 - DNA, 100 bp, synthetic construct
  source 1..100: mol_type = other DNA
tccccatccaggacagaggccctcaaccaccacaacctgctgrtctgctcrgtgacagatttctatccagsccagatcaaagtccggtggtttcggaaaa

SEQ ID NO: 56 - DNA, 102 bp, synthetic construct
  source 1..102: mol_type = other DNA
aatgrycaggaggagacarcyggcgttgtgtccaccccccttattaggaayggtgactggaccttccagatcctggtgatgctggaaatgactccccaaaaa

SEQ ID NO: 57 - DNA, 97 bp, synthetic construct
  source 1..97: mol_type = other DNA
gggaagaccaccttttccgcaagttccactatctccccttcctgccctcaactgaggacgtttacgactgcagggtggagcactggggcttggaaaa

SEQ ID NO: 58 - DNA, 98 bp, synthetic construct
  source 1..98: mol_type = other DNA
atgagcctcttctcaagcactgggagtttgatgctccaagccctctcccagagactacagagaaygtggtgtgtgccctgggcctgactgtgggaaaa

SEQ ID NO: 59 - DNA, 98 bp, synthetic construct
  source 1..98: mol_type = other DNA
tkcaaagacccagcccctgcagcaccacaacctcctggtctgytctgtgartggtttctatccaggcagcattgaagtcaggtggttcckgaayaaaa

SEQ ID NO: 60 - DNA, 100 bp, synthetic construct
  source 1..100: mol_type = other DNA
rgccaggaagagaagrctggggtggtgtccacrggcctgatccasaatggagactggaccttccagaccctggtgatgctggaaacagttcctcggaaaa

SEQ ID NO: 61 - DNA, 97 bp, synthetic construct
  source 1..97: mol_type = other DNA
gcggagctggagcggagaagagtccaggaatrcaaacagccagaagaacctcttgggctgcttgcggggtctgttggacacctactgcagacaaaaa

SEQ ID NO: 62 - DNA, 99 bp, synthetic construct
  source 1..99: mol_type = other DNA
gccctgacacactggattggagagcagcagaacccagggcctggcccaccaagctggagtgggaaaggcacaagattcgggccaggcagaacaggaaaa

SEQ ID NO: 63 - DNA, 101 bp, synthetic construct
  source 1..101: mol_type = other DNA
gcctacctggagagggactgccctgcacagctgcagcagttgctggagctggggagaggtgttttggaccaacaagtgcctcctttggtgaaggtgaaaaa

SEQ ID NO: 64 - DNA, 77 bp, synthetic construct
  source 1..77: mol_type = other DNA
agtcagggtttctyrctgaggtacatctggatggtcagcccttcctgcgctrtgacaggcagaaaygcagggcaaaa

SEQ ID NO: 65 - DNA, 81 bp, synthetic construct
  source 1..81: mol_type = other DNA
aaagccccagggacagtgggcagaagatgtcctgggaaataagacatgggacagagagaccagrgacttgacagggaaaaa

SEQ ID NO: 66 - DNA, 98 bp, synthetic construct
  source 1..98: mol_type = other DNA
tggggatcagtgtccctcaccatggtcaccctgrtcaccctgcctctgcttttccttctgcccaagaaggtgggaaaatggtaccagttgctggaaaa

SEQ ID NO: 67 - DNA, 100 bp, synthetic construct
  source 1..100: mol_type = other DNA
aagtgcaggtgcgggaatctctggcaaagtccagccaggtggccattgaggyyctgtcggccatgcctacagttcgaagctttgccaacgaggaggaaaa

SEQ ID NO: 68 - DNA, 95 bp, synthetic construct
  source 1..95: mol_type = other DNA
gccgttggagggctgcagaccgttcgcagttttggggccgaggagcatgaagtctgtcgctataaagaggcccttgaacaatgtcggcagcaaaa

SEQ ID NO: 69 - DNA, 95 bp, synthetic construct
  source 1..95: mol_type = other DNA
tgtattggcggagagacctggaacgcgccttgtacctgctcrtaaggagggtgctgcacttgggkgtgcagatgctgatgctgagctgtggaaaa

SEQ ID NO: 70 - DNA, 30 bp, synthetic construct
  source 1..30: mol_type = other DNA
aagcagtggtatcaacgcagagtacatggg

SEQ ID NO: 71 - DNA, 22 bp, synthetic construct
  source 1..22: mol_type = other DNA
ctacacgacgctcttccgatct

SEQ ID NO: 72 - DNA, 22 bp, synthetic construct
  source 1..22: mol_type = other DNA
aagcagtggtatcaacgcagag

SEQ ID NO: 73 - DNA, 25 bp, synthetic construct
  source 1..25: mol_type = other DNA
  modified_base 1: mod_base = OTHER; note = uracil
  modified_base 3: mod_base = OTHER; note = uracil
tataagcagtggtatcaacgcagag

SEQ ID NO: 74 - DNA, 25 bp, synthetic construct
  source 1..25: mol_type = other DNA
  modified_base 1: mod_base = OTHER; note = uracil
  modified_base 3: mod_base = OTHER; note = uracil
tatctacacgacgctcttccgatct

SEQ ID NO: 75 - DNA, 36 bp, synthetic construct
  source 1..36: mol_type = other DNA
atttaagctcaatgacatctccattcttctctttta

SEQ ID NO: 76 - DNA, 25 bp, synthetic construct
  source 1..25: mol_type = other DNA
ttatctctacttttccaggcccagg

SEQ ID NO: 77 - DNA, 27 bp, synthetic construct
  source 1..27: mol_type = other DNA
tcaaagctgttccgtcccagtagatta

SEQ ID NO: 78 - DNA, 94 bp, synthetic construct
  source 1..94: mol_type = other DNA
gctccccgcccaagtatccctgtaaaacaaaaaccaaaagaaaagtctgaacaacccagtcctgccagctccagctccagctccagctccaaaa

SEQ ID NO: 79 - DNA, 94 bp, synthetic construct
  source 1..94: mol_type = other DNA
gctccccgcccaagtatccctgtaaaacaaaaaccaaaagaaaagacttatacaagcactagcaacaactctatatctggatcattgaagaaaa

SEQ ID NO: 80 - DNA, 94 bp, synthetic construct
  source 1..94: mol_type = other DNA
gtggtttcatggacatctctctgggaaagaagcagagaaattattaactgaaaaaggaaaacatggtagttttcttgtacgagagagccaaaaa

SEQ ID NO: 81 - DNA, 94 bp, synthetic construct
  source 1..94: mol_type = other DNA
ctatgatttaggcatagagaatcgtgatgccaccaacgaccaagtcaccaaggatgctgcagaagctataaagaagcataatgttggcgtaaaa

SEQ ID NO: 82 - DNA, 94 bp, synthetic construct
  source 1..94: mol_type = other DNA
tactcccctgccctcaacaagatgttttgccaactggccaagacctgccctgtgcagctgtgggttgattccacacccccgcccggcaccaaaa